(19) United States
(12) Patent Application Publication
(10) Pub. No.: US 2015/0126364 A1
(43) Pub. Date: May 7, 2015

(54) 1,3-DIARYL-SUBSTITUTED HETEROCYCLIC PESTICIDES

(71) Applicant: E I DU PONT DE NEMOURS AND COMPANY, Wilmington, DE (US)

(72) Inventors: Thomas Francis Pahutski, JR., Elkton, MD (US); Matthew James Campbell, Rising Sun, MD (US); Dominic Ming-Tak Chan, Wilmington, DE (US); Jeffrey Keith Long, Wilmington, DE (US); Thomas Martin Stevenson, Newark, DE (US)

(73) Assignee: E I Du Pont de Nemours and Company, Wilimington, DE (US)

(21) Appl. No.: 14/400,872

(22) PCT Filed: May 13, 2013
(86) PCT No.: PCT/US2013/040748

§ 371 Date: Nov. 13, 2014

Related U.S. Application Data
(60) Provisional application No. 61/647,659, filed on May 16, 2012.

(51) Int. Cl. C07D 413/14; A01N 43/56; C07D 401/14; A01N 43/82; A01N 43/60; A01N 43/84; C07D 403/06; A01N 43/50; C07D 401/06; C07D 231/12

(52) U.S. Cl. CPC C07D 413/14; C07D 401/06; A01N 43/56; C07D 401/14; C07D 231/12; A01N 43/60; A01N 43/84; C07D 403/06; A01N 43/50; A01N 43/82
USPC 504/100; 546/211; 514/326; 544/322; 514/256; 544/371; 514/254.05; 544/140; 514/236.5; 548/364.1; 514/210.2; 546/210; 546/194; 514/318; 546/209

(57) ABSTRACT

Disclosed are compounds of Formula 1, N-oxides, and salts thereof,

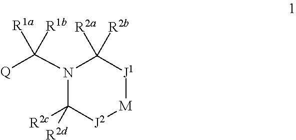

wherein
Q is

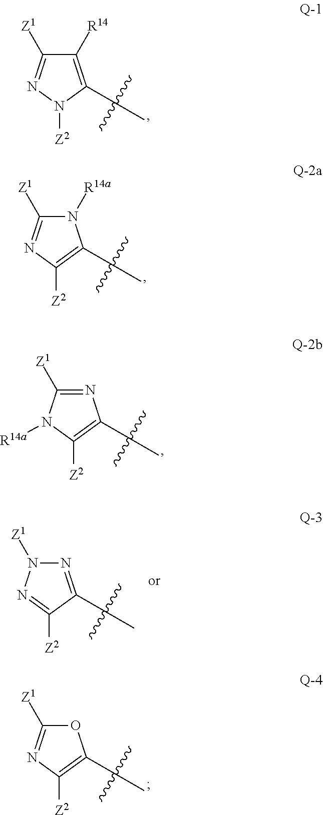

and Z¹, Z², J¹, J², M, R^{1a}, R^{1b}, R^{2a}, R^{2b}, R^{2c}, R^{2d}, R¹⁴ and R^{14a} are as defined in the disclosure. Also disclosed are compositions containing the compounds of Formula 1 and methods for controlling an invertebrate pest comprising contacting the invertebrate pest or its environment with a biologically effective amount of a compound or a composition of the invention.

### FIELD OF THE INVENTION

This invention relates to certain 1,3-diaryl-substituted heterocyclic compounds, their N-oxides, salts and their compositions suitable for agronomic and nonagronomic uses, and methods of their use for controlling invertebrate pests such as arthropods in both agronomic and nonagronomic environments.

### BACKGROUND OF THE INVENTION

The control of invertebrate pests is extremely important in achieving high crop efficiency. Damage by invertebrate pests to growing and stored agronomic crops can cause significant reduction in productivity and thereby result in increased costs to the consumer. The control of invertebrate pests in forestry, greenhouse crops, ornamentals, nursery crops, stored food and fiber products, livestock, household, turf, wood products, and public health is also important. Many products are commercially available for these purposes, but the need continues for new compounds that are more effective, less costly, less toxic, environmentally safer or have different sites of action.

U.S. Pat. No. 7,566,709 B2 discloses pyrazole compounds of Formula i as 5-HT receptor antagonists for the treatment of psychoses and neurological disorders

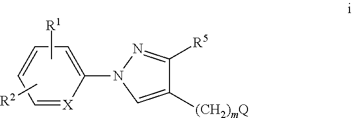

wherein, inter alia, R¹ and R² are independently H, A or halogen, X is N or CH, R⁵ is alkyl or an aromatic ring, A is alkyl, and Q is NR³R⁴or a heteroatom-containing radical.

### SUMMARY OF THE INVENTION

This invention is directed to compounds of Formula 1 (including all stereoisomers), N-oxides, and salts thereof:

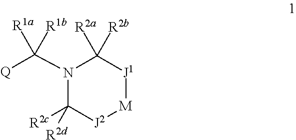

wherein

Q is

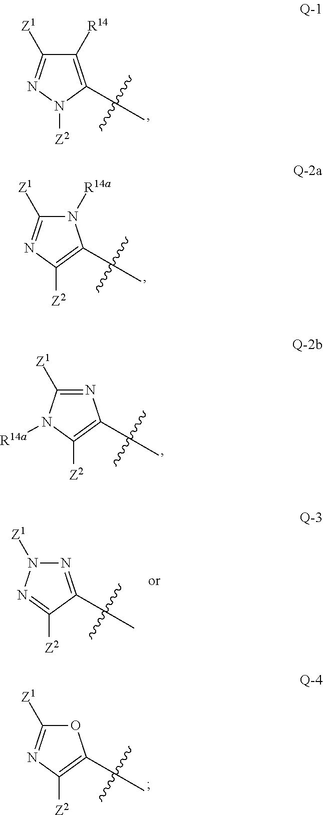

R^{1a} is H, cyano, C₁-C₆ alkyl, C₃-C₆ cycloalkyl, C₁-C₆ haloalkyl, C(O)OH, C(O)R^{5a}, C(O)OR^{6a} or C(O)NR^{7a}R^{8a};
R^{1b} is H or C₁-C₆ alkyl;
R^{2a} and R^{2c} are each independently H, halogen, cyano, C(X)R⁵, C(O)OR⁶, C(X)NR⁷R⁸, OR¹² or S(O)ₙR¹¹; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a};
R^{2b} and R^{2d} are each independently H, halogen, cyano, nitro, C(X)R⁵, C(O)OR⁶, C(X)NR⁷R⁸, NR⁹R¹⁰, OR¹², S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R⁴;
J¹ is a direct bond, —C(R^{3a}R^{3b})— or —C(R^{3a}R^{3b})C(R^{3a}R^{3b})—;
J² is a direct bond or —C(R^{3c}R^{3d})—;
M is —C(R^{3e})(A)-, —N(A¹)-, —O— or —S(O)ₙ—;
A is halogen, cyano, nitro, C(X)R⁵, C(O)OR⁶, C(X)NR⁷R⁸, C(X)NR^{7b}R⁸, NR⁹R¹⁰, S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, C₁-C₄ alkyl, C₁-C₄ haloalkyl, C(O)R^{5a}, C(O)OR^{6a}, C(O)NR^{7a}R^{8a}, NR^{9a}R^{10a}, OR^{12a} and S(O)ₙR^{11a}; or phenyl, a 5- or 6-membered heteroaromatic ring or a 7- to 11-membered heteroaromatic ring system, each ring or ring system unsubstituted or substituted with 1 to 3 R⁴;
A¹ is cyano, nitro, C(X)R⁵, C(O)OR⁶, C(X)NR⁷R⁸, C(X)NR^{7b}R⁸, NR⁹R¹⁰, S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, C₁-C₄ alkyl, C₁-C₄ haloalkyl, C(O)R^{5a}, C(O)OR^{6a}, C(O)NR^{7a}R^{8a}, NR^{9a}R^{10a}, OR^{12a} and S(O)ₙR^{11a}; or phenyl, a 5- or 6-membered heteroaromatic ring or a 7- to 11-membered heteroaromatic ring system, each ring or ring system unsubstituted or substituted with 1 to 3 R⁴; or benzyl unsubstituted or substituted with 1 to 3 R⁴;
each R^{3a} and R^{3c} are each independently H, halogen, cyano, C(X)R⁵, C(O)OR⁶, C(X)NR⁷R⁸, OR¹² or S(O)ₙR¹¹; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a};
each R^{3b} and R^{3d} are each independently H, halogen, cyano, nitro, C(X)R⁵, C(O)OR⁶, C(X)NR⁷R⁸, NR⁹R¹⁰, OR¹², S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R⁴;
R^{3e} is H, halogen, cyano, nitro, C(X)R⁵, C(O)OR⁶, C(X)NR⁷R⁸, NR⁹R¹⁰, OR¹², NR⁹R¹⁰, S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R⁴; or
R^{3e} and A can be taken together with the carbon atom to which they are attached to form a 3- to 7-membered ring containing ring members selected from carbon atoms and up to 2 heteroatoms independently selected from one oxygen atom, one sulfur atom, and up to 2 nitrogen atoms, wherein up to 2 carbon atom ring members are independently selected from C(═O) and C(═S) and the sulfur atom ring member is selected from S, S(O) or S(O)₂, said ring being unsubstituted or substituted with up to 4 substituents independently selected from the group consisting of halogen, cyano and C₁-C₄ alkyl; or
when any two substituents independently selected from the group consisting of R^{2a}, R^{2b}, R^{2c}, R^{2d}, R^{3a}, R^{3b}, R^{3c}, R^{3d} and R^{3e} are C₁-C₄ alkyl, then said two substituents can be taken together to form a ring;
Z¹ is phenyl substituted with 1 to 4 R^{4a}; or Z¹ is a 5- or 6-membered heteroaromatic ring or an 8- to 10-membered heteroaromatic bicyclic ring system, each ring or ring system unsubstituted or substituted with 1 to 3 R^{4a};
Z² is phenyl, unsubstituted or substituted with 1 to 4 R^{4b}; or Z² is a 5- or 6-membered heteroaromatic ring, each ring unsubstituted or substituted with 1 to 3 R^{4b};
each R⁴, R^{4a} and R^{4b} is independently halogen, cyano, nitro, C(X)R⁵, C(O)OR⁶, C(X)NR⁷R⁸, NR⁹R¹⁰, OR¹², S(O)ₙR¹¹ or SO₂NR⁹R¹⁰; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R⁵ is independently H; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{5a} is independently C₁-C₄ alkyl or C₁-C₄ haloalkyl;
each R⁶ is independently C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{6a} is independently C₁-C₄ alkyl or C₁-C₄ haloalkyl;
each R⁷ and R⁸ is independently H; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{7a} and R^{8a} is independently H, C₁-C₄ alkyl or C₁-C₄ haloalkyl;
each R^{7b} is independently N(R^{7a})₂, OH or OR^{12a};
each R⁹ and R¹⁰ is independently H, C(X)R⁵, C(O)OR⁶ or C(X)NR⁷R⁸; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{9a} and R^{10a} is independently H, C(X)R^{5a}, C(O)OR^{6a}, C(X)NR^{7a}R^{8a}, C₁-C₄ alkyl or C₁-C₄ haloalkyl;
each R¹¹ is independently C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{11a} is independently C₁-C₄ alkyl or C₁-C₄ haloalkyl;
each R¹² is independently H; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{12a} is independently H, C₁-C₄ alkyl or C₁-C₄ haloalkyl;
each R¹³ is independently halogen, cyano, nitro, C(X)R^{5a}, C(O)OR^{6a}, C(X)NR^{7a}R^{8a}, NR^{9a}R^{10a}, OR^{12a}, S(O)ₙR^{11a} or SO₂NR^{9a}R^{10a}; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a};
R¹⁴ is H, halogen, cyano, nitro, C(X)R⁵, C(O)OR⁶, C(X)NR⁷R⁸, NR⁹R¹⁰, OR¹², S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a};
R^{14a} is H; or C₁-C₆ alkyl, C₂-C₆ alkenyl, C₂-C₆ alkynyl or benzyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; and when R^{14a} is C₁-C₆ alkyl or C₂-C₆ alkenyl, then R^{14a} can be bonded to Z¹ to form a ring;
each X is independently O or S; and
each n is independently 0, 1 or 2.

This invention also provides a composition comprising a compound of Formula 1, an N-oxide, or a salt thereof, and at least one additional component selected from the group consisting of surfactants, solid diluents and liquid diluents. In one embodiment, this invention also provides a composition for controlling an invertebrate pest comprising a compound of Formula 1, an N-oxide, or a salt thereof, and at least one additional component selected from the group consisting of surfactants, solid diluents and liquid diluents, said composition further comprising at least one additional biologically active compound or agent.

This invention also provides a method for controlling an invertebrate pest comprising contacting the invertebrate pest or its environment with a biologically effective amount of a compound of Formula 1, an N-oxide, or a salt thereof (e.g., as a composition described herein). This invention also relates to such method wherein the invertebrate pest or its environment is contacted with a composition comprising a biologically effective amount of a compound of Formula 1, an N-oxide, or a salt thereof, and at least one additional component selected from the group consisting of surfactants, solid diluents and liquid diluents, said composition optionally further comprising a biologically effective amount of at least one additional biologically active compound or agent.

This invention also provides a method for controlling an invertebrate pest comprising contacting the invertebrate pest or its environment with a biologically effective amount of any of the aforesaid compositions wherein the environment is a plant.

This invention also provides a method for controlling an invertebrate pest comprising contacting the invertebrate pest or its environment with a biologically effective amount of any of the aforesaid compositions wherein the environment is an animal.

This invention also provides a method for controlling an invertebrate pest comprising contacting the invertebrate pest or its environment with a biologically effective amount of any of the aforesaid compositions wherein the environment is a seed.

This invention also provides a method for protecting a seed from an invertebrate pest comprising contacting the seed with a biologically effective amount of a compound of Formula 1, an N-oxide, or a salt thereof (e.g., as a composition described herein). This invention also relates to the treated seed.

### DETAILS OF THE INVENTION

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods and nematodes of economic importance as pests. The term “arthropod” includes insects, mites, spiders, scorpions, centipedes, millipedes, pill bugs and symphylans. The term “gastropod” includes snails, slugs and other Stylommatophora. The term “nematode” refers to a living organism of the Phylum Nematoda. The term “helminths” includes roundworms, heartworms, phytophagous nematodes (Nematoda), flukes (Tematoda), Acanthocephala and tapeworms (Cestoda).

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of corn, soybeans and other legumes, rice, cereal (e.g., wheat, oats, barley, rye, rice, maize), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and other specialty crops (e.g., canola, sunflower, olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

A wavy line in a structure fragment denotes the attachment point of the fragment to the remainder of the molecule. For example, when the variable Q in Formula 1 is defined as Q-1, the wavy line bisecting the bond in the 5-position of the pyrazole Q-1 means that the pyrazole Q-1 is attached to the remainder of the structure of Formula 1 at said 5-position, as shown below.

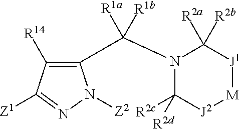

When the variable M is defined as —C(R^{3e})(A)-, this means that M is a carbon atom ring member substituted with one R^{3e} and one A as shown below.

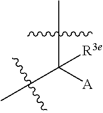

When Q is Q-2a or Q-2b and R^{14a} is H, the two imidazole tautomers are generally considered to be the same compound due to rapid interconversion as depicted below.

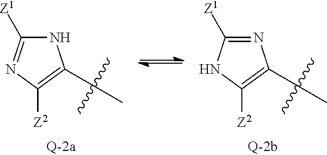

In the above recitations, the term “alkyl”, used either alone or in compound words such as “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. “Cycloalkenyl” includes groups such as cyclopentenyl and cyclohexenyl as well as groups with more than one double bond such as 1,3- and 1,4-cyclohexadienyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include CF₃, CH₂Cl, CH₂CF₃ and CCl₂CF₃.

The chemical abbreviations S(O) and S(═O) as used herein represent a sulfinyl moiety. The chemical abbreviations SO₂, S(O)₂ and S(═O)₂ as used herein represent a sulfonyl moiety. The chemical abbreviations C(O) and C(═O) as used herein represent a carbonyl moiety. The chemical abbreviations CO₂, C(O)O and C(═O)O as used herein represent an oxycarbonyl moiety. “CHO” means formyl.

When A¹ is benzyl substituted with 1 to 3 R⁴, the R⁴ substituent(s) may be attached to any available carbon atom of the benzyl group (i.e. the R⁴ substituent(s) may be attached to the benzylic carbon atom or to any available carbon atom of the phenyl group).

The total number of carbon atoms in a substituent group is indicated by the “Cᵢ-Cⱼ” prefix where i and j are numbers from 1 to 8. For example, C₁-C₄ alkyl designates methyl through butyl.

When a group contains a substituent which can be hydrogen, for example R^{1a} or R^{1b}, then when this substituent is taken as hydrogen, it is recognized that this is equivalent to said group being unsubstituted. When a variable group is shown to be optionally attached to a position, for example (R^{v})ᵣ in U-36 of Exhibit 1 wherein r may be 0, then hydrogen can be at the position even if not recited in the variable group definition. When one or more positions on a group are said to be “not substituted” or “unsubstituted”, then hydrogen atoms are attached to take up any free valency.

Unless otherwise indicated, a “ring” or “ring system” as a component of Formula 1 is carbocyclic or heterocyclic. The term “ring system” denotes two or more connected rings. The term “bicyclic ring system” denotes a ring system consisting of two rings sharing two or more common atoms.

A ring or a bicyclic ring system can be part of an extended ring system containing more than two rings wherein substituents on the ring or bicyclic ring system are taken together to form the additional rings, which may be in bicyclic relationships with other rings in the extended ring system.

The term “aromatic” indicates that each of the ring atoms is essentially in the same plane and has a p-orbital perpendicular to the ring plane, and that (4n+2) π electrons, where n is a positive integer, are associated with the ring or ring system to comply with Hülckel's rule.

The terms “heterocyclic ring” or “heterocycle” denotes a ring wherein at least one of the atoms forming the ring backbone is other than carbon. Unless otherwise indicated, a heterocyclic ring can be a saturated, partially unsaturated, or fully unsaturated ring. “Saturated heterocyclic ring” refers to a heterocyclic ring containing only single bonds between ring members. “Partially saturated heterocyclic ring” refers a heterocyclic ring containing at least one double bond but which is not aromatic. The term “heteroaromatic ring” denotes a fully unsaturated aromatic ring in which at least one atom forming the ring backbone is not carbon. Typically a heteroaromatic ring contains no more than 4 nitrogens, no more than 1 oxygen and no more than 1 sulfur. Unless otherwise indicated, heteroaromatic rings can be attached through any available carbon or nitrogen by replacement of a hydrogen on said carbon or nitrogen. The term “heteroaromatic bicyclic ring system” denotes a ring system consisting of two fused rings, in which at least one of the two rings is a heteroaromatic ring as defined above.

When a radical is optionally substituted with listed substituents with the number of substituents stated (e.g., “up to 5”), then the radical may be unsubstituted or substituted with a number of substituents ranging up to the high number stated (e.g., “5”), and the attached substituents are independently selected from the substituents listed.

When a substituent is a ring or ring system, it can be attached to the remainder of Formula 1 through any available ring member, unless otherwise described.

The term “unsubstituted” in connection with a group such as a ring or ring system means the group does not have any substituents other than its one or more attachments to the remainder of Formula 1.

The phrase “phenyl or a 5- or 6-membered heteroaromatic ring, each ring unsubstituted or substituted with 1 to 3 R⁴” means that each phenyl ring is unsubstituted or substituted with 1 to 3 R⁴, each 5-membered heteroaromatic ring is unsubstituted or substituted with 1 to 3 R⁴, and each 6-membered heteroaromatic ring is unsubstituted or substituted with 1 to 3 R⁴.

When the number of optional substituents is not restricted by an expressed limitation (e.g., the phrase “unsubstituted or substituted with at least one substituent independently selected from”), it is understood to mean that the number of optional substituents can range from 0 up to the number of positions available. One skilled in the art will appreciate that while some substituents such as halogen can be present at every available position (for example, the C₂F₅ substituent is a C₂ alkyl group substituted with the maximum number of 5 fluorine atoms), practical factors such as cost and synthetic accessibility can limit the number of occurrences of other substituents. These limitations are part of the general synthetic knowledge known to those skilled in the art. Of note are embodiments wherein in the absence of expressed limitation of number of optional substituents, the number of optional substituents is up to 3 (i.e. 0, 1, 2 or 3) if accommodated by the number of available positions.

As noted above, substituents such as A can be (among others) a 5- or 6-membered heteroaromatic ring, optionally substituted with one or more substituents selected from a group of substituents as defined in the Summary of Invention. Examples of a 5- or 6-membered heteroaromatic ring optionally substituted with one or more substituents include the rings U-2 through U-61 illustrated in Exhibit 1 wherein R^{v} is any substituent as defined in the Summary of the Invention (e.g., for R¹) and r is an integer from 0 to 5, limited by the number of available positions on each U group. As U-29, U-30, U-36, U-37, U-38, U-39, U-40, U-41, U-42 and U-43 have only one available position, for these U groups r is limited to the integers 0 or 1, and r being 0 means that the U group is unsubstituted and a hydrogen is present at the position indicated by (R^{v})ᵣ.

### Exhibit 1

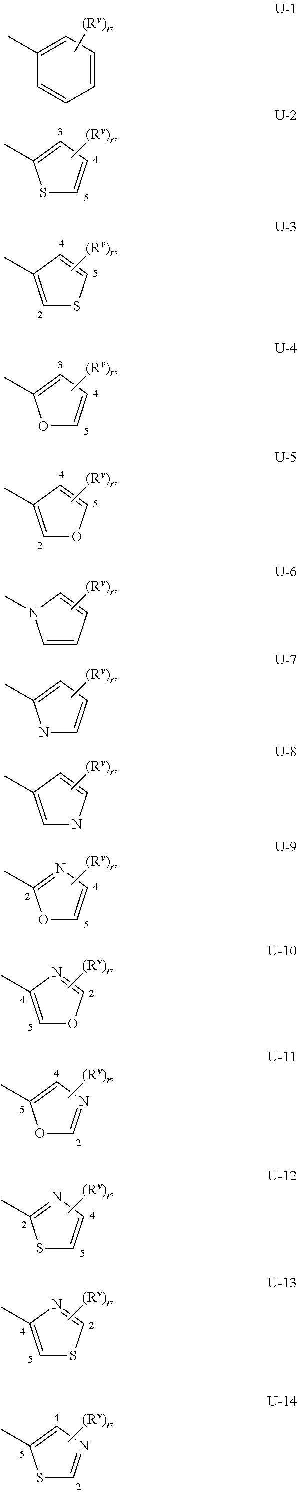

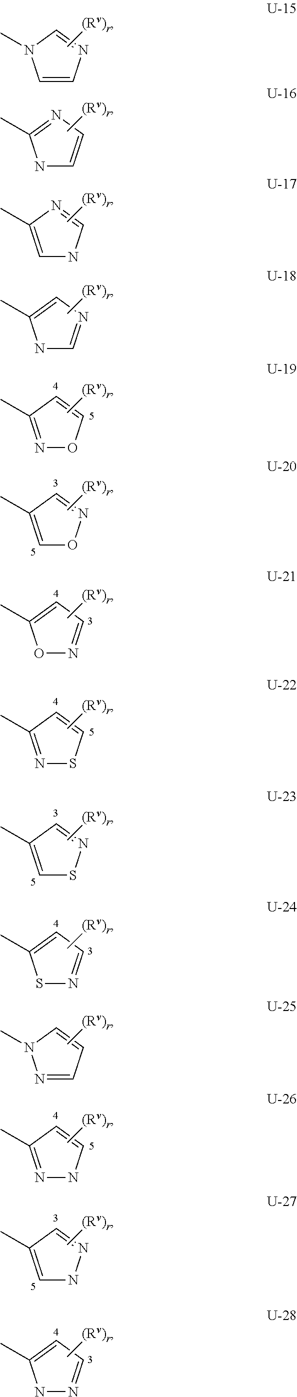

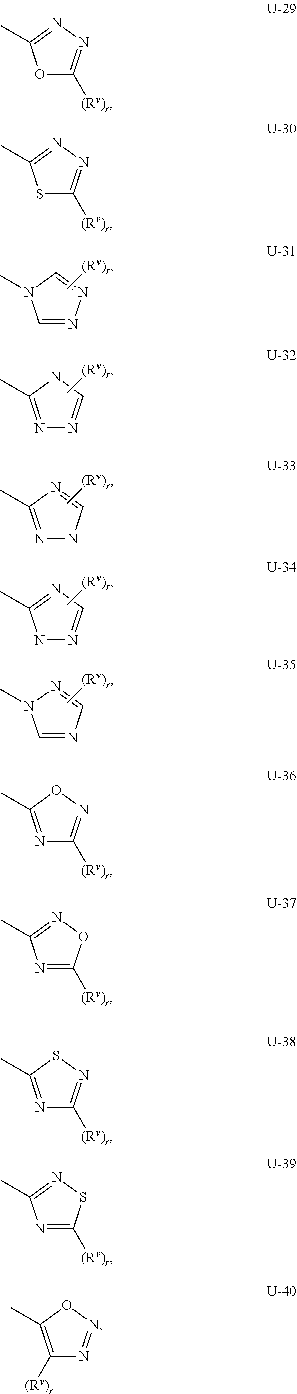

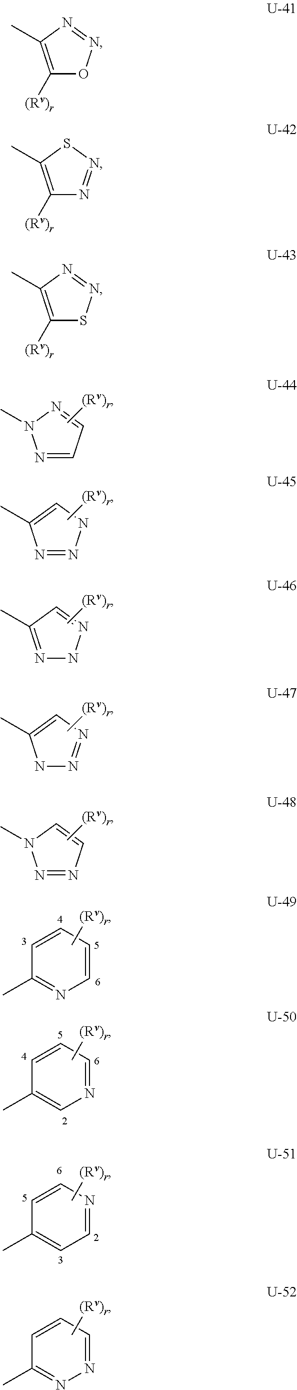

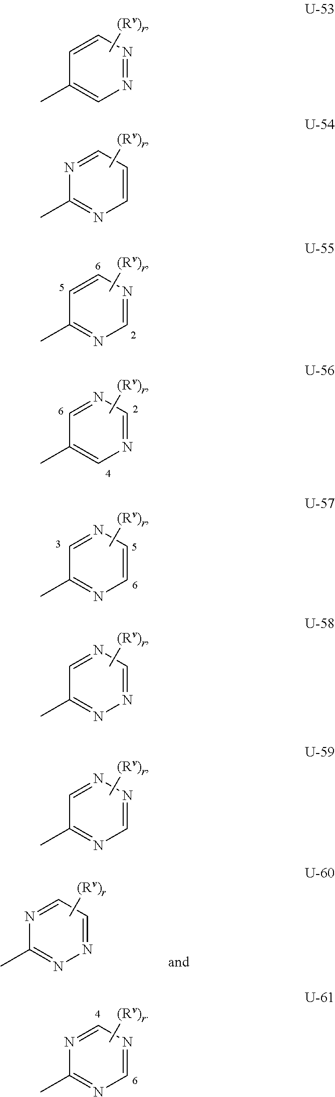

As noted above, substituents such as A can be (among others) a 7-, 8-, 9-, 10- or 11-membered heteroaromatic bicyclic ring system optionally substituted with up to 3 substituents selected from a group of substituents as defined in the Summary of Invention. Examples of an 8-, 9- or 10-membered heteroaromatic bicyclic ring system optionally substituted with up to 3 substituents include the ring systems H-1 through H-23 illustrated in Exhibit 2 wherein R^{v} is any substituent as defined in the Summary of the Invention (e.g., for A) and r is an integer from 0 to 3, limited by the number of available positions on each H group.

### Exhibit 2

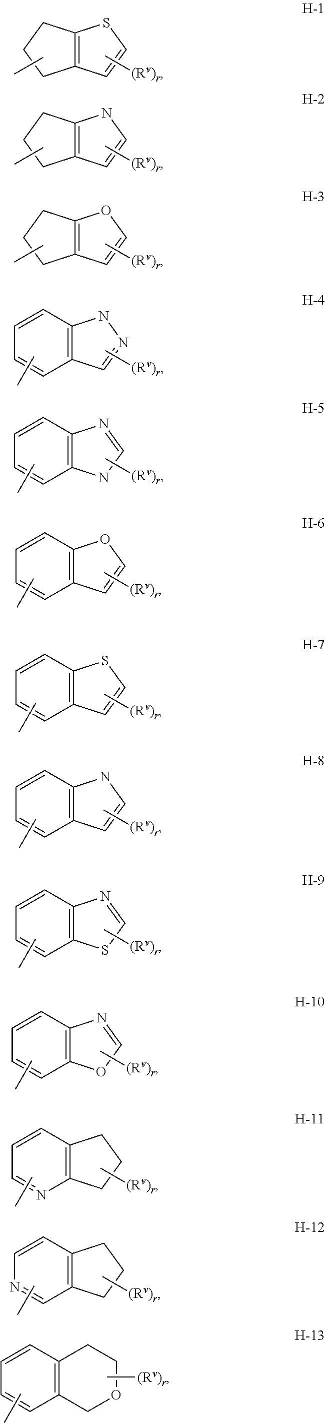

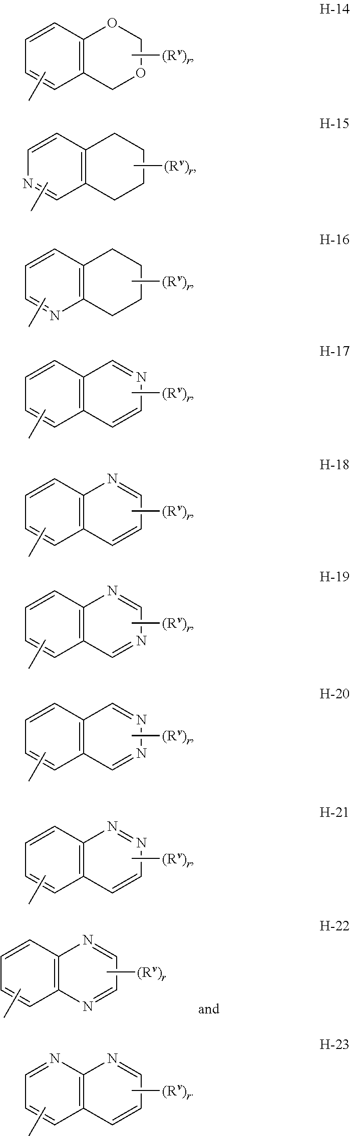

Although R^{v} groups are shown in the structures U-1 through U-61 and H-1 through H-23, it is noted that they do not need to be present since they are optional substituents. The nitrogen atoms that require substitution to fill their valence are substituted with H or R^{v}. Note that when the attachment point between (R^{v})ᵣ and the U or H group is illustrated as floating, (R^{v})ᵣ can be attached to any available carbon atom or nitrogen atom of the U or H group. Note that when the attachment point on the U or H group is illustrated as floating, the U or H group can be attached to the remainder of Formula 1 through any available carbon or nitrogen of the U or H group by replacement of a hydrogen atom.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

Compounds of this invention can exist as one or more conformational isomers due to restricted bond rotation caused by steric hinderance. This invention comprises mixtures of conformational isomers. In addition, this invention includes compounds that are enriched in one conformer relative to others.

Compounds selected from Formula 1 (including all stereoisomers, N-oxides, and salts thereof) typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and 3-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of invertebrate pests. The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention relates to compounds selected from Formula 1, N-oxides, and salts thereof.

Embodiments of the present invention as described in the Summary of the Invention include those described below. In the following Embodiments Formula 1 includes stereoisomers, N-oxides, and salts thereof, and reference to “a compound of Formula 1” includes the definitions of substituents specified in the Summary of the Invention unless further defined in the Embodiments.

### Embodiment 1

A compound of Formula 1 wherein R^{1a} is H, cyano, C₁-C₆ alkyl, C₃-C₆ cycloalkyl, C₁-C₆ haloalkyl, C(O)OH, C(O)R^{5a}, C(O)OR^{6a} or C(O)NR^{7a}R^{8a}.

### Embodiment 1a

A compound of Embodiment 1 wherein R^{1a} is H, cyano or C₁-C₆ alkyl.

### Embodiment 1b

A compound of Embodiment 1a wherein R^{1a} is H, cyano or methyl.

### Embodiment 1c

A compound of Embodiment 1b wherein R₁ₐ is H.

### Embodiment 2

A compound of Formula 1 or any one of Embodiments 1 through 1c wherein R^{1b} is H or methyl.

### Embodiment 2a

A compound of Embodiment 2 wherein R^{1b} is H.

### Embodiment 2b

A compound of Formula 1 wherein R^{1a} is H and R^{1b} is H.

### Embodiment 2c

A compound of Formula 1 wherein R^{1a}, R^{1b}, R^{2a}, R^{2b}, R^{2c} and R^{2d} are H.

### Embodiment 3

A compound of Formula 1 wherein M is —C(R^{3e})A)- or O.

### Embodiment 3a

A compound of Embodiment 3 wherein M is —C(R^{3e})(A)-.

### Embodiment 3b

A compound of Embodiment 3 wherein M is O.

### Embodiment 3c

A compound of Formula 1 or any one of Embodiments 1 through 3a wherein A is cyano, C(X)R⁵, C(O)OR⁶, C(X)NR⁷R⁸ or NR⁹R¹⁰; or C₁-C₆ alkyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl, C(O)R^{5a}, C(O)OR^{6a}, C(O)NR^{7a}R^{8a} and S(O)ₙR^{11a}; or phenyl or a 5- or 6-membered heteroaromatic ring, each ring unsubstituted or substituted with 1 to 3 R⁴.

### Embodiment 3d

A compound of Embodiment 3c wherein A is cyano, C(X)R⁵, C(O)OR⁶ or NR⁹R¹⁰; or phenyl or a 5- or 6-membered heteroaromatic ring, each ring unsubstituted or substituted with 1 to 3 R⁴.

### Embodiment 3e

A compound of Embodiment 3d wherein A is cyano, C(X)R⁵, C(O)OR⁶, NR⁹R¹⁰ or 1,3,4-oxadiazole.

### Embodiment 3f

A compound of Embodiment 3e wherein A is cyano, C(O)OR^{6a}, NHC(O)R^{5a}, NHC(O)OR^{6a} or 1,3,4-oxadiazole.

### Embodiment 3f1

A compound of Embodiment 3f wherein A is cyano or NHC(O)Me.

### Embodiment 3g

A compound of Embodiment 3d wherein A is a 5- or 6-membered heteroaromatic ring, each ring unsubstituted or substituted with 1 to 3 R⁴.

### Embodiment 3h

A compound of Embodiment 3g wherein A is a 5-membered heteroaromatic ring, unsubstituted or substituted with 1 to 3 R⁴.

### Embodiment 3i

A compound of Embodiment 3h wherein A is 1,3,4-oxadiazole.

### Embodiment 3j

A compound of Embodiment 3g wherein A is a 6-membered heteroaromatic ring, unsubstituted or substituted with 1 to 3 R⁴.

### Embodiment 3k

A compound of Embodiment 3j wherein A is a pyridine ring, unsubstituted or substituted with 1 to 3 R⁴.

### Embodiment 3l

A compound of Embodiment 3k wherein A is 4-pyridinyl, unsubstituted or substituted with 1 to 3 R⁴.

### Embodiment 4

A compound of Formula 1 or any one of Embodiments 1 through 3l wherein each R^{2a}, R^{2b}, R^{2c} and R^{2d} is independently H, cyano, C₁-C₆ alkyl, C(O)OR⁶, NHC(O)R^{5a} or NHC(O)OR^{6a}.

### Embodiment 4a

A compound of Embodiment 4 wherein each R^{2a}, R^{2b}, R^{2c} and R^{2d} is independently H or C₁-C₆ alkyl.

### Embodiment 4b

A compound of Embodiment 4a wherein each R^{2a}, R^{2b}, R^{2c} and R^{2d} is independently H or methyl.

### Embodiment 4c

A compound of Formula 1 or Embodiment 4b wherein R^{2a}, R^{2b}, R^{2c} and R^{2d} are H.

### Embodiment 4d

A compound of Formula 1 wherein R^{1a}, R^{1b}, R^{2a}, R^{2b}, R^{2c} and R^{2d} are H.

### Embodiment 5

A compound of Formula 1 or any one of Embodiments 1 through 4d wherein each R^{3a}, R^{3b}, R^{3c} and R^{3d} is H, halogen or C₁-C₆ alkyl.

### Embodiment 5a

A compound of Embodiment 5 wherein each R^{3a}, R^{3b}, R^{3c} and R^{3d} is H, F, Cl, Br or methyl.

### Embodiment 5b

A compound of Embodiment 5a wherein R^{3a}, R^{3b}, R^{3c} and R^{3d} are H.

### Embodiment 5c

A compound of Formula 1 wherein R^{1a}, R^{1b}, R^{2a}, R^{2b}, R^{2c}, R^{2d}, R^{3a}, R^{3b}, R^{3c} and R^{3d} are H.

### Embodiment 5d

A compound of Formula 1 wherein R^{3e} is H, or R^{3e} and A can be taken together to form a 5- or 6-membered ring containing a —C(═O)NR⁷— amide group.

### Embodiment 5e

A compound of Formula 1 wherein R^{3e} is H.

### Embodiment 5f

A compound of Formula 1 wherein J¹ is —C(R^{3a}R^{3b})— and J² is a direct bond or —C(R^{3c}R^{3d})—.

### Embodiment 5g

A compound of Embodiment 5f wherein J¹ is —C(R^{3a}R^{3b})— and J² is —C(R^{3c}R^{3d})—.

### Embodiment 5h

A compound of Embodiment 5f wherein J¹ is —C(R^{3a}R^{3b})— and J² is a direct bond.

### Embodiment 6

A compound of Formula 1 or any one of Embodiments 1 through 5h wherein Z¹ is phenyl substituted with 1 to 4 R^{4a}.

### Embodiment 6a

A compound of Formula 1 or any one of Embodiments 1 through 5h wherein Z¹ is a 5-membered heteroaromatic ring, unsubstituted or substituted with 1 to 3 R^{4a}.

### Embodiment 6b

A compound of Embodiment 6a wherein Z¹ is furanyl, thienyl, oxazolyl or thiazolyl, each unsubstituted or substituted with 1 to 3 R^{4a}.

### Embodiment 6c

A compound of Formula 1 or any one of Embodiments 1 through 5b wherein Z¹ is a 6-membered heteroaromatic ring, unsubstituted or substituted with 1 to 3 R^{4a}.

### Embodiment 6d

A compound of Embodiment 6c wherein Z¹ is pyridinyl or pyrimidinyl, each unsubstituted or substituted with 1 to 3 R^{4a}.

### Embodiment 6e

A compound of Embodiment 6d wherein Z¹ is pyridinyl, unsubstituted or substituted with 1 to 3 R^{4a}.

### Embodiment 6f

A compound of Embodiment 6e wherein Z¹ is 2-pyridinyl, unsubstituted or substituted with halogen.

### Embodiment 6g

A compound of Formula 1 wherein Z¹ is phenyl and one R^{4a} is in the 3- or 4-position.

### Embodiment 6h

A compound of Embodiment 6g wherein the one R^{4a} in the 3- or 4-position is halogen, C₁-C₆ alkyl, C₁-C₆ haloalkyl or C₁-C₆ haloalkyloxy.

### Embodiment 7

A compound of Formula 1 or any one of Embodiments 1 through 5e wherein Z² is phenyl, unsubstituted or substituted with 1 to 4 R^{4b}.

### Embodiment 7a

A compound of Formula 1 or any one of Embodiments 1 through 5e wherein Z² is phenyl substituted with 1 to 4 R^{4b}.

### Embodiment 7b

A compound of Formula 1 or any one of Embodiments 1 through 5e wherein Z² is a 5-membered heteroaromatic ring, unsubstituted or substituted with 1 to 3 R^{4b}.

### Embodiment 7c

A compound of Embodiment 7b wherein Z² is furanyl, thienyl, oxazolyl or thiazolyl, each unsubstituted or substituted with 1 to 3 R^{4b}.

### Embodiment 7d

A compound of Formula 1 or any one of Embodiments 1 through 5e wherein Z² is a 6-membered heteroaromatic ring, unsubstituted or substituted with 1 to 3 R^{4b}.

### Embodiment 7e

A compound of Embodiment 7d wherein Z² is pyridinyl or pyrimidinyl, each unsubstituted or substituted with 1 to 3 R^{4b}.

### Embodiment 7f

A compound of Embodiment 7e wherein Z² is pyridinyl, unsubstituted or substituted with 1 to 3 R^{4b}.

### Embodiment 7g

A compound of Embodiment 7f wherein Z² is 2-pyridinyl, unsubstituted or substituted with halogen.

### Embodiment 7h

A compound of Formula 1 wherein Z² is phenyl and one R^{4b} is in the 2-, 4- or 6-position.

### Embodiment 7i

A compound of Embodiment 7h wherein the one R^{4b} in the 2-, 4- or 6-position is halogen or methyl.

### Embodiment 8

A compound of Formula 1 wherein each R^{4b} is independently halogen, cyano, C₁-C₆ alkyl, C₁-C₆ haloalkyl or C₁-C₆ haloalkyloxy.

### Embodiment 9

A compound of Formula 1 or any one of Embodiments 1 through 8 wherein Q is Q-1.

### Embodiment 9a

A compound of Embodiment 9 wherein Q is Q-1 and R¹⁴ is hydrogen.

### Embodiment 10a

A compound of Formula 1 or any one of Embodiments 1 through 8 wherein Q is Q-2a or Q-2b.

### Embodiment 10b

A compound of Embodiment 10a wherein Q is Q-2a.

### Embodiment 10c

A compound of Embodiment 10a wherein Q is Q-2b.

### Embodiment 10d

A compound of Embodiment 10a wherein Q is Q-2a or Q-2b and R^{14a} is H.

### Embodiment 11

A compound of Formula 1 or any one of Embodiments 1 through 8 wherein Q is Q-3.

### Embodiment 12

A compound of Formula 1 or any one of Embodiments 1 through 8 wherein Q is Q-4.

### Embodiment 13

A compound of Formula 1 or any one of Embodiments 1 through 8 wherein Q is Q-1, Q-2a or Q-2b.

### Embodiment 14

A compound of Formula 1 or any one of Embodiments 1 through 8 wherein Q is Q-1, Q-2a, Q-2b or Q-3.

### Embodiment 14a

A compound of Formula 1 or any one of Embodiments 1 through 8 wherein Q is Q-2a, Q-2b or Q-3.

### Embodiment 15

A compound of Formula 1 or any one of Embodiments 1 through 14 wherein X is O.

### Embodiment 15a

A compound of Formula 1 or any one of Embodiments 1 through 14 wherein X is S.

### Embodiment S1

A compound of Formula 1 wherein
   R^{1a}, R^{1b}, R^{2a}, R^{2b}, R^{2c} and R^{2d} are H;
   J¹ is —C(R^{3a}R^{3b})—;
   J² is a direct bond or —C(R^{3c}R^{3d})—;
   R^{3a}, R^{3b}, R^{3c} and R^{3d} are H;
   Q is Q-2a, Q-2b or Q-3;
   Z¹ is phenyl substituted with 1 to 4 R^{4a}; or pyridinyl, unsubstituted or substituted with 1 to 3 R^{4a};
   Z² is phenyl, unsubstituted or substituted with 1 to 4 R^{4b}; or Z² is pyridinyl, unsubstituted or substituted with 1 to 3 R^{4b};
   M is —C(R^{3e})(A)-;
   R^{3e} is H; and
   A is cyano or NHC(O)Me.

Embodiments of this invention, including Embodiments 1-15a above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-15a above as well as any other embodiments described herein, and any combination thereof, pertain to the compositions and methods of the present invention.

Combinations of Embodiments 1-15a are illustrated by:

### Embodiment A

A compound of Formula 1 wherein
   R^{1a} is H; and
   R^{1b} is H.

### Embodiment B

A compound of Embodiment A wherein
   J¹ is a direct bond or —C(R^{3a}R^{3b})—; and
   J² is a direct bond.

### Embodiment C.

A compound of Embodiment B wherein
   M is —C(R^{3e})(A)- or —O—; and
   A is cyano, C(X)R⁵, C(O)OR⁶, C(X)NR⁷R⁸or NR⁹R¹⁰), S(O)ₙR¹¹ or SO₂NR⁹R¹⁰; or phenyl, a 5- or 6-membered heteroaromatic ring or a 7-to 11-membered heteroaromatic ring system, each ring or ring system unsubstituted or substituted with 1 to 3 R⁴.

### Embodiment D

A compound of Embodiment C wherein
   Q is Q-1, Q-2a or Q-2b.

### Embodiment E

A compound of Embodiment D wherein
   Z¹ is phenyl substituted with 1 to 4 R^{4a}; or pyridinyl, unsubstituted or substituted with 1 to 3 R^{4a}; and
   Z² is phenyl, unsubstituted or substituted with 1 to 4 R^{4b}; or Z² is pyridinyl, unsubstituted or substituted with 1 to 3 R^{4b}.

### Embodiment F.

A compound of Embodiment E wherein
   R^{2a}, R^{2b}, R^{2c} and R^{2d} are H;
   A is cyano, C(O)OR^{6a}, NR^{9a}C(O)R^{5a}, NHC(O)OR^{6a} or 1,3,4-oxadiazolyl; and
   R^{9a} is H or C₁-C₄ alkyl.

Combinations of Embodiments 1-15a are also illustrated by:

### Embodiment A1

A compound of Formula 1 wherein
   R^{1a}, R^{1b}, R^{2a}, R^{2b}, R^{2c} and R^{2d} are H.

### Embodiment B1

A compound of Embodiment A1 wherein
   J¹ is —C(R^{3a}R^{3b})—;
   J² is a direct bond or —C(R^{3c}R^{3d})—; and
   R^{3a}, R^{3b}, R^{3c} and R^{3d} are H.

### Embodiment C1

A compound of Embodiment B1 wherein
   M is —C(R^{3e})(A)- or —O—; and
A is cyano, C(X)R⁵, C(O)OR⁶, C(X)NR⁷R⁸or NR⁹R⁸, S(O)ₙR¹¹ or SO₂NR⁹R¹⁰; or phenyl, or a 5- or 6-membered heteroaromatic ring, each ring unsubstituted or substituted with 1 to 3 R⁴.

### Embodiment D1

A compound of Embodiment C1 wherein
   Q is Q-2a, Q-2b or Q-3.

### Embodiment E1

A compound of Embodiment D1 wherein
   Z¹ is phenyl substituted with 1 to 4 R^{4a}; or pyridinyl, unsubstituted or substituted with 1 to 3 R^{4a}; and
   Z² is phenyl, unsubstituted or substituted with 1 to 4 R^{4b}; or Z² is pyridinyl, unsubstituted or substituted with 1 to 3 R^{4b}.

### Embodiment F1

A compound of Embodiment E1 wherein
   A is cyano, C(O)OR^{6a}, NR^{9a}C(O)R^{5a}, NHC(O)OR^{6a} or 1,3,4-oxadiazolyl; and
   R^{9a} is H or C₁-C₄ alkyl.

### Embodiment G1

A compound of Embodiment F1 wherein
   M is —C(R^{3e})(A)-;
   R^{3e} is H; and
   A is cyano or NHC(O)Me.

Specific embodiments include compounds of Formula 1 (compound numbers refer to compounds in Index Tables A-N) selected from the group consisting of compound numbers 12, 14, 20, 22, 23, 24, 36, 37, 44, 57 and 65.

Specific embodiments also include compounds of Formula 1 (compound numbers refer to compounds in Index Tables A-N) selected from the group consisting of compound numbers 36, 86, 90, 100, 112, 113, 119, 136, 137, 138, 141 and 152.

Of note is that compounds of this invention are characterized by favorable metabolic and/or soil residual patterns and exhibit activity controlling a spectrum of agronomic and nonagronomic invertebrate pests.

Of particular note, for reasons of invertebrate pest control spectrum and economic importance, protection of agronomic crops from damage or injury caused by invertebrate pests by controlling invertebrate pests are embodiments of the invention. Compounds of this invention because of their favorable translocation properties or systemicity in plants also protect foliar or other plant parts which are not directly contacted with a compound of Formula 1 or a composition comprising the compound.

Also noteworthy as embodiments of the present invention are compositions comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent.

Further noteworthy as embodiments of the present invention are compositions for controlling an invertebrate pest comprising a compound (i.e. in a biologically effective amount) of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent (i.e. in a biologically effective amount).

Embodiments of the invention further include methods for controlling an invertebrate pest comprising contacting the invertebrate pest or its environment with a biologically effective amount of a compound of any of the preceding Embodiments (e.g., as a composition described herein).

Embodiments of the invention also include a composition comprising a compound of any of the preceding Embodiments in the form of a soil drench liquid formulation. Embodiments of the invention further include methods for controlling an invertebrate pest comprising contacting the soil with a liquid composition as a soil drench comprising a biologically effective amount of a compound of any of the preceding Embodiments.

Embodiments of the invention also include a spray composition for controlling an invertebrate pest comprising a compound (i.e. in a biologically effective amount) of any of the preceding Embodiments and a propellant. Embodiments of the invention further include a bait composition for controlling an invertebrate pest comprising a compound (i.e. in a biologically effective amount) of any of the preceding Embodiments, one or more food materials, optionally an attractant, and optionally a humectant. Embodiments of the invention also include a device for controlling an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

Embodiments of the invention also include a method for protecting a seed from an invertebrate pest comprising contacting the seed with a biologically effective amount of a compound of any of the preceding Embodiments (e.g., as a composition described herein).

Embodiments of the invention also include methods for protecting an animal from an invertebrate parasitic pest comprising administering to the animal a parasiticidally effective amount of a compound of any of the preceding Embodiments.

Embodiments of the invention also include methods for controlling an invertebrate pest comprising contacting the invertebrate pest or its environment with a biologically effective amount of a compound of Formula 1, an N-oxide, or a salt thereof, (e.g., as a composition described herein), provided that the methods are not methods of medical treatment of a human or animal body by therapy.

This invention also relates to such methods wherein the invertebrate pest or its environment is contacted with a composition comprising a biologically effective amount of a compound of Formula 1, an N-oxide, or a salt thereof, and at least one additional component selected from the group consisting of surfactants, solid diluents and liquid diluents, said composition optionally further comprising a biologically effective amount of at least one additional biologically active compound or agent, provided that the methods are not methods of medical treatment of a human or animal body by therapy.

One or more of the following methods and variations as described in Schemes 1-16 can be used to prepare the compounds of Formula 1. The definitions of R^{1a}, R^{1b}, R^{2a}, R^{2b}, R^{2c}, R^{2d}, R¹⁴, Z¹, Z², J¹, J² and M in the compounds of Formulae 1-24 below are as defined above in the Summary of the Invention unless otherwise noted. Ambient or room temperature is defined as about 20-25° C.

Compounds of Formula 1a (compounds of Formula 1 wherein Q is Q-1) can be prepared by cycloaddition of compounds of Formula 2 with compounds of Formula 3 as shown in Scheme 1. This method involves heating the reactants from about 25 to about 150° C. in an inert organic solvent such as t-butanol in the presence of a base such as triethylamine (see, for example, Broggini, Gianluigi et al. Synthesis 1996, (9), pages 1076-1078).

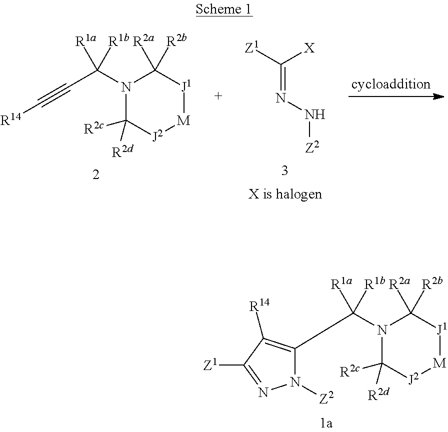

Compounds of Formula 2 can be prepared by the method shown in Scheme 2. In this method, an appropriately substituted amine of Formula 4 is treated with a base such as potassium carbonate in an inert organic solvent such as acetone, followed by the addition of the propargyl halide of Formula 5 to yield the alkyne of Formula 2. Compounds of Formulae 4 and 5 are either commercially available or can be prepared by well-established methods known in the art.

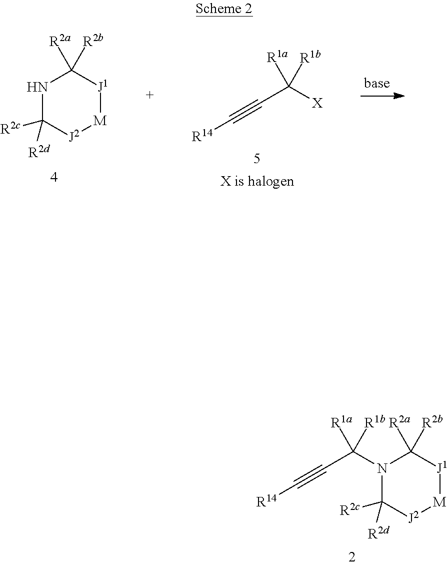

Compounds of Formula 3 can be prepared by halogenation of compounds of Formula 6 as shown in Scheme 3. Typical reaction conditions for this method include an inert organic solvent such as dichloromethane, a halogenating reagent such as a N-halosuccinimide, and an optional activating agent such as dimethyl sulfide (see, for example, Dadiboyena, Sureshbabu et al. Tetrahedron Letters 2009, 50(3), pages 291-294). Typical reaction temperatures range from −78 to 100° C. Alternatively, compounds of Formula 3 can be prepared using CuCl₂ (see, for example, Attanasi, Orazio et al. Canadian Journal of Chemistry 1983, 61(12), pages 2665-2668).

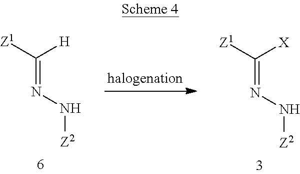

Compounds of Formula 6 are prepared by the condensation of a hydrazine of Formula 7 with an aldehyde of Formula 8 as shown in Scheme 4. Typical reaction conditions include heating the reactants to a temperature from about 25 to about 110° C. for up to several hours in an organic solvent such as ethanol (see, for example, Raghav, N. et al. Journal of Chemical and Pharmaceutical Research 2010, 2(4), pages 801-807). Compounds of Formulae 7 and 8 are either commercially available or can be prepared by well-established methods known in the art.

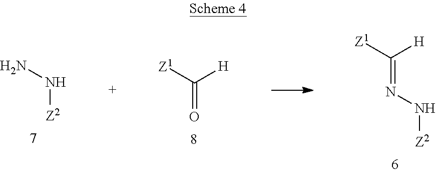

Compounds of Formula 1b (compounds of Formula 1 wherein Q is Q-2a or Q-2b and R^{14a} is H) can be prepared by reaction of amines of Formula 4, paraformaldehyde, and imidazoles of Formula 9 in a variety of solvents such as acetic acid and methanol and reaction temperatures from 25 to 100° C. (see, for example, Sanghani, Sunil G. et al. Archives of Applied Science Research 2010, 2(5), pages 444-450). This method is shown in Scheme 5.

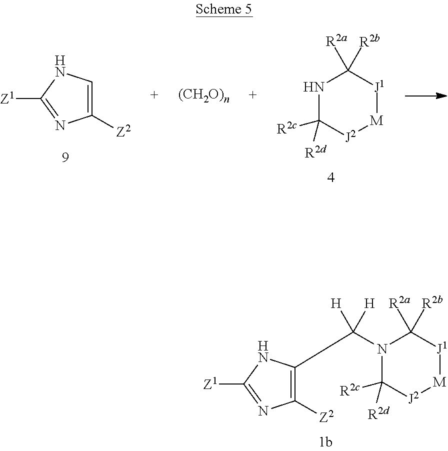

Compounds of Formulae 9 can be prepared by reaction of aldehydes of Formula 8 with glyoxyals of Formula 10 in the presence of ammonium acetate. The reaction is typically carried out at room temperature in a polar solvent such as methanol (see, for example, Selig, Roland et al. Tetrahedron 2011, 67(47), pages 9204-9213). This method is shown in Scheme 6. Compounds of Formulae 8 and 10 are either commercially available or can be prepared by well-established methods known in the art.

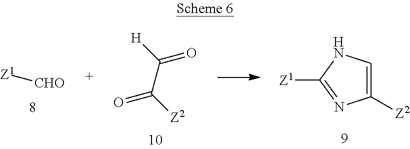

Compounds of Formula 1b can be alkylated in the presence of a base such as potassium carbonate or sodium hydride in an organic solvent such as tetrahydrofuran with alkylating agents such as iodomethane or other haloalkyls to give compounds of Formulae 1b-1 and 1b-2 (see, for example Li, Ziyong et al. Dyes and Pigments 2011, 90(3), pages 290-296). This method is shown in Scheme 7.

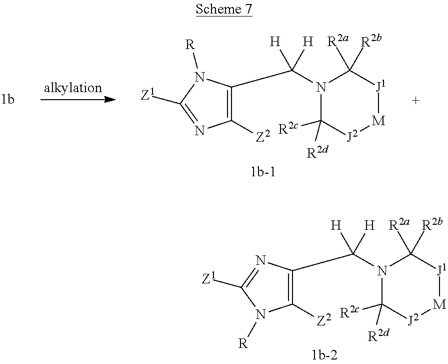

Compounds of Formula 1c (compounds of Formula 1 wherein Q is Q-3) can be prepared as shown in Scheme 8 by reductive amination of aldehydes of Formula 11. In this method, aldehydes of Formula 11 are condensed with amines of Formula 4, followed by reduction with a mild reducing agent such as sodium triacetoxy borohydride in a solvent such as dichloromethane (see, for example, Li, J. et al. Bioorganic & Medicinal Chemistry Letters 2010, 20(16), pages 4932-4935).

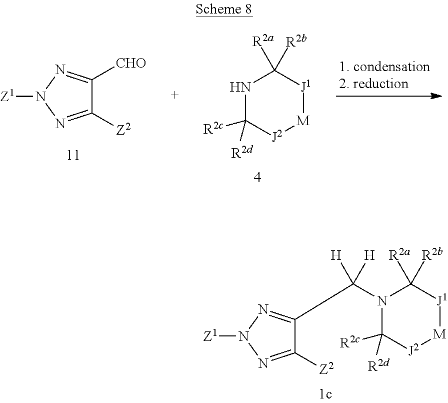

Aldehydes of Formula 11 can be prepared from esters of Formula 13 by a variety of methods known in the art. In the example shown in Scheme 9, reduction of esters of Formula 13 provides alcohols of Formula 12. A typical reduction method includes a reducing agent such as lithium aluminum hydride in an anhydrous organic solvent such as tetrahydrofuran (see, for example, Koike, Tatsuki et al. Journal of Medicinal Chemistry 2011, 54(12), pages 4207-4218). The alcohols of Formula 12 can then be oxidized to the aldehydes of Formula 11 by a number of methods known in the art. Examples of such oxidations include oxidation with manganese dioxide (Elsner, Jan et al. Journal of Medicinal Chemistry 2005, 48(18), pages 5771-5779), oxidation with pyridinium chlorochromate (Menozzi, Giulia et al. Bioorganic and Medicinal Chemistry 2004, 12(20), pages 5465-5483) and Swern oxidation (Omura, K. et al. Tetrahedron 1978, 34, page 1651).

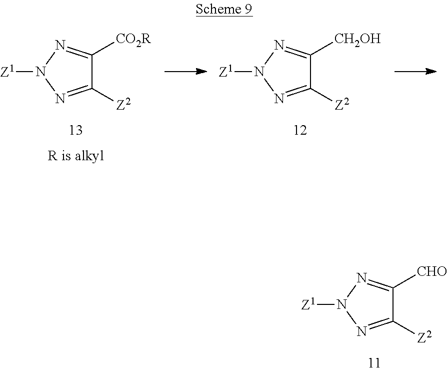

Compounds of Formula 13 are prepared by reaction of hydrazinylidines of Formula 14 with ammonium acetate in the presence of copper (IT) chloride in a solvent such as ethanol at temperatures ranging from 25 to 125° C. (see, for example, El Sekily, Mohamed A. et al. Journal of Chemical Research 2006, 12, pages 771-773). This method is shown in Scheme 10.

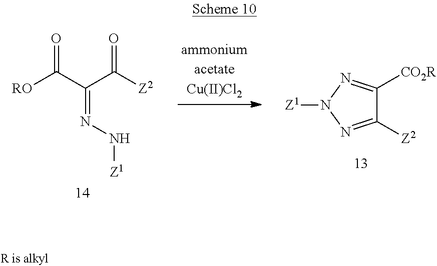

Compounds of Formula 14 are prepared as shown in Scheme 11. An amine of Formula 15 is treated with sodium nitrite in an aqueous solution of hydrochloric and acetic acids at 0° C., followed by addition of this solution to a reaction mixture of a compound of Formula 16 in the presence of a base such as sodium acetate or sodium bicarbonate in an organic solvent such as ethanol (see, for example, El Sekily, Mohamed A. et al. Journal of Chemical Research 2006, 12, pages 771-773). Compounds of Formulae 15 and 16 are either commercially available or can be prepared by well-established methods know in the art.

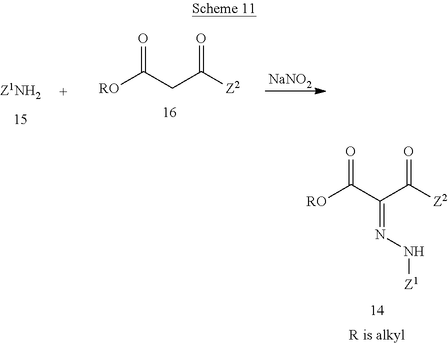

Compounds of Formula 1d (compounds of Formula 1 wherein Q is Q-4) can be prepared by reaction of compounds of Formula 17 with compounds of Formula 18 in the presence of a metal catalyst such as PdCl₂ or CuI, optionally in the presence of a ligand, and a base such as silver carbonate or cesium carbonate. Typical reaction solvents include water or N,N-dimethylformamide, and typical reaction temperatures are from 25 to 200° C. (see, for example, Derridj, Fazia et al. Journal of Organometallic Chemistry 2008, 693(1), pages 135-144 or Ohnmacht, Stephan A. et al. Chemical Communications 2008, 10, pages 1241-1243). Compounds of Formula 18 are either commercially available or can be prepared by well-established methods known in the art. This method is shown in Scheme 12.

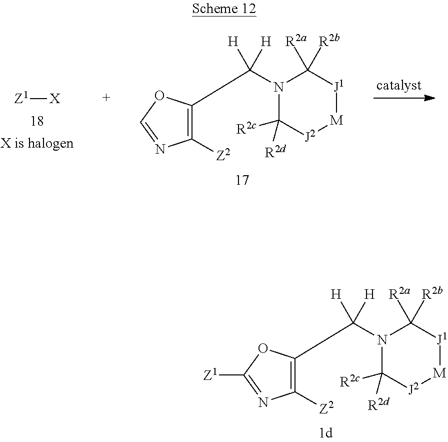

Compounds of Formula 17 can be prepared by treatment of compounds of Formula 19 with amine compounds of Formula 4. Typical reaction solvents include tetrahydrofuran or acetonitrile. Addition of a base such as potassium carbonate is often advantageous. Typical reaction temperatures range from 25 to 100° C. (see, for example, Boulos, John et al. Journal of Heterocyclic Chemistry 2006 43(2), pages 443-445). This method is shown in Scheme 13.

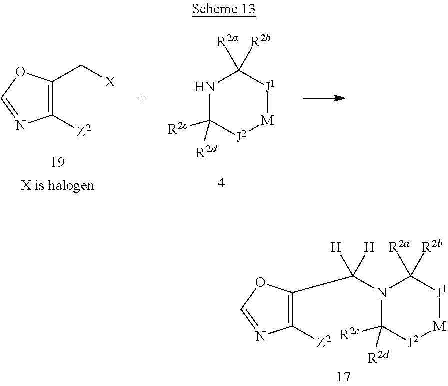

Halides of Formula 19 can be prepared from alcohols of Formula 20 by various methods known in the art, including treatment of the alcohol with carbon tetrabromide or phosphorous oxychloride (see, for example, Boulos, John et al. Journal of Heterocyclic Chemistry 2006 43(2), pages 443-445). Alcohols of Formula 20 can be prepared by the reduction of esters of Formula 21 with a variety of reducing agents such as lithium aluminum hydride in tetrahydrofuran (see, for example, Boulos, John et al. Journal of Heterocyclic Chemistry 2006 43(2), pages 443-445). These methods are shown in Scheme 14.

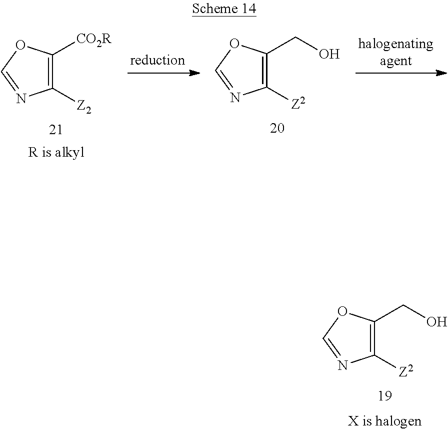

Compounds of Formula 21 can be prepared from compounds of Formula 22 by reaction with an alkyl glyoxalate in an anhydrous organic solvent such as tetrahydrofuran (see, for example, Sisko, Joseph et al. Journal of Organic Chemistry 2000, 65(5), pages 1516-1524). This method is shown in Scheme 15.

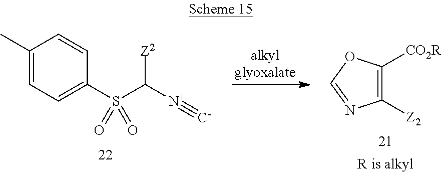

Compounds of Formula 22 can be prepared from compounds of Formula 23 as shown in Scheme 16. In this method, an aldehyde of Formula 23 is first treated with formamide in the presence of chlorotrimethylsilane in an organic solvent such as toluene or acetonitrile, followed by treatment with p-toluenesulfinic acid to provide the intermediate of Formula 24. The compound of Formula 24 is then treated with phosphorous oxychloride in an inert solvent such as tetrahydrofuran, followed by dehydrohalogenation with a base such as 2,6-lutidine, to yield the isocyanide of Formula 22 (see, for example, Sisko, Joseph et al. Journal of Organic Chemistry 2000, 65(5), pages 1516-1524).

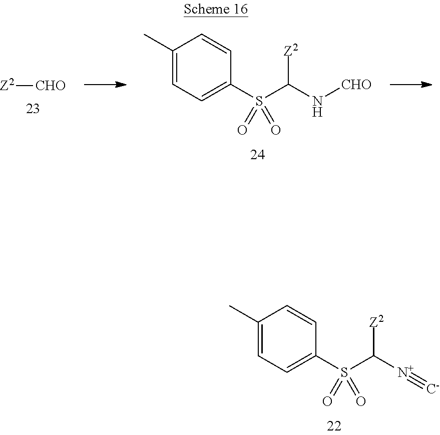

Compounds of Formula 1 containing the groups C(X)R⁵, C(X)R^{5a}, C(X)NR⁷R^{8a} and C(X)NR^{7a}R^{8a} wherein X is S can be prepared from corresponding compounds of Formula 1 wherein X is O by general methods known in the art involving treatment with thionating reagents such as P₄S₁₀ or Lawessen's Reagent (2,4-bis(4-methoxyphenyl)-1,3-dithia-2,4-diphosphetane 2,4-disulfide).

Schemes 1 through 16 illustrate methods to prepare compounds of Formula 1 having a variety of substituents. Compounds of Formula 1 having substituents other than those particularly noted for Schemes 1 through 16 can be prepared by general methods known in the art of synthetic organic chemistry, including methods analogous to those described for Schemes 1 to 16.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Synthesis Examples are, therefore, to be construed as merely illustrative, and not limiting of the disclosure in any way whatsoever. Steps in the following Synthesis Examples illustrate a procedure for each step in an overall synthetic transformation, and the starting material for each step may not have necessarily been prepared by a particular preparative run whose procedure is described in other Examples or Steps. Ambient or room temperature is defined as about 20-25° C. Percentages are by weight except for chromatographic solvent mixtures or where otherwise indicated. Parts and percentages for chromatographic solvent mixtures are by volume unless otherwise indicated. MPLC refers to medium pressure liquid chromatography on silica gel. ¹H NMR spectra are reported in ppm downfield from tetramethylsilane; “s” means singlet, “d” means doublet, “dd” means doublet of doublets, “ddd” means doublet of doublet of doublets, “t” means triplet, “m” means multiplet, and “br s” means broad singlet. Compound numbers refer to compounds in Index Tables A-N.

### SYNTHESIS EXAMPLE 1

### Preparation of methyl 1-[[1-(4-fluorophenyl)-3-[3-(trifluoromethyl)phenyl]-1H-pyrazol-5-yl]methyl]-4-piperidinecarboxylate (compound number 6)

### Step A: Preparation of 3-(trifluoromethyl)benzaldehyde 2-(4-fluorophenyl)hydrazone

A slurry of 4-fluorophenylhydrazine hydrochloride (1.6 g, 10 mmol), 3-(trifluoromethyl)benzaldehyde (1.64 g, 10 mmol), and sodium acetate (0.83 g, 10 mmol) in ethanol (22 mL) was stirred at room temperature for 18 hours. The reaction mixture was concentrated under reduced pressure to remove ethanol, and ether (120 mL) and water (50 mL) were added. The ether solution was separated, dried over anhydrous magnesium sulfate, and concentrated under reduced pressure to provide 2.53 g of the title compound which was used in the next step without further purification.

### Step B: Preparation of [C—(Z)]—N-(4-fluorophenyl)-3-(trifluoromethyl)benzenecarbohydrazonoyl chloride

3-(Trifluoromethyl)benzaldehyde 2-(4-fluorophenyl)hydrazone (8.04 g) was dissolved in N,N-dimethylformamide (40 mL), and the solution was warmed to 35° C. N-chlorosuccinimide (3.4 g) was added to the solution over 5 minutes, maintaining the temperature at 40-58° C. The red solution was then stirred for 15 minutes and poured into a mixture of 400 g of ice and water. After stirring for 1.5 hours, the red solid was collected by filtration and dried to give 6.9 g of the title compound which was used in the next step without further purification.

### Step C: Preparation of methyl 1-(2-propyn-1-yl)-4-piperidinecarboxylate

To a solution of methyl isonipecotate (7 g, 49 mmol) and triethylamine (10.2 mL) in dichloromethane (200 mL) cooled in a water ice bath was added a solution of propargyl bromide (13 mL, 80% w/w in toluene). The resulting slurry was stirred at room temperature for 40 hours, after which time water (70 mL) was added. The aqueous and organic phases were separated, and the aqueous solution was extracted with dichloromethane (2×50 mL). The combined organic layers were dried over anhydrous magnesium sulfate, filtered, and concentrated in vacuo to yield a semi-solid, which was extracted with ether (40 mL). The ether solution was concentrated under reduced pressure to yield the title compound as a yellow oil (1.8 g). ¹H NMR (CDCl₁): δ 3.61 (s, 3H); 3.23 (s, 2H), 2.79 (br d, 2H), 2.20 (m, 4H), 1.87 (br d, 2H), 1.73 (m, 2H).

### Step D: Preparation of methyl 1-[[1-(4-fluorophenyl)-3-[3-(trifluoromethyl)phenyl]-1H-pyrazol-5-yl]methyl]-4-piperidinecarboxylate

A solution of [C—(Z)]—N-(4-fluorophenyl)-3-(trifluoromethyl)benzenecarbohydrazonoyl chloride (968 mg), methyl 1-(2-propyn-1-yl)-4-piperidinecarboxylate (495 mg) and triethylamine (2 mL) in t-butanol (5 mL) was heated at 85° C. for 24 hours. Additional triethylamine (0.5 mL) was added, and heating was continued for another 24 hours. Ethyl acetate (80 mL) and water (20 mL) were added to the reaction mixture, and the separated organic layer was washed with water (20 mL), filtered through a Varian Chem Elut® filter, and concentrated in vacuo to a crude oil. This crude oil was purified by MPLC (silica gel eluted with 0-35% ethyl acetate in hexane) to give the title compound, a compound of this invention, as a thick oil (200 mg). ¹H NMR (CDCl₃): δ 8.11 (s, 1H), 8.05 (d, 1H), 7.80 (m, 2H), 7.55 (m, 2H), 7.17 (t, 2H), 6.69 (s, 1H), 3.69 (s, 3H), 3.43 (s, 2H), 2.90 (br d, 2H), 2.30 (m, 1H), 2.10 (m, 2H), 1.90 (m, 2H), 2.70 (m, 2H).

### SYNTHESIS EXAMPLE 2

### Preparation of 4-[[5-(2,4-difluorophenyl)-2-[3-(trifluoromethyl)phenyl]-1H-imidazol-4-yl]methyl]morpholine (compound number 44), 4-[[4-(2,4-difluorophenyl)-1-methyl-2-[3-(trifluoromethyl)phenyl]-1H-imidazol-5-yl]methyl]morpholine (compound number 45), and 4-[[5-(2,4-difluorophenyl)-1-methyl-2-[3-(trifluoromethyl)phenyl]-1l-imidazol-4-yl]methyl]morpholine (compound number 49)

### Step A: Preparation of 4-(2,4-difluorophenyl)-2-[3-(trifluoromethyl)phenyl]-1H-imidazole

A solution of 2,4-difluorophenylglyoxal in methanol (25 mL) was added dropwise over 10 minutes to a solution of 3-(trifluoromethyl)benzaldehyde (0.70 mL, 5.0 mmol) and ammonium acetate (1.95 g, 25.2 mmol) in methanol (25 mL), and the reaction mixture was stirred at 23° C. for 18 hours. The reaction mixture was then concentrated under reduced pressure, and the residue was adsorbed onto silica gel and purified by column chromatography, eluting with 0-100% ethyl acetate/hexanes, to afford the title compound as a colorless solid (0.805 g, 49%). ¹H NMR (CDCl₃): δ 8.97 (br s, 1H), 8.13-8.06 (m, 2H), 8.06-8.03 (m, 1H), 7.61-7.58 (m, 1H), 7.55-7.51 (m, 1H), 7.50 (d, 1H), 6.96 (dddd, 1H), 6.88 (ddd, 1H).

### Step B: Preparation of 4-[[5-(2,4-difluorophenyl)-2-[3-(trifluoromethyl)phenyl]-1H-imidazol-4-yl]methyl]morpholine

4-(2,4-Difluorophenyl)-2-[3-(trifluoromethyl)phenyl]-1H-imidazole (0.20 g, 0.6 mmol), paraformaldehyde (0.11 g, 1.2 mmol), and morpholine (0.06 g, 0.7 mmol) were combined in acetic acid (0.5 mL) and methanol (1.0 mL) in a 40 mL scintillation vial, and the reaction mixture was heated to 70° C. for 19 hours. The solvent was removed under reduced pressure, and the residue was partitioned between 1 M NaOH (100 mL) and ethyl acetate (30 mL). The phases were separated, and the aqueous phase was extracted with ethyl acetate (30 mL). The combined organic layers were dried over magnesium sulfate, filtered, and concentrated under reduced pressure. The residue was purified by column chromatography on silica gel, eluting with 0-100% ethyl acetate/hexanes, to afford the product, a compound of this invention, as a colorless solid. (0.21 g, 78%). The product exists as a 4:1 mixture of two imidazole tautomers which do not equilibrate on the NMR time scale. ¹H NMR (CDCl₃) (major tautomer): δ 9.91 (br s, 1H), 8.13 (s, 1H), 8.05 (d, 1H), 7.70-7.54 (m, 3H), 7.05-6.86 (m, 2H), 3.75-3.70 (m, 4H), 3.58 (s, 2H), 2.51-2.45 (m, 4H); (minor tautomer): δ 9.85 (br s, 1H), 8.11 (s, 1H), 8.04 (d, 1H), 7.70-7.54 (m, 3H), 7.05-6.86 (m, 2H), 3.70-3.66 (m, 4H), 3.58 (s, 2H), 2.58-2.53 (m, 4H).

### Step C: Preparation of 4-[[4-(2,4-difluorophenyl)-1-methyl-2-[3-(trifluoromethyl)phenyl]-1H-imidazol-5-yl]methyl]morpholine and 4-[[5-(2,4-difluorophenyl)-1-methyl-2-[3-(trifluoromethyl)phenyl]-1H-imidazol-4-yl]methyl]morpholine

A 20 mL scintillation vial was charged with sodium hydride (60% oil dispersion, 0.02 g, 0.4 mmol) and anhydrous tetrahydrofuran (3.0 mL). 4-[[5-(2,4-Difluorophenyl)-2-[3-(trifluoromethyl)phenyl]-1H-imidazol-4-yl]methyl]morpholine (0.15 g, 0.4 mmol) was added, and the reaction mixture was stirred at 23° C. until the cloudy suspension became homogeneous (about 5 minutes). Iodomethane (0.02 mL, 0.4 mmol) was then added, and the reaction mixture was stirred at 23° C. for 18 hours. The reaction mixture was adsorbed onto silica gel and purified by column chromatography, eluting with 0-100% ethyl acetate/hexanes to obtain 4-[[4-(2,4-difluorophenyl)-1-methyl-2-[3-(trifluoromethyl)phenyl]-1H-imidazol-5-yl]methyl]morpholine (isomer A, 0.04 g, 0.1 mmol, 26%), a compound of this invention, as a yellow solid, and then with 0-10% methanol/dichloromethane to obtain 4-[[5-(2,4-difluorophenyl)-1-methyl-2-[3-(trifluoromethyl)phenyl]-1H-imidazol-4-yl]methyl]morpholine, a compound of this invention, as a yellow oil (isomer B, 0.07 g, 0.2 mmol, 42%). ¹H NMR (CDCl₃) (isomer A): δ 7.96 (s, 1H), 7.87 (d, 1H), 7.68 (d, 1H), 7.60 (t, 1H), 7.54-7.49 (m, 1H), 6.97-6.92 (m, 1H), 6.91-6.87 (m, 1H), 3.83 (s, 3H), 3.66-3.62 (m, 4H), 3.54 (s, 2H), 2.38-2.33 (m, 4H); (isomer B): δ 7.97 (s, 1H), 7.88 (d, 1H), 7.68 (d, 1H), 7.60 (t, 1H), 7.50-7.45 (m, 1H), 7.04-6.97 (m, 2H), 3.66-3.62 (m, 4H), 3.51 (s, 3H), 3.43 (bs, 2H), 2.48-2.38 (m, 4H).

### SYNTHESIS EXAMPLE 3

### Preparation of 1-[[2-(3,4-dichlorophenyl)-5-(2-fluorophenyl)-2H-1,2,3-triazol-4-yl]methyl]-4-piperidinecarbonitrile (compound number 51)

### Step A: Preparation of ethyl α-[2-(3,4-dichlorophenyl)hydrazinylidine]-2-fluoro-β-oxobenzenepropanoate

To a 0° C. solution of 3,4-dichloroaniline (200 mg, 1.23 mmol) in acetic acid (1.0 mL), water (1.0 mL) and concentrated HCl (0.2 mL) was added sodium nitrite (102 mg) in water, and the reaction mixture was stirred at 0° C. for 30 minutes. The resultant solution was slowly added to a stirred solution of ethyl 2-fluoro-β-oxo-benzenepropanoate (260 mg, 1.23 mmol), sodium carbonate (285 mg, 2.70 mmol) and sodium acetate (222 mg, 2.70 mmol) in ethanol (15 mL), and the mixture was allowed to stir at room temperature for 2 hours. The reaction mixture was diluted with water (50 mL) and extracted with ethyl acetate (3×10 mL). The combined organic extracts were washed with water and brine, then dried over anhydrous sodium sulfate and concentrated under reduced pressure to afford the title compound. ¹H NMR (300 MHz, CDCl₃): δ 12.74 (br s, 1H), 7.67 (m, 1H), 7.52 (m, 2H), 7.34 (d, 1H, J=8.7 Hz), 7.28-7.21 (m, 2H), 7.11 (m, 2H), 6.93 (dd, 1H, J=8.81 Hz), 4.38 (q, 2H), 1.34 (t, 3H).

### Step B: Preparation of ethyl 2-(3,4-dichlorophenyl)-5-(2-fluorophenyl)-2H-1,2,3-triazole-4-carboxylate

To a stirred solution of ethyl α-[2-(3,4-dichlorophenyl)hydrazinylidine]-2-fluoro-β-oxobenzenepropanoate (471 mg, 1.23 mmol) in ethanol (5 mL) was added Cu(II)Cl₂ (363 mg, 2.7 mmol) and ammonium acetate (950 mg, 12.3 mmol), and the reaction mixture was refluxed for 12 hours under a nitrogen atmosphere. The reaction mixture was poured into ice-cold 6N HCl (10 mL) and extracted with ethyl acetate (3×10 mL). The combined organic extracts were washed with water and brine, then dried over anhydrous sodium sulfate and concentrated under reduced pressure. Purification of the crude material by silica-gel chromatography (1:9 ethyl acetate:hexanes) afforded the title compound (230 mg, 48%) as an off-white solid. ¹H NMR (300 MHz, CDCl₃): δ 8.34 (d, 1H, J=2.51 Hz), 8.01 (d, 1H, J=8.9 Hz), 7.63-7.57 (m, 2H), 7.56-7.44 (m, 1H), 7.30-7.16 (m, 2H), 4.38 (q, 2H), 1.29 (t, 3H).

### Step C: Preparation of 2-(3,4-dichlorophenyl)-5-(2-fluorophenyl)-2H-1,2,3-triazole-4-methanol

To a stirred solution of lithium aluminum hydride (0.60 mL, 2M solution in tetrahydrofuran, 1.2 mmol) was added ethyl 2-(3,4-dichlorophenyl)-5-(2-fluorophenyl)-2H-1,2,3-triazole-4-carboxylate (230 mg, 60 mmol) in tetrahydrofuran dropwise at 0° C.; the reaction mixture was stirred for 2 hours and then warmed to room temperature. The resulting mixture was quenched with saturated aqueous sodium sulfate solution and diluted with ethyl acetate (15 mL). The precipitated inorganic solid was removed by filtration, and the filtrate was washed with water and brine, then dried over anhydrous sodium sulfate and concentrated under reduced pressure to afford the title compound (180 mg, 87%). ¹H NMR (300 MHz, CDCl₃): δ 8.27 (d, 1H, J=2.57 Hz), 7.98 (d, 1H, J=8.86 Hz), 7.70 (m, 1H), 7.56 (d, 1H, J=8.78 Hz), 7.46 (m, 1H), 7.31-7.18 (m, 2H), 4.88 (d, 2H).

### Step D: Preparation of 2-(3,4-dichlorophenyl)-5-(2-fluorophenyl)-2H-1,2,3-triazole-4-carboxaldehyde

To a stirred solution of 2-(3,4-dichlorophenyl)-5-(2-fluorophenyl)-2H-1,2,3-triazole-4-methanol (210 mg, 0.66 mmol) in dichloromethane (5 mL) was added Dess-Martin periodinane reagent (337 mg, 0.79 mmol), and the reaction mixture was stirred at room temperature for 2 hours. The reaction mixture was diluted with dichloromethane (20 mL) and washed with aqueous 2N NaOH solution, water and brine, then dried over anhydrous sodium sulfate and concentrated under reduced pressure. The crude material was purified by silica-gel chromatography (1:9 ethyl acetate:hexanes) to afford the title compound (140 mg, 67%) as an off-white solid. ¹H NMR (300 MHz, CDCl₃): δ 10.24 (s, 1H), 8.36 (d, 1H, J=2.56 Hz), 8.07 (dd, 1H, J=8.81 Hz), 7.68 (m, 1H), 7.51 (m, 1H), 7.33-7.21 (m, 2H).

### Step E: Preparation of 1-[[2-(3,4-dichlorophenyl)-5-(2-fluorophenyl)-2H-1,2,3-triazol-4-yl]methyl]-4-piperidinecarbonitrile

To a stirred solution of 2-(3,4-dichlorophenyl)-5-(2-fluorophenyl)-2H-1,2,3-triazole-4-carboxaldehyde (140 mg, 0.41 mmol) in 1,2-dichloroethane (3 mL) was added 4-piperidinecarbonitrile (137 mg, 1.25 mmol), and the reaction mixture was stirred at room temperature for 1 hour. Sodium triacetoxyborohydride (147 mg, 1.25 mmol) was added, and the reaction mixture was further stirred at 50° C. for 3 hours. The reaction mixture was cooled to room temperature, diluted with dichloromethane and washed with water and brine, then dried over anhydrous sodium sulfate and concentrated under reduced pressure. Purification of the crude residue by C₁₈ reverse-phase column chromatography afforded the title compound, a compound of this invention as a white solid (84 mg, 53%) melting at 113-116° C. ¹H NMR (300 MHz, CDCl₃): δ 8.25 (d, 1H, J=2.56 Hz), 7.97 (dd, 1H, J=8.78 Hz), 7.64 (m, 1H), 7.54 (d, 1H, J=8.7 Hz), 7.45 (m, 1H), 7.28-7.15 (m, 2H), 3.75 (s, 1H), 2.70-2.61 (m, 2H), 2.59-2.50 (m, 1H), 2.39-2.29 (m, 2H), 1.85-1.63 (m, 4H).

### SYNTHESIS EXAMPLE 4

### Preparation of N-[1-[[4-(4-fluorophenyl)-2-[3-(trifluoromethyl)phenyl]-5-oxazolyl]methyl]-4-piperidinyl]acetamide (compound number 65)

### Step A: Preparation of N-[(4-fluorophenyl)[(4-methylphenyl)sulfonyl]methyl]formamide

To a solution of 4-fluorobenzaldehyde (14.2 g, 100 mmol) in a mixture of toluene (50 mL) and acetonitrile (50 mL) was added formamide (9.93 mL, 250 mmol) and chlorotrimethylsilane (14.0 mL, 110 mmol), and the resultant mixture was stirred under a nitrogen atmosphere and heated at 50-55° C. for 7 hours. p-Toluenesulfinic acid (21.9 g, 140.0 mmol) was added, and the mixture was stirred and heated at 50° C. for 6 hours, and then 3 hours at ambient temperature.

Methyl tert-butyl ether (180 mL) and water (170 mL) were added, and the reaction mixture was stirred at ambient temperature for 20 minutes. The layers were separated, and the aqueous layer was extracted with methyl tert-butyl ether (50 mL). The combined organic phase was dried over sodium sulfate, and volatiles were removed under reduced pressure to obtain a solid residue. Hexanes (100 mL) and water (100 mL) were added to the solid residue, and the resulting slurry was stirred at room temperature for 30 minutes, then filtered. The filter cake was washed with hexanes (2×100 mL) and dried in a vacuum-oven at 30° C. overnight. The title compound was obtained as a white powder (20 g, 60%). ¹H NMR (CDCl₃, 400 MHz): δ 8.18 (s, 1H), 7.6 (d, 2H), 7.4 (d, 2H), 7.35 (d, 2H), 7.0 (d, 2H), 6.9 (br s, 1H), 6.27 (d, 1H), 2.43 (s, 3H).

### Step B: Preparation of 1-fluoro-4-[isocyano[(4-methylphenyl)sulfonyl]methyl]benzene

To a mixture of N-[(4-fluorophenyl)[(4-methylphenyl)sulfonyl]methyl]formamide (the product of Step A) (20.07 g, 63.6 mmol) and tetrahydrofuran (150 mL) was added phosphorous oxychloride (11.9 mL, 127 mmol) over a period of 5 minutes, and the resulting mixture was stirred at room temperature for 10 minutes. The reaction was then cooled using an ice/water bath to about 5° C., and 2,6-lutidine (44.5 mL, 382.0 mmol) was added dropwise over 30 minutes, maintaining the temperature at less than 12° C. The cooling bath was removed and the mixture was stirred at ambient temperature for 18 hours. The reaction mixture was poured into a stirred, ice-water cooled mixture of ice and saturated aqueous sodium bicarbonate. This mixture was extracted twice with ethyl acetate, about 100 mL each time. The combined organic extracts were washed with 10% aqueous hydrochloric acid, saturated aqueous sodium bicarbonate, and brine, and were then dried over sodium sulfate and concentrated under reduced pressure. Isopropyl alcohol (about 180 mL) and water (about 90 mL) were added to the residual solid, and the resulting slurry was stirred at room temperature for 2 hours. The slurry was filtered, and the cake was washed twice with a mixture of 2:1 by volume of isopropyl alcohol-water (2×10 mL) and dried in a vacuum-oven at 30° C. for 48 hours. The title compound was obtained as a solid (10 g, 52%).

¹HNMR (CDCl₃, 400 MHz): δ 7.65 (d, 2H), 7.38 (d, 4H), 7.1 (d, 2H), 5.5 (s, 1H), 2.5 (s, 3H).

### Step C: Preparation of ethyl 4-(4-fluorophenyl)oxazole-5-carboxylate

To a solution of 1-fluoro-4-[isocyano[(4-methylphenyl)sulfonyl]methyl]benzene (the product of Step B) (10 g, 34.36 mmol) in tetrahydrofuran (100 mL) was added ethyl glyoxalate (4.2 g as 50% solution in toluene, 41.2 mmol) and potassium carbonate solid (18.4 g, 137.4 mmol). The resulting mixture was stirred at room temperature for 16 hours. The reaction mixture was poured into ice cold water and extracted with ethyl acetate (2×200 mL). The combined organic extracts were washed with brine solution, dried over sodium sulfate and concentrated under reduced pressure. The residue thus obtained was purified by column chromatography on silica gel (60-120 mesh) eluted with a solvent gradient of 10 to 15% ethyl acetate/hexanes to obtain the desired product as a tan solid (4 g, 50%). ¹H NMR (CDCl₃, 400 MHz): δ 8.2 (d, 2H), 7.2 (d, 2H), 4.4 (q, 2H), 1.4 (t, 3H).

### Step D: Preparation of 4-(4-fluorophenyl)-5-(hydroxymethyl)oxazole

To a solution of ethyl 4-(4-fluorophenyl)oxazole-5-carboxylate (the product of Step C) (4 g, 17 mmol) in anhydrous tetrahydrofuran (40 mL) under a nitrogen atmosphere was added lithium tetrahydroaluminate (1.29 g, 34.0 mmol). The resulting mixture was stirred at 0 to 10° C. for 1 hour. The reaction mixture was poured into cold, saturated aqueous ammonium chloride solution and extracted with ethyl acetate (2×100 mL). The combined organic extracts were washed with brine solution, dried over sodium sulfate and concentrated under reduced pressure. The residue was purified by column chromatography on silica gel (60-120 mesh) eluted with a solvent gradient of 20 to 25% of ethyl acetate/hexane to afford the desired compound as a brown solid (2 g, 62%). ¹H NMR (CDCl₃, 400 MHz): δ 7.9 (s, 1H), 7.65 (m, 2H), 7.15 (m, 2H), 4.85 (s, 2H), 1.8 (s, 1H).

### Step E: Preparation of 5-(chloromethyl)-4-(4-fluorophenyl)oxazole

To a solution of 4-(4-fluorophenyl)-5-(hydroxymethyl)oxazole (the product of Step D) (2 g, 10.3 mmol) in N,N-dimethylformamide (20 mL) was added phosphorus oxychloride (2.38 g, 15.5 mmol) and the resulting mixture was stirred at ambient temperature for 6 hours. The reaction mixture was poured into ice cold water and extracted with ethyl acetate (2×40 mL). The combined organic extracts are washed with brine solution, dried over sodium sulfate and concentrated under reduced pressure. The residue was purified by column chromatography on silica gel (60-120 mesh) eluted with a solvent gradient of 15 to 20% ethyl acetate/hexane to afford the title compound as an oil (1.2 g, 46%). ¹H NMR (CDCl₃, 400 MHz): δ 7.95 (s, 1H), 7.75 (t, 2H), 7.2 (t, 2H), 4.9 (s, 2H).

### Step F: Preparation of N-[1-[[4-(4-fluorophenyl)-5-oxazolyl]methyl]-4-piperidinyl]acetamide

To a solution of 5-(chloromethyl)-4-(4-fluorophenyl)oxazole (the product of Step E) (1 g, 4.7 mmol) in acetonitrile (20 mL) was added 4-acetamidopiperidine (1.48 g, 7.05 mmol) and solid potassium carbonate (1.9 g, 14 mmol). The resulting mixture was stirred at 80° C. for 16 hours. The reaction mixture was poured into ice cold water and extracted with ethyl acetate (2×30 mL). The combined organic extracts were washed with brine solution, dried over sodium sulfate and concentrated under reduced pressure. The residue was purified by column chromatography on silica gel (60-120 mesh) eluted with a solvent gradient of 20 to 30% of ethyl acetate/hexane to afford the title compound as an off-white solid (800 mg, 61%). ¹H NMR (CDCl₃, 400 MHz): δ 8.2 (s, 1H), 7.8 (t, 2H), 7.2 (t, 2H), 3.8 (s, 2H), 2.9 (d, 2H), 2.2 (t, 2H), 1.9 (s, 3H), 1.8 (d, 2H) 1.5 (q, 2H).

### Step G: Preparation of N-[1-[[4-(4-fluorophenyl)-2-[3-(trifluoromethyl)phenyl]-5-oxazolyl]methyl]-4-piperidinyl]acetamide

To a mixture of N-[1-[[4-(4-fluorophenyl)-5-oxazolyl]methyl]-4-piperidinyl]acetamide (the product of Step F) (200 mg, 0.701 mmol) in water (2 mL) was added silver carbonate (386 mg, 1.40 mmol), 3-iodobenzotrifluoride (286 mg, 1.05 mmol), dichloro[1,1′-bis(diphenylphosphino)ferrocene]palladium(II)dichloromethane adduct (9.1 mg, 0.05 mmol), and triphenylphosphine (25 mg, 0.05 mmol). The resulting reaction mixture was stirred at 70° C. for 16 hours under a nitrogen atmosphere. The reaction mixture was poured into stirred, ice cold water and extracted with ethyl acetate (2×15 mL). The combined organic extracts were washed with brine solution, dried over sodium sulfate and concentrated under reduced pressure. The residue was purified by preparative thin layer chromatography (30% ethyl acetate/hexanes) to afford the title compound, a compound of this invention, as an off-white solid (100 mg, 33%). ¹H NMR (CDCl₁₃, 400 MHz): δ 8.39 (d, 2H), 7.9 (t, 3H), 7.19 (t, 1H), 7.2 (t, 2H) 3.9 (s, 2H), 3.65 (m, 1H), 2.95 (d, 2H), 2.3 (t, 2H), 1.9 (s, 3H), 1.8 (d, 2H) 1.5 (q, 2H).

By the procedures described herein together with methods known in the art, the following compounds of Tables 1 to 17 can be prepared. The following abbreviations are used in Tables 1 to 17 which follow: Me means methyl and Et means ethyl. R^{4a} and R^{4b} represent one or a combination of substituents.

TABLE 1

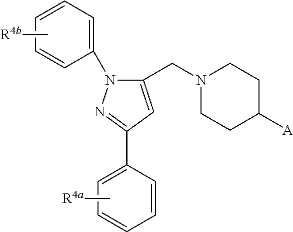

| | |
|---|---|
| R^{4b} | R^{4a} |

A is cyano

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 3-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is NHC(O)Me

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

TABLE 2

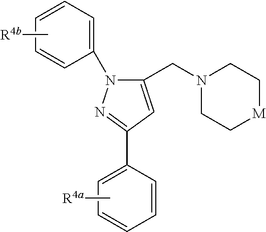

M is O

| R^{4b} | R^{4a} |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

TABLE 3

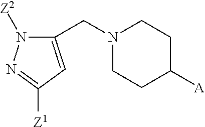

| | |
|---|---|
| Z¹ | Z² |

A is cyano

| | |
|---|---|
| 5-chloro-2-pyridinyl | 2-fluorophenyl |
| 5-chloro-2-pyridinyl | 2-chlorophenyl |
| 5-chloro-2-pyridinyl | 2-bromophenyl |
| 5-chloro-2-pyridinyl | 2-methylphenyl |
| 5-chloro-2-pyridinyl | 4-fluorophenyl |
| 5-chloro-2-pyridinyl | 2,4-difluorophenyl |
| 5-chloro-2-pyridinyl | 2,6-difluorophenyl |
| 5-chloro-2-pyridinyl | 2,4,6-trifluorophenyl |
| 5-(CF₃)-2-pyridinyl | 2-fluorophenyl |
| 5-(CF₃)-2-pyridinyl | 2-chlorophenyl |
| 5-(CF₃)-2-pyridinyl | 2-bromophenyl |
| 5-(CF₃)-2-pyridinyl | 2-methylphenyl |
| 5-(CF₃)-2-pyridinyl | 4-fluorophenyl |
| 5-(CF₃)-2-pyridinyl | 2,4-difluorophenyl |
| 5-(CF₃)-2-pyridinyl | 2,6-difluorophenyl |
| 5-(CF₃)-2-pyridinyl | 2,4,6-trifluorophenyl |
| 6-chloro-3-pyridazinyl | 2-fluorophenyl |
| 6-chloro-3-pyridazinyl | 2-chlorophenyl |
| 6-chloro-3-pyridazinyl | 2-bromophenyl |
| 6-chloro-3-pyridazinyl | 2-methylphenyl |
| 6-chloro-3-pyridazinyl | 4-fluorophenyl |
| 6-chloro-3-pyridazinyl | 2,4-difluorophenyl |
| 6-chloro-3-pyridazinyl | 2,6-difluorophenyl |
| 6-chloro-3-pyridazinyl | 2,4,6-trifluorophenyl |
| 4-(CF₃)-2-pyrimidinyl | 2-fluorophenyl |
| 4-(CF₃)-2-pyrimidinyl | 2-chlorophenyl |
| 4-(CF₃)-2-pyrimidinyl | 2-bromophenyl |
| 4-(CF₃)-2-pyrimidinyl | 2-methylphenyl |
| 4-(CF₃)-2-pyrimidinyl | 4-fluorophenyl |
| 4-(CF₃)-2-pyrimidinyl | 2,4-difluorophenyl |
| 4-(CF₃)-2-pyrimidinyl | 2,6-difluorophenyl |
| 4-(CF₃)-2-pyrimidinyl | 2,4,6-trifluorophenyl |
| 4-Cl-5-F-2-pyrimidinyl | 2-fluorophenyl |
| 4-Cl-5-F-2-pyrimidinyl | 2-chlorophenyl |
| 4-Cl-5-F-2-pyrimidinyl | 2-bromophenyl |
| 4-Cl-5-F-2-pyrimidinyl | 2-methylphenyl |
| 4-Cl-5-F-2-pyrimidinyl | 4-fluorophenyl |
| 4-Cl-5-F-2-pyrimidinyl | 2,4-difluorophenyl |
| 4-Cl-5-F-2-pyrimidinyl | 2,6-difluorophenyl |
| 4-Cl-5-F-2-pyrimidinyl | 2,4,6-trifluorophenyl |
| 5-bromo-2-pyridinyl | 2-fluorophenyl |
| 5-bromo-2-pyridinyl | 2-chlorophenyl |
| 5-bromo-2-pyridinyl | 2-bromophenyl |
| 5-bromo-2-pyridinyl | 2-methylphenyl |
| 5-bromo-2-pyridinyl | 4-fluorophenyl |
| 5-bromo-2-pyridinyl | 2,4-difluorophenyl |
| 5-bromo-2-pyridinyl | 2,6-difluorophenyl |
| 5-bromo-2-pyridinyl | 2,4,6-trifluorophenyl |
| 4,5-dichloro-2-pyridinyl | 2-fluorophenyl |
| 4,5-dichloro-2-pyridinyl | 2-chlorophenyl |
| 4,5-dichloro-2-pyridinyl | 2-bromoophenyl |
| 4,5-dichloro-2-pyridinyl | 2-methylphenyl |
| 4,5-dichloro-2-pyridinyl | 4-fluorophenyl |
| 4,5-dichloro-2-pyridinyl | 2,4-difluorophenyl |
| 4,5-dichloro-2-pyridinyl | 2,6-difluorophenyl |
| 4,5-dichloro-2-pyridinyl | 2,4,6-trifluorophenyl |
| 5-(CF₃)-2-pyrimidinyl | 2-fluorophenyl |
| 5-(CF₃)-2-pyrimidinyl | 2-chlorophenyl |
| 5-(CF₃)-2-pyrimidinyl | 2-bromophenyl |
| 5-(CF₃)-2-pyrimidinyl | 2-methylphenyl |
| 5-(CF₃)-2-pyrimidinyl | 4-fluorophenyl |
| 5-(CF₃)-2-pyrimidinyl | 2,4-difluorophenyl |
| 5-(CF₃)-2-pyrimidinyl | 2,6-difluorophenyl |
| 5-(CF₃)-2-pyrimidinyl | 2,4,6-trifluorophenyl |
| 4-Cl-5-(CF₃)-2-pyrimidinyl | 2-fluorophenyl |
| 4-Cl-5-(CF₃)-2-pyrimidinyl | 2-chlorophenyl |
| 4-Cl-5-(CF₃)-2-pyrimidinyl | 2-bromophenyl |
| 4-Cl-5-(CF₃)-2-pyrimidinyl | 2-methylphenyl |
| 4-Cl-5-(CF₃)-2-pyrimidinyl | 4-fluorophenyl |
| 4-Cl-5-(CF₃)-2-pyrimidinyl | 2,4-difluorophenyl |
| 4-Cl-5-(CF₃)-2-pyrimidinyl | 2,6-difluorophenyl |
| 4-Cl-5-(CF₃)-2-pyrimidinyl | 2,4,6-trifluorophenyl |
| 4-F-5-(CF₃)-2-pyrimidinyl | 2-fluorophenyl |
| 4-F-5-(CF₃)-2-pyrimidinyl | 2-chlorophenyl |
| 4-F-5-(CF₃)-2-pyrimidinyl | 2-bromophenyl |
| 4-F-5-(CF₃)-2-pyrimidinyl | 2-methylphenyl |
| 4-F-5-(CF₃)-2-pyrimidinyl | 4-fluorophenyl |
| 4-F-5-(CF₃)-2-pyrimidinyl | 2,4-difluorophenyl |
| 4-F-5-(CF₃)-2-pyrimidinyl | 2,6-difluorophenyl |
| 4-F-5-(CF₃)-2-pyrimidinyl | 2,4,6-trifluorophenyl |

A is NHC(O)Me

| | |
|---|---|
| 5-chloro-2-pyridinyl | 2-fluorophenyl |
| 5-chloro-2-pyridinyl | 2-chlorophenyl |
| 5-chloro-2-pyridinyl | 2-bromophenyl |
| 5-chloro-2-pyridinyl | 2-methylphenyl |
| 5-chloro-2-pyridinyl | 4-fluorophenyl |
| 5-chloro-2-pyridinyl | 2,4-difluorophenyl |
| 5-chloro-2-pyridinyl | 2,6-difluorophenyl |
| 5-chloro-2-pyridinyl | 2,4,6-trifluorophenyl |
| 5-(CF₃)-2-pyridinyl | 2-fluorophenyl |
| 5-(CF₃)-2-pyridinyl | 2-chlorophenyl |
| 5-(CF₃)-2-pyridinyl | 2-bromophenyl |
| 5-(CF₃)-2-pyridinyl | 2-methylphenyl |
| 5-(CF₃)-2-pyridinyl | 4-fluorophenyl |
| 5-(CF₃)-2-pyridinyl | 2,4-difluorophenyl |
| 5-(CF₃)-2-pyridinyl | 2,6-difluorophenyl |
| 5-(CF₃)-2-pyridinyl | 2,4,6-trifluorophenyl |
| 6-chloro-3-pyridazinyl | 2-fluorophenyl |
| 6-chloro-3-pyridazinyl | 2-chlorophenyl |
| 6-chloro-3-pyridazinyl | 2-bromophenyl |
| 6-chloro-3-pyridazinyl | 2-methylphenyl |
| 6-chloro-3-pyridazinyl | 4-fluorophenyl |
| 6-chloro-3-pyridazinyl | 2,4-difluorophenyl |
| 6-chloro-3-pyridazinyl | 2,6-difluorophenyl |
| 6-chloro-3-pyridazinyl | 2,4,6-trifluorophenyl |
| 4-(CF₃)-2-pyrimidinyl | 2-fluorophenyl |
| 4-(CF₃)-2-pyrimidinyl | 2-chlorophenyl |
| 4-(CF₃)-2-pyrimidinyl | 2-bromophenyl |
| 4-(CF₃)-2-pyrimidinyl | 2-methylphenyl |
| 4-(CF₃)-2-pyrimidinyl | 4-fluorophenyl |
| 4-(CF₃)-2-pyrimidinyl | 2,4-difluorophenyl |
| 4-(CF₃)-2-pyrimidinyl | 2,6-difluorophenyl |
| 4-(CF₃)-2-pyrimidinyl | 2,4,6-trifluorophenyl |
| 4-Cl-5-F-2-pyrimidinyl | 2-fluorophenyl |
| 4-Cl-5-F-2-pyrimidinyl | 2-chlorophenyl |
| 4-Cl-5-F-2-pyrimidinyl | 2-bromophenyl |
| 4-Cl-5-F-2-pyrimidinyl | 2-methylphenyl |
| 4-Cl-5-F-2-pyrimidinyl | 4-fluorophenyl |
| 4-Cl-5-F-2-pyrimidinyl | 2,4-difluorophenyl |
| 4-Cl-5-F-2-pyrimidinyl | 2,6-difluorophenyl |
| 4-Cl-5-F-2-pyrimidinyl | 2,4,6-trifluorophenyl |
| 5-bromo-2-pyridinyl | 2-fluorophenyl |
| 5-bromo-2-pyridinyl | 2-chlorophenyl |
| 5-bromo-2-pyridinyl | 2-bromophenyl |
| 5-bromo-2-pyridinyl | 2-methylphenyl |
| 5-bromo-2-pyridinyl | 4-fluorophenyl |
| 5-bromo-2-pyridinyl | 2,4-difluorophenyl |
| 5-bromo-2-pyridinyl | 2,6-difluorophenyl |
| 5-bromo-2-pyridinyl | 2,4,6-trifluorophenyl |
| 4,5-dichloro-2-pyridinyl | 2-fluorophenyl |
| 4,5-dichloro-2-pyridinyl | 2-chlorophenyl |
| 4,5-dichloro-2-pyridinyl | 2-bromoophenyl |
| 4,5-dichloro-2-pyridinyl | 2-methylphenyl |
| 4,5-dichloro-2-pyridinyl | 4-fluorophenyl |
| 4,5-dichloro-2-pyridinyl | 2,4-difluorophenyl |
| 4,5-dichloro-2-pyridinyl | 2,6-difluorophenyl |
| 4,5-dichloro-2-pyridinyl | 2,4,6-trifluorophenyl |
| 5-(CF₃)-2-pyrimidinyl | 2-fluorophenyl |
| 5-(CF₃)-2-pyrimidinyl | 2-chlorophenyl |
| 5-(CF₃)-2-pyrimidinyl | 2-bromophenyl |
| 5-(CF₃)-2-pyrimidinyl | 2-methylphenyl |
| 5-(CF₃)-2-pyrimidinyl | 4-fluorophenyl |
| 5-(CF₃)-2-pyrimidinyl | 2,4-difluorophenyl |
| 5-(CF₃)-2-pyrimidinyl | 2,6-difluorophenyl |
| 5-(CF₃)-2-pyrimidinyl | 2,4,6-trifluorophenyl |
| 4-Cl-5-(CF₃)-2-pyrimidinyl | 2-fluorophenyl |
| 4-Cl-5-(CF₃)-2-pyrimidinyl | 2-chlorophenyl |
| 4-Cl-5-(CF₃)-2-pyrimidinyl | 2-bromophenyl |
| 4-Cl-5-(CF₃)-2-pyrimidinyl | 2-methylphenyl |
| 4-Cl-5-(CF₃)-2-pyrimidinyl | 4-fluorophenyl |
| 4-Cl-5-(CF₃)-2-pyrimidinyl | 2,4-difluorophenyl |
| 4-Cl-5-(CF₃)-2-pyrimidinyl | 2,6-difluorophenyl |
| 4-Cl-5-(CF₃)-2-pyrimidinyl | 2,4,6-trifluorophenyl |
| 4-F-5-(CF₃)-2-pyrimidinyl | 2-fluorophenyl |
| 4-F-5-(CF₃)-2-pyrimidinyl | 2-chlorophenyl |
| 4-F-5-(CF₃)-2-pyrimidinyl | 2-bromophenyl |
| 4-F-5-(CF₃)-2-pyrimidinyl | 2-methylphenyl |
| 4-F-5-(CF₃)-2-pyrimidinyl | 4-fluorophenyl |
| 4-F-5-(CF₃)-2-pyrimidinyl | 2,4-difluorophenyl |
| 4-F-5-(CF₃)-2-pyrimidinyl | 2,6-difluorophenyl |
| 4-F-5-(CF₃)-2-pyrimidinyl | 2,4,6-trifluorophenyl |

A is cyano

| | |
|---|---|
| 3-chlorophenyl | 2-pyridinyl |
| 3-chlorophenyl | 4-pyridinyl |
| 3-chlorophenyl | 3-fluoro-2-pyridinyl |
| 3-chlorophenyl | 5-fluoro-2-pyridinyl |
| 3-chlorophenyl | 3-chloro-2-pyridinyl |
| 3-chlorophenyl | 4-pyrimidinyl |
| 4-(CF₃)phenyl | 2-pyridinyl |
| 4-(CF₃)phenyl | 4-pyridinyl |
| 4-(CF₃)phenyl | 3-fluoro-2-pyridinyl |
| 4-(CF₃)phenyl | 5-fluoro-2-pyridinyl |
| 4-(CF₃)phenyl | 3-chloro-2-pyridinyl |
| 4-(CF₃)phenyl | 4-pyrimidinyl |
| 3,4-dichlorophenyl | 2-pyridinyl |
| 3,4-dichlorophenyl | 4-pyridinyl |
| 3,4-dichlorophenyl | 3-fluoro-2-pyridinyl |
| 3,4-dichlorophenyl | 5-fluoro-2-pyridinyl |
| 3,4-dichlorophenyl | 3-chloro-2-pyridinyl |
| 3,4-dichlorophenyl | 4-pyrimidinyl |
| 3-(CF₃)-4-chlorophenyl | 2-pyridinyl |
| 3-(CF₃)-4-chlorophenyl | 4-pyridinyl |
| 3-(CF₃)-4-chlorophenyl | 3-fluoro-2-pyridinyl |
| 3-(CF₃)phenyl | 2-pyridinyl |
| 3-(CF₃)phenyl | 4-pyridinyl |
| 3-(CF₃)phenyl | 3-fluoro-2-pyridinyl |
| 3-(CF₃)phenyl | 5-fluoro-2-pyridinyl |
| 3-(CF₃)phenyl | 3-chloro-2-pyridinyl |
| 3-(CF₃)phenyl | 4-pyrimidinyl |
| 4-(OCF₃)phenyl | 2-pyridinyl |
| 4-(OCF₃)phenyl | 4-pyridinyl |
| 4-(OCF₃)phenyl | 3-fluoro-2-pyridinyl |
| 4-(OCF₃)phenyl | 5-fluoro-2-pyridinyl |
| 4-(OCF₃)phenyl | 3-chloro-2-pyridinyl |
| 4-(OCF₃)phenyl | 4-pyrimidinyl |
| 3-chloro-4-(CF₃)phenyl | 2-pyridinyl |
| 3-chloro-4-(CF₃)phenyl | 4-pyridinyl |
| 3-chloro-4-(CF₃)phenyl | 3-fluoro-2-pyridinyl |
| 3-chloro-4-(CF₃)phenyl | 5-fluoro-2-pyridinyl |
| 3-chloro-4-(CF₃)phenyl | 3-chloro-2-pyridinyl |
| 3-chloro-4-(CF₃)phenyl | 4-pyrimidinyl |
| 3-(CF₃)-4-chlorophenyl | 5-fluoro-2-pyridinyl |
| 3-(CF₃)-4-chlorophenyl | 3-chloro-2-pyridinyl |
| 3-(CF₃)-4-chlorophenyl | 4-pyrimidinyl |

A is NHC(O)Me

| | |
|---|---|
| 3-chlorophenyl | 2-pyridinyl |
| 3-chlorophenyl | 4-pyridinyl |
| 3-chlorophenyl | 3-fluoro-2-pyridinyl |
| 3-chlorophenyl | 5-fluoro-2-pyridinyl |
| 3-chlorophenyl | 3-chloro-2-pyridinyl |
| 3-chlorophenyl | 4-pyrimidinyl |
| 4-(CF₃)phenyl | 2-pyridinyl |
| 4-(CF₃)phenyl | 4-pyridinyl |
| 4-(CF₃)phenyl | 3-fluoro-2-pyridinyl |
| 4-(CF₃)phenyl | 5-fluoro-2-pyridinyl |
| 4-(CF₃)phenyl | 3-chloro-2-pyridinyl |
| 4-(CF₃)phenyl | 4-pyrimidinyl |
| 3,4-dichlorophenyl | 2-pyridinyl |
| 3,4-dichlorophenyl | 4-pyridinyl |
| 3,4-dichlorophenyl | 3-fluoro-2-pyridinyl |
| 3,4-dichlorophenyl | 5-fluoro-2-pyridinyl |
| 3,4-dichlorophenyl | 3-chloro-2-pyridinyl |
| 3,4-dichlorophenyl | 4-pyrimidinyl |
| 3-(CF₃)-4-chlorophenyl | 2-pyridinyl |
| 3-(CF₃)-4-chlorophenyl | 4-pyridinyl |
| 3-(CF₃)-4-chlorophenyl | 3-fluoro-2-pyridinyl |
| 3-(CF₃)phenyl | 2-pyridinyl |
| 3-(CF₃)phenyl | 4-pyridinyl |
| 3-(CF₃)phenyl | 3-fluoro-2-pyridinyl |
| 3-(CF₃)phenyl | 5-fluoro-2-pyridinyl |
| 3-(CF₃)phenyl | 3-chloro-2-pyridinyl |
| 3-(CF₃)phenyl | 4-pyrimidinyl |
| 4-(OCF₃)phenyl | 2-pyridinyl |
| 4-(OCF₃)phenyl | 4-pyridinyl |
| 4-(OCF₃)phenyl | 3-fluoro-2-pyridinyl |
| 4-(OCF₃)phenyl | 5-fluoro-2-pyridinyl |
| 4-(OCF₃)phenyl | 3-chloro-2-pyridinyl |
| 4-(OCF₃)phenyl | 4-pyrimidinyl |
| 3-chloro-4-(CF₃)phenyl | 2-pyridinyl |
| 3-chloro-4-(CF₃)phenyl | 4-pyridinyl |
| 3-chloro-4-(CF₃)phenyl | 3-fluoro-2-pyridinyl |
| 3-chloro-4-(CF₃)phenyl | 5-fluoro-2-pyridinyl |
| 3-chloro-4-(CF₃)phenyl | 3-chloro-2-pyridinyl |
| 3-chloro-4-(CF₃)phenyl | 4-pyrimidinyl |
| 3-(CF₃)-4-chlorophenyl | 5-fluoro-2-pyridinyl |
| 3-(CF₃)-4-chlorophenyl | 3-chloro-2-pyridinyl |
| 3-(CF₃)-4-chlorophenyl | 4-pyrimidinyl |

TABLE 4

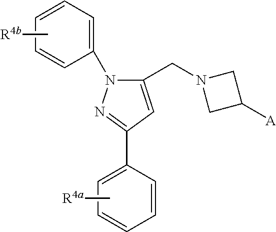

| | |
|---|---|
| R^{4b} | R^{4a} |

A is cyano

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

A is NHC(O)Me

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

TABLE 5

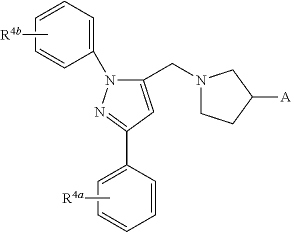

| | |
|---|---|
| R^{4b} | R^{4a} |

A is cyano

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

A is NH(O)Me

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

TABLE 6

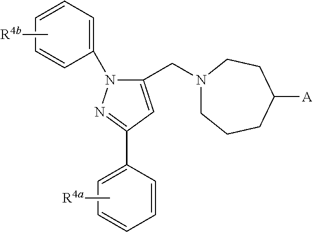

| | |
|---|---|
| R^{4b} | R^{4a} |

A is cyano

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

A is NHC(O)Me

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

TABLE 7

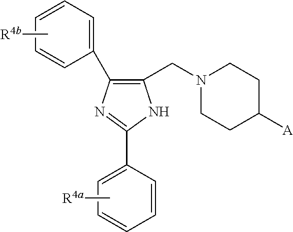

| | |
|---|---|
| R^{4b} | R^{4a} |

A is cyano

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is CO₂Me

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is CO₂Et

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 9.-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is NHC(O)Me

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is C(O)Me

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is NHC(O)OMe

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 3-OCF₃ |
| 2-Cl | 3-OCF₃ |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is 1,3,4-oxadiazole

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-OCF₃ |
| 2-F, 4-F, 6-F | 4-OCF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

TABLE 8

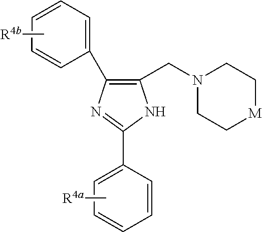

| | |
|---|---|
| R^{4b} | R^{4a} |

M is O

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is S

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is NMe

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(phenyl)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(Me)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(CF₃)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(OC(O)Me)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(C≡CH)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(CH═CH₂)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(4-pyridinyl)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(2-imidazolyl)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(3-(1,2,4-triazolyl)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(2-(1,3,4-oxadiazolyl)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

TABLE 9

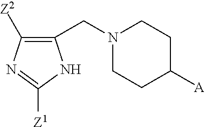

| | |
|---|---|
| Z¹ | Z² |

A is cyano

| | |
|---|---|
| 5-chloro-2-pyridinyl | 2-fluorophenyl |
| 5-chloro-2-pyridinyl | 2-chlorophenyl |
| 5-chloro-2-pyridinyl | 2-bromophenyl |
| 5-chloro-2-pyridinyl | 2-methylphenyl |
| 5-chloro-2-pyridinyl | 4-fluorophenyl |
| 5-chloro-2-pyridinyl | 2,4-difluorophenyl |
| 5-chloro-2-pyridinyl | 2,6-difluorophenyl |
| 5-chloro-2-pyridinyl | 2,4,6-trifluorophenyl |
| 5-(CF₃)-2-pyridinyl | 2-fluorophenyl |
| 5-(CF₃)-2-pyridinyl | 2-chlorophenyl |
| 5-(CF₃)-2-pyridinyl | 2-bromophenyl |
| 5-(CF₃)-2-pyridinyl | 2-methylphenyl |
| 5-(CF₃)-2-pyridinyl | 4-fluorophenyl |
| 5-(CF₃)-2-pyridinyl | 2,4-difluorophenyl |
| 5-(CF₃)-2-pyridinyl | 2,6-difluorophenyl |
| 5-(CF₃)-2-pyridinyl | 2,4,6-trifluorophenyl |
| 6-chloro-3-pyridazinyl | 2-fluorophenyl |
| 6-chloro-3-pyridazinyl | 2-chlorophenyl |
| 6-chloro-3-pyridazinyl | 2-bromophenyl |
| 6-chloro-3-pyridazinyl | 2-methylphenyl |
| 6-chloro-3-pyridazinyl | 4-fluorophenyl |
| 6-chloro-3-pyridazinyl | 2,4-difluorophenyl |
| 6-chloro-3-pyridazinyl | 2,6-difluorophenyl |
| 6-chloro-3-pyridazinyl | 2,4,6-trifluorophenyl |
| 4-(CF₃)-2-pyrimidinyl | 2-fluorophenyl |
| 4-(CF₃)-2-pyrimidinyl | 2-chlorophenyl |
| 4-(CF₃)-2-pyrimidinyl | 2-bromophenyl |
| 4-(CF₃)-2-pyrimidinyl | 2-methylphenyl |
| 4-(CF₃)-2-pyrimidinyl | 4-fluorophenyl |
| 4-(CF₃)-2-pyrimidinyl | 2,4-difluorophenyl |
| 4-(CF₃)-2-pyrimidinyl | 2,6-difluorophenyl |
| 4-(CF₃)-2-pyrimidinyl | 2,4,6-trifluorophenyl |
| 4-Cl-5-F-2-pyrimidinyl | 2-fluorophenyl |
| 4-Cl-5-F-2-pyrimidinyl | 2-chlorophenyl |
| 4-Cl-5-F-2-pyrimidinyl | 2-bromophenyl |
| 4-Cl-5-F-2-pyrimidinyl | 2-methylphenyl |
| 4-Cl-5-F-2-pyrimidinyl | 4-fluorophenyl |
| 4-Cl-5-F-2-pyrimidinyl | 2,4-difluorophenyl |
| 4-Cl-5-F-2-pyrimidinyl | 2,6-difluorophenyl |
| 4-Cl-5-F-2-pyrimidinyl | 2,4,6-trifluorophenyl |
| 5-bromo-2-pyridinyl | 2-fluorophenyl |
| 5-bromo-2-pyridinyl | 2-chlorophenyl |
| 5-bromo-2-pyridinyl | 2-bromophenyl |
| 5-bromo-2-pyridinyl | 2-methylphenyl |
| 5-bromo-2-pyridinyl | 4-fluorophenyl |
| 5-bromo-2-pyridinyl | 2,4-difluorophenyl |
| 5-bromo-2-pyridinyl | 2,6-difluorophenyl |
| 5-bromo-2-pyridinyl | 2,4,6-trifluorophenyl |
| 4,5-dichloro-2-pyridinyl | 2-fluorophenyl |
| 4,5-dichloro-2-pyridinyl | 2-chlorophenyl |
| 4,5-dichloro-2-pyridinyl | 2-bromophenyl |
| 4,5-dichloro-2-pyridinyl | 2-methylphenyl |
| 4,5-dichloro-2-pyridinyl | 4-fluorophenyl |
| 4,5-dichloro-2-pyridinyl | 2,4-difluorophenyl |
| 4,5-dichloro-2-pyridinyl | 2,6-difluorophenyl |
| 4,5-dichloro-2-pyridinyl | 2,4,6-trifluorophenyl |
| 5-(CF₃)-2-pyrimidinyl | 2-fluorophenyl |
| 5-(CF₃)-2-pyrimidinyl | 2-chlorophenyl |
| 5-(CF₃)-2-pyrimidinyl | 2-bromophenyl |
| 5-(CF₃)-2-pyrimidinyl | 2-methylphenyl |
| 5-(CF₃)-2-pyrimidinyl | 4-fluorophenyl |
| 5-(CF₃)-2-pyrimidinyl | 2,4-difluorophenyl |
| 5-(CF₃)-2-pyrimidinyl | 2,6-difluorophenyl |
| 5-(CF₃)-2-pyrimidinyl | 2,4,6-trifluorophenyl |
| 4-Cl-5-(CF₃)-2-pyrimidinyl | 2-fluorophenyl |
| 4-Cl-5-(CF₃)-2-pyrimidinyl | 2-chlorophenyl |
| 4-Cl-5-(CF₃)-2-pyrimidinyl | 2-bromophenyl |
| 4-Cl-5-(CF₃)-2-pyrimidinyl | 2-methylphenyl |
| 4-Cl-5-(CF₃)-2-pyrimidinyl | 4-fluorophenyl |
| 4-Cl-5-(CF₃)-2-pyrimidinyl | 2,4-difluorophenyl |
| 4-Cl-5-(CF₃)-2-pyrimidinyl | 2,6-difluorophenyl |
| 4-Cl-5-(CF₃)-2-pyrimidinyl | 2,4,6-trifluorophenyl |
| 4-Cl-5-(CF₃)-2-pyrimidinyl | 2-fluorophenyl |
| 4-F-5-(CF₃)-2-pyrimidinyl | 2-chlorophenyl |
| 4-F-5-(CF₃)-2-pyrimidinyl | 2-bromophenyl |
| 4-F-5-(CF₃)-2-pyrimidinyl | 2-methylphenyl |
| 4-F-5-(CF₃)-2-pyrimidinyl | 4-fluorophenyl |
| 4-F-5-(CF₃)-2-pyrimidinyl | 2,4-difluorophenyl |
| 4-F-5-(CF₃)-2-pyrimidinyl | 2,6-difluorophenyl |
| 4-F-5-(CF₃)-2-pyrimidinyl | 2,4,6-trifluorophenyl |

A is NHC(O)Me

| | |
|---|---|
| 5-chloro-2-pyridinyl | 2-fluorophenyl |
| 5-chloro-2-pyridinyl | 2-chlorophenyl |
| 5-chloro-2-pyridinyl | 2-bromophenyl |
| 5-chloro-2-pyridinyl | 2-methylphenyl |
| 5-chloro-2-pyridinyl | 4-fluorophenyl |
| 5-chloro-2-pyridinyl | 2,4-difluorophenyl |
| 5-chloro-2-pyridinyl | 2,6-difluorophenyl |
| 5-chloro-2-pyridinyl | 2,4,6-trifluorophenyl |
| 5-(CF₃)-2-pyridinyl | 2-fluorophenyl |
| 5-(CF₃)-2-pyridinyl | 2-chlorophenyl |
| 5-(CF₃)-2-pyridinyl | 2-bromophenyl |
| 5-(CF₃)-2-pyridinyl | 2-methylphenyl |
| 5-(CF₃)-2-pyridinyl | 4-fluorophenyl |
| 5-(CF₃)-2-pyridinyl | 2,4-difluorophenyl |
| 5-(CF₃)-2-pyridinyl | 2,6-difluorophenyl |
| 5-(CF₃)-2-pyridinyl | 2,4,6-trifluorophenyl |
| 6-chloro-3-pyridazinyl | 2-fluorophenyl |
| 6-chloro-3-pyridazinyl | 2-chlorophenyl |
| 6-chloro-3-pyridazinyl | 2-bromophenyl |
| 6-chloro-3-pyridazinyl | 2-methylphenyl |
| 6-chloro-3-pyridazinyl | 4-fluorophenyl |
| 6-chloro-3-pyridazinyl | 2,4-difluorophenyl |
| 6-chloro-3-pyridazinyl | 2,6-difluorophenyl |
| 6-chloro-3-pyridazinyl | 2,4,6-trifluorophenyl |
| 4-(CF₃)-2-pyrimidinyl | 2-fluorophenyl |
| 4-(CF₃)-2-pyrimidinyl | 2-chlorophenyl |
| 4-(CF₃)-2-pyrimidinyl | 2-bromophenyl |
| 4-(CF₃)-2-pyrimidinyl | 2-methylphenyl |
| 4-(CF₃)-2-pyrimidinyl | 4-fluorophenyl |
| 4-(CF₃)-2-pyrimidinyl | 2,4-difluorophenyl |
| 4-(CF₃)-2-pyrimidinyl | 2,6-difluorophenyl |
| 4-(CF₃)-2-pyrimidinyl | 2,4,6-trifluorophenyl |
| 4-Cl-5-F-2-pyrimidinyl | 2-fluorophenyl |
| 4-Cl-5-F-2-pyrimidinyl | 2-chlorophenyl |
| 4-Cl-5-F-2-pyrimidinyl | 2-bromophenyl |
| 4-Cl-5-F-2-pyrimidinyl | 2-methylphenyl |
| 4-Cl-5-F-2-pyrimidinyl | 4-fluorophenyl |
| 4-Cl-5-F-2-pyrimidinyl | 2,4-difluorophenyl |
| 4-Cl-5-F-2-pyrimidinyl | 2,6-difluorophenyl |
| 4-Cl-5-F-2-pyrimidinyl | 2,4,6-trifluorophenyl |
| 5-bromo-2-pyridinyl | 2-fluorophenyl |
| 5-bromo-2-pyridinyl | 2-chlorophenyl |
| 5-bromo-2-pyridinyl | 2-bromophenyl |
| 5-bromo-2-pyridinyl | 2-methylphenyl |
| 5-bromo-2-pyridinyl | 4-fluorophenyl |
| 5-bromo-2-pyridinyl | 2,4-difluorophenyl |
| 5-bromo-2-pyridinyl | 2,6-difluorophenyl |
| 5-bromo-2-pyridinyl | 2,4,6-trifluorophenyl |
| 4,5-dichloro-2-pyridinyl | 2-fluorophenyl |
| 4,5-dichloro-2-pyridinyl | 2-chlorophenyl |
| 4,5-dichloro-2-pyridinyl | 2-bromophenyl |
| 4,5-dichloro-2-pyridinyl | 2-methylphenyl |
| 4,5-dichloro-2-pyridinyl | 4-fluorophenyl |
| 4,5-dichloro-2-pyridinyl | 2,4-difluorophenyl |
| 4,5-dichloro-2-pyridinyl | 2,6-difluorophenyl |
| 4,5-dichloro-2-pyridinyl | 2,4,6-trifluorophenyl |
| 5-(CF₃)-2-pyrimidinyl | 2-fluorophenyl |
| 5-(CF₃)-2-pyrimidinyl | 2-chlorophenyl |
| 5-(CF₃)-2-pyrimidinyl | 2-bromophenyl |
| 5-(CF₃)-2-pyrimidinyl | 2-methylphenyl |
| 5-(CF₃)-2-pyrimidinyl | 4-fluorophenyl |
| 5-(CF₃)-2-pyrimidinyl | 2,4-difluorophenyl |
| 5-(CF₃)-2-pyrimidinyl | 2,6-difluorophenyl |
| 5-(CF₃)-2-pyrimidinyl | 2,4,6-trifluorophenyl |
| 4-Cl-5-(CF₃)-2-pyrimidinyl | 2-fluorophenyl |
| 4-Cl-5-(CF₃)-2-pyrimidinyl | 2-chlorophenyl |
| 4-Cl-5-(CF₃)-2-pyrimidinyl | 2-bromophenyl |
| 4-Cl-5-(CF₃)-2-pyrimidinyl | 2-methylphenyl |
| 4-Cl-5-(CF₃)-2-pyrimidinyl | 4-fluorophenyl |
| 4-Cl-5-(CF₃)-2-pyrimidinyl | 2,4-difluorophenyl |
| 4-Cl-5-(CF₃)-2-pyrimidinyl | 2,6-difluorophenyl |
| 4-Cl-5-(CF₃)-2-pyrimidinyl | 2,4,6-trifluorophenyl |
| 4-F-5-(CF₃)-2-pyrimidinyl | 2-fluorophenyl |
| 4-F-5-(CF₃)-2-pyrimidinyl | 2-chlorophenyl |
| 4-F-5-(CF₃)-2-pyrimidinyl | 2-bromophenyl |
| 4-F-5-(CF₃)-2-pyrimidinyl | 2-methylphenyl |
| 4-F-5-(CF₃)-2-pyrimidinyl | 4-fluorophenyl |
| 4-F-5-(CF₃)-2-pyrimidinyl | 2,4-difluorophenyl |
| 4-F-5-(CF₃)-2-pyrimidinyl | 2,6-difluorophenyl |
| 4-F-5-(CF₃)-2-pyrimidinyl | 2,4,6-trifluorophenyl |

TABLE 10

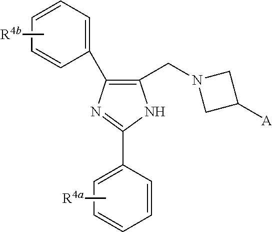

| | |
|---|---|
| R^{4b} | R^{4a} |

A is cyano

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

A is NHC(O)Me

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

TABLE 11

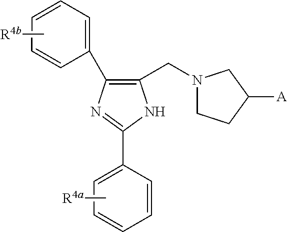

| | |
|---|---|
| R^{4b} | R^{4a} |

A is cyano

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

A is NHC(O)Me

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

TABLE 12

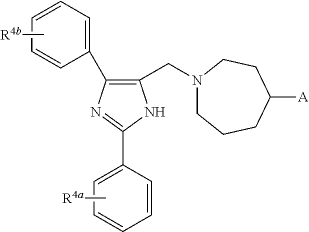

| | |
|---|---|
| R^{4b} | R^{4a} |

A is cyano

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

A is NHC(O)Me

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

TABLE 13

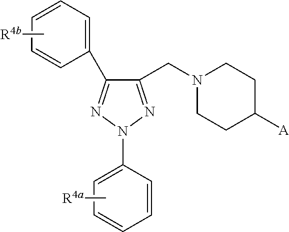

| | |
|---|---|
| R^{4b} | R^{4a} |

A is cyano

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is CO₂Me

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is CO₂Et

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is NHC(O)Me

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is C(O)Me

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is NHC(O)OMe

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is 1,3,4-oxadiazole

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-OCF₃ |
| 2-F, 4-F, 6-F | 3-F, 4-OCF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

TABLE 14

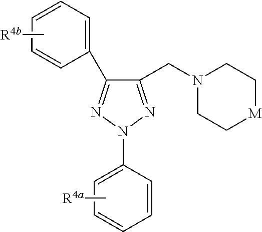

| | |
|---|---|
| R^{4b} | R^{4a} |

M is O

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is S

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is NMe

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(phenyl)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(Me)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(CF₃)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(OC(O)Me)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(C≡CH)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(CH═CH₂)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(4-pyridinyl)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(2-imidazolyl)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

M is CH(3-(1,2,4-triazolyl))

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |

M is CH(2-(1,3,4-oxadiazolyl)

| | |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

TABLE 15

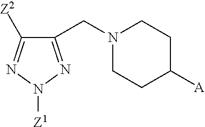

| | |
|---|---|
| Z¹ | Z² |

A is cyano

| | |
|---|---|
| 5-chloro-2-pyridinyl | 2-fluorophenyl |
| 5-chloro-2-pyridinyl | 2-chlorophenyl |
| 5-chloro-2-pyridinyl | 2-bromophenyl |
| 5-chloro-2-pyridinyl | 2-methylphenyl |
| 5-chloro-2-pyridinyl | 4-fluorophenyl |
| 5-chloro-2-pyridinyl | 2,4-difluorophenyl |
| 5-chloro-2-pyridinyl | 2,6-difluorophenyl |
| 5-chloro-2-pyridinyl | 2,4,6-trifluorophenyl |
| 5-(CF₃)-2-pyridinyl | 2-fluorophenyl |
| 5-(CF₃)-2-pyridinyl | 2-chlorophenyl |
| 5-(CF₃)-2-pyridinyl | 2-bromophenyl |
| 5-(CF₃)-2-pyridinyl | 2-methylphenyl |
| 5-(CF₃)-2-pyridinyl | 4-fluorophenyl |
| 5-(CF₃)-2-pyridinyl | 2,4-difluorophenyl |
| 5-(CF₃)-2-pyridinyl | 2,6-difluorophenyl |
| 5-(CF₃)-2-pyridinyl | 2,4,6-trifluorophenyl |
| 6-chloro-3-pyridazinyl | 2-fluorophenyl |
| 6-chloro-3-pyridazinyl | 2-chlorophenyl |
| 6-chloro-3-pyridazinyl | 2-bromophenyl |
| 6-chloro-3-pyridazinyl | 2-methylphenyl |
| 6-chloro-3-pyridazinyl | 4-fluorophenyl |
| 6-chloro-3-pyridazinyl | 2,4-difluorophenyl |
| 6-chloro-3-pyridazinyl | 2,6-difluorophenyl |
| 6-chloro-3-pyridazinyl | 2,4,6-trifluorophenyl |
| 4-(CF₃)-2-pyrimidinyl | 2-fluorophenyl |
| 4-(CF₃)-2-pyrimidinyl | 2-chlorophenyl |
| 4-(CF₃)-2-pyrimidinyl | 2-bromophenyl |
| 4-(CF₃)-2-pyrimidinyl | 2-methylphenyl |
| 4-(CF₃)-2-pyrimidinyl | 4-fluorophenyl |
| 4-(CF₃)-2-pyrimidinyl | 2,4-difluorophenyl |
| 4-(CF₃)-2-pyrimidinyl | 2,6-difluorophenyl |
| 4-(CF₃)-2-pyrimidinyl | 2,4,6-trifluorophenyl |
| 4-Cl-5-F-2-pyrimidinyl | 2-fluorophenyl |
| 4-Cl-5-F-2-pyrimidinyl | 2-chlorophenyl |
| 4-Cl-5-F-2-pyrimidinyl | 2-bromophenyl |
| 4-Cl-5-F-2-pyrimidinyl | 2-methylphenyl |
| 4-Cl-5-F-2-pyrimidinyl | 4-fluorophenyl |
| 4-Cl-5-F-2-pyrimidinyl | 2,4-difluorophenyl |
| 4-Cl-5-F-2-pyrimidinyl | 2,6-difluorophenyl |
| 4-Cl-5-F-2-pyrimidinyl | 2,4,6-trifluorophenyl |
| 5-bromo-2-pyridinyl | 2-fluorophenyl |
| 5-bromo-2-pyridinyl | 2-chlorophenyl |
| 5-bromo-2-pyridinyl | 2-bromophenyl |
| 5-bromo-2-pyridinyl | 2-methylphenyl |
| 5-bromo-2-pyridinyl | 4-fluorophenyl |
| 5-bromo-2-pyridinyl | 2,4-difluorophenyl |
| 5-bromo-2-pyridinyl | 2,6-difluorophenyl |
| 5-bromo-2-pyridinyl | 2,4,6-trifluorophenyl |
| 4,5-dichloro-2-pyridinyl | 2-fluorophenyl |
| 4,5-dichloro-2-pyridinyl | 2-chlorophenyl |
| 4,5-dichloro-2-pyridinyl | 2-bromophenyl |
| 4,5-dichloro-2-pyridinyl | 2-methylphenyl |
| 4,5-dichloro-2-pyridinyl | 4-fluorophenyl |
| 4,5-dichloro-2-pyridinyl | 2,4-difluorophenyl |
| 4,5-dichloro-2-pyridinyl | 2,6-difluorophenyl |
| 4,5-dichloro-2-pyridinyl | 2,4,6-trifluorophenyl |
| 5-(CF₃)-2-pyrimidinyl | 2-fluorophenyl |
| 5-(CF₃)-2-pyrimidinyl | 2-chlorophenyl |
| 5-(CF₃)-2-pyrimidinyl | 2-bromophenyl |
| 5-(CF₃)-2-pyrimidinyl | 2-methylphenyl |
| 5-(CF₃)-2-pyrimidinyl | 4-fluorophenyl |
| 5-(CF₃)-2-pyrimidinyl | 2,4-difluorophenyl |
| 5-(CF₃)-2-pyrimidinyl | 2,6-difluorophenyl |
| 5-(CF₃)-2-pyrimidinyl | 2,4,6-trifluorophenyl |
| 4-Cl-5-(CF₃)-2-pyrimidinyl | 2-fluorophenyl |
| 4-Cl-5-(CF₃)-2-pyrimidinyl | 2-chlorophenyl |
| 4-Cl-5-(CF₃)-2-pyrimidinyl | 2-bromophenyl |
| 4-Cl-5-(CF₃)-2-pyrimidinyl | 2-methylphenyl |
| 4-Cl-5-(CF₃)-2-pyrimidinyl | 4-fluorophenyl |
| 4-Cl-5-(CF₃)-2-pyrimidinyl | 2,4-difluorophenyl |
| 4-Cl-5-(CF₃)-2-pyrimidinyl | 2,6-difluorophenyl |
| 4-Cl-5-(CF₃)-2-pyrimidinyl | 2,4,6-trifluorophenyl |
| 4-F-5-(CF₃)-2-pyrimidinyl | 2-fluorophenyl |
| 4-F-5-(CF₃)-2-pyrimidinyl | 2-chlorophenyl |
| 4-F-5-(CF₃)-2-pyrimidinyl | 2-bromophenyl |
| 4-F-5-(CF₃)-2-pyrimidinyl | 2-methylphenyl |
| 4-F-5-(CF₃)-2-pyrimidinyl | 4-fluorophenyl |
| 4-F-5-(CF₃)-2-pyrimidinyl | 2,4-difluorophenyl |
| 4-F-5-(CF₃)-2-pyrimidinyl | 2,6-difluorophenyl |
| 4-F-5-(CF₃)-2-pyrimidinyl | 2,4,6-trifluorophenyl |

A is NHC(O)Me

| | |
|---|---|
| 5-chloro-2-pyridinyl | 2-fluorophenyl |
| 5-chloro-2-pyridinyl | 2-chlorophenyl |
| 5-chloro-2-pyridinyl | 2-bromophenyl |
| 5-chloro-2-pyridinyl | 2-methylphenyl |
| 5-chloro-2-pyridinyl | 4-fluorophenyl |
| 5-chloro-2-pyridinyl | 2,4-difluorophenyl |
| 5-chloro-2-pyridinyl | 2,6-difluorophenyl |
| 5-chloro-2-pyridinyl | 2,4,6-trifluorophenyl |
| 5-(CF₃)-2-pyridinyl | 2-fluorophenyl |
| 5-(CF₃)-2-pyridinyl | 2-chlorophenyl |
| 5-(CF₃)-2-pyridinyl | 2-bromophenyl |
| 5-(CF₃)-2-pyridinyl | 2-methylphenyl |
| 5-(CF₃)-2-pyridinyl | 4-fluorophenyl |
| 5-(CF₃)-2-pyridinyl | 2,4-difluorophenyl |
| 5-(CF₃)-2-pyridinyl | 2,6-difluorophenyl |
| 5-(CF₃)-2-pyridinyl | 2,4,6-trifluorophenyl |
| 6-chloro-3-pyridazinyl | 2-fluorophenyl |
| 6-chloro-3-pyridazinyl | 2-chlorophenyl |
| 6-chloro-3-pyridazinyl | 2-bromophenyl |
| 6-chloro-3-pyridazinyl | 2-methylphenyl |
| 6-chloro-3-pyridazinyl | 4-fluorophenyl |
| 6-chloro-3-pyridazinyl | 2,4-difluorophenyl |
| 6-chloro-3-pyridazinyl | 2,6-difluorophenyl |
| 6-chloro-3-pyridazinyl | 2,4,6-trifluorophenyl |
| 4-(CF₃)-2-pyrimidinyl | 2-fluorophenyl |
| 4-(CF₃)-2-pyrimidinyl | 2-chlorophenyl |
| 4-(CF₃)-2-pyrimidinyl | 2-bromophenyl |
| 4-(CF₃)-2-pyrimidinyl | 2-methylphenyl |
| 4-(CF₃)-2-pyrimidinyl | 4-fluorophenyl |
| 4-(CF₃)-2-pyrimidinyl | 2,4-difluorophenyl |
| 4-(CF₃)-2-pyrimidinyl | 2,6-difluorophenyl |
| 4-(CF₃)-2-pyrimidinyl | 2,4,6-trifluorophenyl |
| 4-Cl-5-F-2-pyrimidinyl | 2-fluorophenyl |
| 4-Cl-5-F-2-pyrimidinyl | 2-chlorophenyl |
| 4-Cl-5-F-2-pyrimidinyl | 2-bromophenyl |
| 4-Cl-5-F-2-pyrimidinyl | 2-methylphenyl |
| 4-Cl-5-F-2-pyrimidinyl | 4-fluorophenyl |
| 4-Cl-5-F-2-pyrimidinyl | 2,4-difluorophenyl |
| 4-Cl-5-F-2-pyrimidinyl | 2,6-difluorophenyl |
| 4-Cl-5-F-2-pyrimidinyl | 2,4,6-trifluorophenyl |
| 5-bromo-2-pyridinyl | 2-fluorophenyl |
| 5-bromo-2-pyridinyl | 2-chlorophenyl |
| 5-bromo-2-pyridinyl | 2-bromophenyl |
| 5-bromo-2-pyridinyl | 2-methylphenyl |
| 5-bromo-2-pyridinyl | 4-fluorophenyl |
| 5-bromo-2-pyridinyl | 2,4-difluorophenyl |
| 5-bromo-2-pyridinyl | 2,6-difluorophenyl |
| 5-bromo-2-pyridinyl | 2,4,6-trifluorophenyl |
| 4,5-dichloro-2-pyridinyl | 2-fluorophenyl |
| 4,5-dichloro-2-pyridinyl | 2-chlorophenyl |
| 4,5-dichloro-2-pyridinyl | 2-bromophenyl |
| 4,5-dichloro-2-pyridinyl | 2-methylphenyl |
| 4,5-dichloro-2-pyridinyl | 4-fluorophenyl |
| 4,5-dichloro-2-pyridinyl | 2,4-difluorophenyl |
| 4,5-dichloro-2-pyridinyl | 2,6-difluorophenyl |
| 4,5-dichloro-2-pyridinyl | 2,4,6-trifluorophenyl |
| 5-(CF₃)-2-pyrimidinyl | 2-fluorophenyl |
| 5-(CF₃)-2-pyrimidinyl | 2-chlorophenyl |
| 5-(CF₃)-2-pyrimidinyl | 2-bromophenyl |
| 5-(CF₃)-2-pyrimidinyl | 2-methylphenyl |
| 5-(CF₃)-2-pyrimidinyl | 4-fluorophenyl |
| 5-(CF₃)-2-pyrimidinyl | 2,4-difluorophenyl |
| 5-(CF₃)-2-pyrimidinyl | 2,6-difluorophenyl |
| 5-(CF₃)-2-pyrimidinyl | 2,4,6-trifluorophenyl |
| 4-Cl-5-(CF₃)-2-pyrimidinyl | 2-fluorophenyl |
| 4-Cl-5-(CF₃)-2-pyrimidinyl | 2-chlorophenyl |
| 4-Cl-5-(CF₃)-2-pyrimidinyl | 2-bromophenyl |
| 4-Cl-5-(CF₃)-2-pyrimidinyl | 2-methylphenyl |
| 4-Cl-5-(CF₃)-2-pyrimidinyl | 4-fluorophenyl |
| 4-Cl-5-(CF₃)-2-pyrimidinyl | 2,4-difluorophenyl |
| 4-Cl-5-(CF₃)-2-pyrimidinyl | 2,6-difluorophenyl |
| 4-Cl-5-(CF₃)-2-pyrimidinyl | 2,4,6-trifluorophenyl |
| 4-F-5-(CF₃)-2-pyrimidinyl | 2-fluorophenyl |
| 4-F-5-(CF₃)-2-pyrimidinyl | 2-chlorophenyl |
| 4-F-5-(CF₃)-2-pyrimidinyl | 2-bromophenyl |
| 4-F-5-(CF₃)-2-pyrimidinyl | 2-methylphenyl |
| 4-F-5-(CF₃)-2-pyrimidinyl | 4-fluorophenyl |
| 4-F-5-(CF₃)-2-pyrimidinyl | 2,4-difluorophenyl |
| 4-F-5-(CF₃)-2-pyrimidinyl | 2,6-difluorophenyl |
| 4-F-5-(CF₃)-2-pyrimidinyl | 2,4,6-trifluorophenyl |

A is cyano

| | |
|---|---|
| 3-chlorophenyl | 2-pyridinyl |
| 3-chlorophenyl | 4-pyridinyl |
| 3-chlorophenyl | 3-fluoro-2-pyridinyl |
| 3-chlorophenyl | 5-fluoro-2-pyridinyl |
| 3-chlorophenyl | 3-chloro-2-pyridinyl |
| 3-chlorophenyl | 4-pyrimidinyl |
| 4-(CF₃)phenyl | 2-pyridinyl |
| 4-(CF₃)phenyl | 4-pyridinyl |
| 4-(CF₃)phenyl | 3-fluoro-2-pyridinyl |
| 4-(CF₃)phenyl | 5-fluoro-2-pyridinyl |
| 4-(CF₃)phenyl | 3-chloro-2-pyridinyl |
| 4-(CF₃)phenyl | 4-pyrimidinyl |
| 3,4-dichlorophenyl | 2-pyridinyl |
| 3,4-dichlorophenyl | 4-pyridinyl |
| 3,4-dichlorophenyl | 3-fluoro-2-pyridinyl |
| 3,4-dichlorophenyl | 5-fluoro-2-pyridinyl |
| 3,4-dichlorophenyl | 3-chloro-2-pyridinyl |
| 3,4-dichlorophenyl | 4-pyrimidinyl |
| 3-(CF₃)-4-chlorophenyl | 2-pyridinyl |
| 3-(CF₃)-4-chlorophenyl | 4-pyridinyl |
| 3-(CF₃)-4-chlorophenyl | 3-fluoro-2-pyridinyl |
| 3-(CF₃)phenyl | 2-pyridinyl |
| 3-(CF₃)phenyl | 4-pyridinyl |
| 3-(CF₃)phenyl | 3-fluoro-2-pyridinyl |
| 3-(CF₃)phenyl | 5-fluoro-2-pyridinyl |
| 3-(CF₃)phenyl | 3-chloro-2-pyridinyl |
| 3-(CF₃)phenyl | 4-pyrimidinyl |
| 4-(OCF₃)phenyl | 2-pyridinyl |
| 4-(OCF₃)phenyl | 4-pyridinyl |
| 4-(OCF₃)phenyl | 3-fluoro-2-pyridinyl |
| 4-(OCF₃)phenyl | 5-fluoro-2-pyridinyl |
| 4-(OCF₃)phenyl | 3-chloro-2-pyridinyl |
| 4-(OCF₃)phenyl | 4-pyrimidinyl |
| 3-chloro-4-(CF₃)phenyl | 2-pyridinyl |
| 3-chloro-4-(CF₃)phenyl | 4-pyridinyl |
| 3-chloro-4-(CF₃)phenyl | 3-fluoro-2-pyridinyl |
| 3-chloro-4-(CF₃)phenyl | 5-fluoro-2-pyridinyl |
| 3-chloro-4-(CF₃)phenyl | 3-chloro-2-pyridinyl |
| 3-chloro-4-(CF₃)phenyl | 4-pyrimidinyl |
| 3-(CF₃)-4-chlorophenyl | 5-fluoro-2-pyridinyl |
| 3-(CF₃)-4-chlorophenyl | 3-chloro-2-pyridinyl |
| 3-(CF₃)-4-chlorophenyl | 4-pyrimidinyl |

A is NHC(O)Me

| | |
|---|---|
| 3-chlorophenyl | 2-pyridinyl |
| 3-chlorophenyl | 4-pyridinyl |
| 3-chlorophenyl | 3-fluoro-2-pyridinyl |
| 3-chlorophenyl | 5-fluoro-2-pyridinyl |
| 3-chlorophenyl | 3-chloro-2-pyridinyl |
| 3-chlorophenyl | 4-pyrimidinyl |
| 4-(CF₃)phenyl | 2-pyridinyl |
| 4-(CF₃)phenyl | 4-pyridinyl |
| 4-(CF₃)phenyl | 3-fluoro-2-pyridinyl |
| 4-(CF₃)phenyl | 5-fluoro-2-pyridinyl |
| 4-(CF₃)phenyl | 3-chloro-2-pyridinyl |
| 4-(CF₃)phenyl | 4-pyrimidinyl |
| 3,4-dichlorophenyl | 2-pyridinyl |
| 3,4-dichlorophenyl | 4-pyridinyl |
| 3,4-dichlorophenyl | 3-fluoro-2-pyridinyl |
| 3,4-dichlorophenyl | 5-fluoro-2-pyridinyl |
| 3,4-dichlorophenyl | 3-chloro-2-pyridinyl |
| 3,4-dichlorophenyl | 4-pyrimidinyl |
| 3-(CF₃)-4-chlorophenyl | 2-pyridinyl |
| 3-(CF₃)-4-chlorophenyl | 4-pyridinyl |
| 3-(CF₃)-4-chlorophenyl | 3-fluoro-2-pyridinyl |
| 3-(CF₃)phenyl | 2-pyridinyl |
| 3-(CF₃)phenyl | 4-pyridinyl |
| 3-(CF₃)phenyl | 3-fluoro-2-pyridinyl |
| 3-(CF₃)phenyl | 5-fluoro-2-pyridinyl |
| 3-(CF₃)phenyl | 3-chloro-2-pyridinyl |
| 3-(CF₃)phenyl | 4-pyrimidinyl |
| 4-(OCF₃)phenyl | 2-pyridinyl |
| 4-(OCF₃)phenyl | 4-pyridinyl |
| 4-(OCF₃)phenyl | 3-fluoro-2-pyridinyl |
| 4-(OCF₃)phenyl | 5-fluoro-2-pyridinyl |
| 4-(OCF₃)phenyl | 3-chloro-2-pyridinyl |
| 4-(OCF₃)phenyl | 4-pyrimidinyl |
| 3,4-dichlorophenyl | 2-pyridinyl |
| 3,4-dichlorophenyl | 4-pyridinyl |
| 3,4-dichlorophenyl | 3-fluoro-2-pyridinyl |
| 3,4-dichlorophenyl | 5-fluoro-2-pyridinyl |
| 3,4-dichlorophenyl | 3-chloro-2-pyridinyl |
| 3,4-dichlorophenyl | 4-pyrimidinyl |
| 3-(CF₃)-4-chlorophenyl | 5-fluoro-2-pyridinyl |
| 3-(CF₃)-4-chlorophenyl | 3-chloro-2-pyridinyl |
| 3-(CF₃)-4-chlorophenyl | 4-pyrimidinyl |

TABLE 16

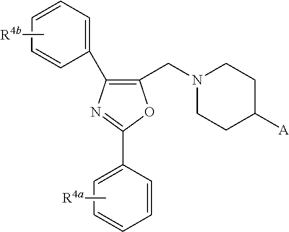

| | |
|---|---|
| R^{4b} | R^{4a} |

A is cyano

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-CF₃ |
| 2-F, 4-F, 6-F | 3-F, 4-CF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF_{3,} 4-Cl |
| 2-Cl | 3-CF_{3,} 4-Cl |
| 2-Br | 3-CF_{3,} 4-Cl |
| 2-Me | 3-CF_{3,} 4-Cl |
| 2-CF₃ | 3-CF_{3,} 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-OCF₃ |
| 2-F, 4-F, 6-F | 4-OCF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

A is NHC(O)Me

| | |
|---|---|
| 2-F | 2-F |
| 2-Cl | 2-F |
| 2-Br | 2-F |
| 2-Me | 2-F |
| 2-CF₃ | 2-F |
| 4-F | 2-F |
| 4-Cl | 2-F |
| 4-Br | 2-F |
| 4-NO₂ | 2-F |
| 2-F, 4-NO₂ | 2-F |
| 2-F, 4-F | 2-F |
| 2-Cl, 4-F | 2-F |
| 2-Me, 4-F | 2-F |
| 2-F, 6-F | 2-F |
| 2-F, 4-F, 6-F | 2-F |
| 2-F | 3-Br |
| 2-Cl | 3-Br |
| 2-Br | 3-Br |
| 2-Me | 3-Br |
| 2-CF₃ | 3-Br |
| 4-F | 3-Br |
| 4-Cl | 3-Br |
| 4-Br | 3-Br |
| 4-NO₂ | 3-Br |
| 2-F, 4-NO₂ | 3-Br |
| 2-F, 4-F | 3-Br |
| 2-Cl, 4-F | 3-Br |
| 2-Me, 4-F | 3-Br |
| 2-F, 6-F | 3-Br |
| 2-F, 4-F, 6-F | 3-Br |
| 2-F | 4-F |
| 2-Cl | 4-F |
| 2-Br | 4-F |
| 2-Me | 4-F |
| 2-CF₃ | 4-F |
| 4-F | 4-F |
| 4-Cl | 4-F |
| 4-Br | 4-F |
| 4-NO₂ | 4-F |
| 2-F, 4-NO₂ | 4-F |
| 2-F, 4-F | 4-F |
| 2-Cl, 4-F | 4-F |
| 2-Me, 4-F | 4-F |
| 2-F, 6-F | 4-F |
| 2-F, 4-F, 6-F | 4-F |
| 2-F | 4-CF₃ |
| 2-Cl | 4-CF₃ |
| 2-Br | 4-CF₃ |
| 2-Me | 4-CF₃ |
| 2-CF₃ | 4-CF₃ |
| 4-F | 4-CF₃ |
| 4-Cl | 4-CF₃ |
| 4-Br | 4-CF₃ |
| 4-NO₂ | 4-CF₃ |
| 2-F, 4-NO₂ | 4-CF₃ |
| 2-F, 4-F | 4-CF₃ |
| 2-Cl, 4-F | 4-CF₃ |
| 2-Me, 4-F | 4-CF₃ |
| 2-F, 6-F | 4-CF₃ |
| 2-F, 4-F, 6-F | 4-CF₃ |
| 2-F | 3-F, 4-Cl |
| 2-Cl | 3-F, 4-Cl |
| 2-Br | 3-F, 4-Cl |
| 2-Me | 3-F, 4-Cl |
| 2-CF₃ | 3-F, 4-Cl |
| 4-F | 3-F, 4-Cl |
| 4-Cl | 3-F, 4-Cl |
| 4-Br | 3-F, 4-Cl |
| 4-NO₂ | 3-F, 4-Cl |
| 2-F, 4-NO₂ | 3-F, 4-Cl |
| 2-F, 4-F | 3-F, 4-Cl |
| 2-Cl, 4-F | 3-F, 4-Cl |
| 2-Me, 4-F | 3-F, 4-Cl |
| 2-F, 6-F | 3-F, 4-Cl |
| 2-F, 4-F, 6-F | 3-F, 4-Cl |
| 2-F | 3-F, 4-OCF₃ |
| 2-Cl | 3-F, 4-OCF₃ |
| 2-Br | 3-F, 4-OCF₃ |
| 2-Me | 3-F, 4-OCF₃ |
| 2-CF₃ | 3-F, 4-OCF₃ |
| 4-F | 3-F, 4-OCF₃ |
| 4-Cl | 3-F, 4-OCF₃ |
| 4-Br | 3-F, 4-OCF₃ |
| 4-NO₂ | 3-F, 4-OCF₃ |
| 2-F, 4-NO₂ | 3-F, 4-OCF₃ |
| 2-F, 4-F | 3-F, 4-OCF₃ |
| 2-Cl, 4-F | 3-F, 4-OCF₃ |
| 2-Me, 4-F | 3-F, 4-OCF₃ |
| 2-F, 6-F | 3-F, 4-CF₃ |
| 2-F, 4-F, 6-F | 3-F, 4-CF₃ |
| 2-F | 3-Cl, 4-CF₃ |
| 2-Cl | 3-Cl, 4-CF₃ |
| 2-Br | 3-Cl, 4-CF₃ |
| 2-Me | 3-Cl, 4-CF₃ |
| 2-CF₃ | 3-Cl, 4-CF₃ |
| 4-F | 3-Cl, 4-CF₃ |
| 4-Cl | 3-Cl, 4-CF₃ |
| 4-Br | 3-Cl, 4-CF₃ |
| 4-NO₂ | 3-Cl, 4-CF₃ |
| 2-F, 4-NO₂ | 3-Cl, 4-CF₃ |
| 2-F, 4-F | 3-Cl, 4-CF₃ |
| 2-Cl, 4-F | 3-Cl, 4-CF₃ |
| 2-Me, 4-F | 3-Cl, 4-CF₃ |
| 2-F, 6-F | 3-Cl, 4-CF₃ |
| 2-F, 4-F, 6-F | 3-Cl, 4-CF₃ |
| 2-F | 3-CF₃, 4-Cl |
| 2-Cl | 3-CF₃, 4-Cl |
| 2-Br | 3-CF₃, 4-Cl |
| 2-Me | 3-CF₃, 4-Cl |
| 2-CF₃ | 3-CF₃, 4-Cl |
| 2-F | 3-F |
| 2-Cl | 3-F |
| 2-Br | 3-F |
| 2-Me | 3-F |
| 2-CF₃ | 3-F |
| 4-F | 3-F |
| 4-Cl | 3-F |
| 4-Br | 3-F |
| 4-NO₂ | 3-F |
| 2-F, 4-NO₂ | 3-F |
| 2-F, 4-F | 3-F |
| 2-Cl, 4-F | 3-F |
| 2-Me, 4-F | 3-F |
| 2-F, 6-F | 3-F |
| 2-F, 4-F, 6-F | 3-F |
| 2-F | 3-CF₃ |
| 2-Cl | 3-CF₃ |
| 2-Br | 3-CF₃ |
| 2-Me | 3-CF₃ |
| 2-CF₃ | 3-CF₃ |
| 4-F | 3-CF₃ |
| 4-Cl | 3-CF₃ |
| 4-Br | 3-CF₃ |
| 4-NO₂ | 3-CF₃ |
| 2-F, 4-NO₂ | 3-CF₃ |
| 2-F, 4-F | 3-CF₃ |
| 2-Cl, 4-F | 3-CF₃ |
| 2-Me, 4-F | 3-CF₃ |
| 2-F, 6-F | 3-CF₃ |
| 2-F, 4-F, 6-F | 3-CF₃ |
| 2-F | 4-Cl |
| 2-Cl | 4-Cl |
| 2-Br | 4-Cl |
| 2-Me | 4-Cl |
| 2-CF₃ | 4-Cl |
| 4-F | 4-Cl |
| 4-Cl | 4-Cl |
| 4-Br | 4-Cl |
| 4-NO₂ | 4-Cl |
| 2-F, 4-NO₂ | 4-Cl |
| 2-F, 4-F | 4-Cl |
| 2-Cl, 4-F | 4-Cl |
| 2-Me, 4-F | 4-Cl |
| 2-F, 6-F | 4-Cl |
| 2-F, 4-F, 6-F | 4-Cl |
| 2-F | 4-OCF₃ |
| 2-Cl | 4-OCF₃ |
| 2-Br | 4-OCF₃ |
| 2-Me | 4-OCF₃ |
| 2-CF₃ | 4-OCF₃ |
| 4-F | 4-OCF₃ |
| 4-Cl | 4-OCF₃ |
| 4-Br | 4-OCF₃ |
| 4-NO₂ | 4-OCF₃ |
| 2-F, 4-NO₂ | 4-OCF₃ |
| 2-F, 4-F | 4-OCF₃ |
| 2-Cl, 4-F | 4-OCF₃ |
| 2-Me, 4-F | 4-OCF₃ |
| 2-F, 6-F | 4-CF₃ |
| 2-F, 4-F, 6-F | 4-CF₃ |
| 2-F | 3-F, 4-Br |
| 2-Cl | 3-F, 4-Br |
| 2-Br | 3-F, 4-Br |
| 2-Me | 3-F, 4-Br |
| 2-CF₃ | 3-F, 4-Br |
| 4-F | 3-F, 4-Br |
| 4-Cl | 3-F, 4-Br |
| 4-Br | 3-F, 4-Br |
| 4-NO₂ | 3-F, 4-Br |
| 2-F, 4-NO₂ | 3-F, 4-Br |
| 2-F, 4-F | 3-F, 4-Br |
| 2-Cl, 4-F | 3-F, 4-Br |
| 2-Me, 4-F | 3-F, 4-Br |
| 2-F, 6-F | 3-F, 4-Br |
| 2-F, 4-F, 6-F | 3-F, 4-Br |
| 2-F | 3-Cl, 4-F |
| 2-Cl | 3-Cl, 4-F |
| 2-Br | 3-Cl, 4-F |
| 2-Me | 3-Cl, 4-F |
| 2-CF₃ | 3-Cl, 4-F |
| 4-F | 3-Cl, 4-F |
| 4-Cl | 3-Cl, 4-F |
| 4-Br | 3-Cl, 4-F |
| 4-NO₂ | 3-Cl, 4-F |
| 2-F, 4-NO₂ | 3-Cl, 4-F |
| 2-F, 4-F | 3-Cl, 4-F |
| 2-Cl, 4-F | 3-Cl, 4-F |
| 2-Me, 4-F | 3-Cl, 4-F |
| 2-F, 6-F | 3-Cl, 4-F |
| 2-F, 4-F, 6-F | 3-Cl, 4-F |
| 2-F | 3-Br, 4-F |
| 2-Cl | 3-Br, 4-F |
| 2-Br | 3-Br, 4-F |
| 2-Me | 3-Br, 4-F |
| 2-CF₃ | 3-Br, 4-F |
| 4-F | 3-Br, 4-F |
| 4-Cl | 3-Br, 4-F |
| 4-Br | 3-Br, 4-F |
| 4-NO₂ | 3-Br, 4-F |
| 2-F, 4-NO₂ | 3-Br, 4-F |
| 2-F, 4-F | 3-Br, 4-F |
| 2-Cl, 4-F | 3-Br, 4-F |
| 2-Me, 4-F | 3-Br, 4-F |
| 2-F, 6-F | 3-Br, 4-F |
| 2-F, 4-F, 6-F | 3-Br, 4-F |
| 4-F | 3-CF₃, 4-Cl |
| 4-Cl | 3-CF₃, 4-Cl |
| 4-Br | 3-CF₃, 4-Cl |
| 2-F, 4-F | 3-CF₃, 4-Cl |
| 2-Cl, 4-F | 3-CF₃, 4-Cl |
| 2-F | 3-Cl |
| 2-Cl | 3-Cl |
| 2-Br | 3-Cl |
| 2-Me | 3-Cl |
| 2-CF₃ | 3-Cl |
| 4-F | 3-Cl |
| 4-Cl | 3-Cl |
| 4-Br | 3-Cl |
| 4-NO₂ | 3-Cl |
| 2-F, 4-NO₂ | 3-Cl |
| 2-F, 4-F | 3-Cl |
| 2-Cl, 4-F | 3-Cl |
| 2-Me, 4-F | 3-Cl |
| 2-F, 6-F | 3-Cl |
| 2-F, 4-F, 6-F | 3-Cl |
| 2-F | 3-OCF₃ |
| 2-Cl | 3-OCF₃ |
| 2-Br | 3-OCF₃ |
| 2-Me | 3-OCF₃ |
| 2-CF₃ | 3-OCF₃ |
| 4-F | 3-OCF₃ |
| 4-Cl | 3-OCF₃ |
| 4-Br | 3-OCF₃ |
| 4-NO₂ | 3-OCF₃ |
| 2-F, 4-NO₂ | 3-OCF₃ |
| 2-F, 4-F | 3-OCF₃ |
| 2-Cl, 4-F | 3-OCF₃ |
| 2-Me, 4-F | 3-OCF₃ |
| 2-F, 6-F | 3-OCF₃ |
| 2-F, 4-F, 6-F | 3-OCF₃ |
| 2-F | 4-Br |
| 2-Cl | 4-Br |
| 2-Br | 4-Br |
| 2-Me | 4-Br |
| 2-CF₃ | 4-Br |
| 4-F | 4-Br |
| 4-Cl | 4-Br |
| 4-Br | 4-Br |
| 4-NO₂ | 4-Br |
| 2-F, 4-NO₂ | 4-Br |
| 2-F, 4-F | 4-Br |
| 2-Cl, 4-F | 4-Br |
| 2-Me, 4-F | 4-Br |
| 2-F, 6-F | 4-Br |
| 2-F, 4-F, 6-F | 4-Br |
| 2-F | 3-F, 4-F |
| 2-Cl | 3-F, 4-F |
| 2-Br | 3-F, 4-F |
| 2-Me | 3-F, 4-F |
| 2-CF₃ | 3-F, 4-F |
| 4-F | 3-F, 4-F |
| 4-Cl | 3-F, 4-F |
| 4-Br | 3-F, 4-F |
| 4-NO₂ | 3-F, 4-F |
| 2-F, 4-NO₂ | 3-F, 4-F |
| 2-F, 4-F | 3-F, 4-F |
| 2-Cl, 4-F | 3-F, 4-F |
| 2-Me, 4-F | 3-F, 4-F |
| 2-F, 6-F | 3-F, 4-F |
| 2-F, 4-F, 6-F | 3-F, 4-F |
| 2-F | 3-F, 4-CF₃ |
| 2-Cl | 3-F, 4-CF₃ |
| 2-Br | 3-F, 4-CF₃ |
| 2-Me | 3-F, 4-CF₃ |
| 2-CF₃ | 3-F, 4-CF₃ |
| 4-F | 3-F, 4-CF₃ |
| 4-Cl | 3-F, 4-CF₃ |
| 4-Br | 3-F, 4-CF₃ |
| 4-NO₂ | 3-F, 4-CF₃ |
| 2-F, 4-NO₂ | 3-F, 4-CF₃ |
| 2-F, 4-F | 3-F, 4-CF₃ |
| 2-Cl, 4-F | 3-F, 4-CF₃ |
| 2-Me, 4-F | 3-F, 4-CF₃ |
| 2-F, 6-F | 3-F, 4-CF₃ |
| 2-F, 4-F, 6-F | 3-F, 4-CF₃ |
| 2-F | 3-Cl, 4-Cl |
| 2-Cl | 3-Cl, 4-Cl |
| 2-Br | 3-Cl, 4-Cl |
| 2-Me | 3-Cl, 4-Cl |
| 2-CF₃ | 3-Cl, 4-Cl |
| 4-F | 3-Cl, 4-Cl |
| 4-Cl | 3-Cl, 4-Cl |
| 4-Br | 3-Cl, 4-Cl |
| 4-NO₂ | 3-Cl, 4-Cl |
| 2-F, 4-NO₂ | 3-Cl, 4-Cl |
| 2-F, 4-F | 3-Cl, 4-Cl |
| 2-Cl, 4-F | 3-Cl, 4-Cl |
| 2-Me, 4-F | 3-Cl, 4-Cl |
| 2-F, 6-F | 3-Cl, 4-Cl |
| 2-F, 4-F, 6-F | 3-Cl, 4-Cl |
| 2-F | 3-CF₃, 4-F |
| 2-Cl | 3-CF₃, 4-F |
| 2-Br | 3-CF₃, 4-F |
| 2-Me | 3-CF₃, 4-F |
| 2-CF₃ | 3-CF₃, 4-F |
| 4-F | 3-CF₃, 4-F |
| 4-Cl | 3-CF₃, 4-F |
| 4-Br | 3-CF₃, 4-F |
| 4-NO₂ | 3-CF₃, 4-F |
| 2-F, 4-NO₂ | 3-CF₃, 4-F |
| 2-F, 4-F | 3-CF₃, 4-F |
| 2-Cl, 4-F | 3-CF₃, 4-F |
| 2-Me, 4-F | 3-CF₃, 4-F |
| 2-F, 6-F | 3-CF₃, 4-F |
| 2-F, 4-F, 6-F | 3-CF₃, 4-F |
| 2-Me, 4-F | 3-CF₃, 4-Cl |
| 2-F, 6-F | 3-CF₃, 4-Cl |
| 2-F, 4-F, 6-F | 3-CF₃, 4-Cl |

TABLE 17

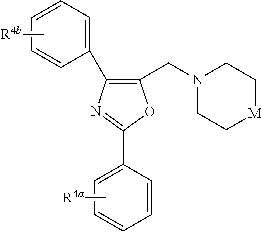

M is O

| R^{4b} | R^{4a} |
|---|---|
| 2-F | 3-Cl |
| 2-F | 4-Cl |
| 2-F | 3-CF₃ |
| 2-F | 4-CF₃ |
| 2-F | 4-OCF₃ |
| 2-F | 3-Cl, 4-Cl |
| 2-F | 3-Cl, 4-F |
| 2-F | 3-Cl, 4-CF₃ |
| 2-F | 3-CF₃, 4-Cl |
| 2-Me | 3-Cl |
| 2-Me | 4-F |
| 2-Me | 3-CF₃ |
| 2-Me | 4-CF₃ |
| 2-Me | 4-OCF₃ |
| 2-Me | 3-Cl, 4-Cl |
| 2-Me | 3-Cl, 4-F |
| 2-Me | 3-Cl, 4-CF₃ |
| 2-Me | 3-CF₃, 4-Cl |
| 2-F, 6-F | 3-Cl |
| 2-F, 6-F | 4-F |
| 2-F, 6-F | 3-CF₃ |
| 2-F, 6-F | 4-CF₃ |
| 2-F, 6-F | 4-OCF₃ |
| 2-F, 6-F | 3-Cl, 4-Cl |
| 2-Cl | 3-Cl |
| 2-Cl | 4-Cl |
| 2-Cl | 3-CF₃ |
| 2-Cl | 4-CF₃ |
| 2-Cl | 4-OCF₃ |
| 2-Cl | 3-Cl, 4-Cl |
| 2-Cl | 3-Cl, 4-F |
| 2-Cl | 3-Cl, 4-CF₃ |
| 2-Cl | 3-CF₃, 4-Cl |
| 4-F | 3-Cl |
| 4-F | 4-F |
| 4-F | 3-CF₃ |
| 4-F | 4-CF₃ |
| 4-F | 4-OCF₃ |
| 4-F | 3-Cl, 4-Cl |
| 4-F | 3-Cl, 4-F |
| 4-F | 3-Cl, 4-CF₃ |
| 4-F | 3-CF₃, 4-Cl |
| 2-F, 6-F | 3-Cl, 4-F |
| 2-F, 6-F | 3-Cl, 4-CF₃ |
| 2-F, 6-F | 3-CF₃, 4-Cl |
| 2-F, 4-F, 6-F | 3-Cl |
| 2-F, 4-F, 6-F | 4-F |
| 2-F, 4-F, 6-F | 3-CF₃ |
| 2-Br | 3-Cl |
| 2-Br | 4-Cl |
| 2-Br | 3-CF₃ |
| 2-Br | 4-CF₃ |
| 2-Br | 4-OCF₃ |
| 2-Br | 3-Cl, 4-Cl |
| 2-Br | 3-Cl, 4-F |
| 2-Br | 3-Cl, 4-CF₃ |
| 2-Br | 3-CF₃, 4-Cl |
| 2-F, 4-F | 3-Cl |
| 2-F, 4-F | 4-F |
| 2-F, 4-F | 3-CF₃ |
| 2-F, 4-F | 4-CF₃ |
| 2-F, 4-F | 4-OCF₃ |
| 2-F, 4-F | 3-Cl, 4-Cl |
| 2-F, 4-F | 3-Cl, 4-F |
| 2-F, 4-F | 3-Cl, 4-CF₃ |
| 2-F, 4-F | 3-CF₃, 4-Cl |
| 2-F, 4-F, 6-F | 4-CF₃ |
| 2-F, 4-F, 6-F | 4-OCF₃ |
| 2-F, 4-F, 6-F | 3-Cl, 4-Cl |
| 2-F, 4-F, 6-F | 3-Cl, 4-F |
| 2-F, 4-F, 6-F | 3-Cl, 4-CF₃ |
| 2-F, 4-F, 6-F | 3-CF₃, 4-Cl |

A compound of this invention will generally be used as an invertebrate pest control active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids can be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which are branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention can also contain formulation auxiliaries and additives, known to those skilled in the art as formulation aids (some of which can be considered to also function as solid diluents, liquid diluents or surfactants). Such formulation auxiliaries and additives can control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all formulations are prepared in conventional ways. Compound numbers refer to compounds in Index Tables A-N. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| compound 36 | 98.5% |
| silica aerogel | 0.5% |
| synthetic amorphous fine silica | 1.0% |

### Example B

Wettable Powder

| | |
|---|---|
| compound 86 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| compound 90 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| compound 100 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| compound 112 | 10.0% |
| polyoxyethylene sorbitol hexoleate | 20.0% |
| C₆-C₁₀ fatty acid methyl ester | 70.0% |

### Example F

Microemulsion

| | |
|---|---|
| compound 113 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| compound 119 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### Example H

Fertilizer Stick

| | |
|---|---|
| compound 136 | 2.50% |
| pyrrolidone-styrene copolymer | 4.80% |
| tristyrylphenyl 16-ethoxylate | 2.30% |
| talc | 0.80% |
| corn starch | 5.00% |
| slow-release fertilizer | 36.00% |
| kaolin | 38.00% |
| water | 10.60% |

### Example I

Suspension Concentrate

| | |
|---|---|
| compound 137 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example J

Emulsion in Water

| | |
|---|---|
| compound 138 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example K

Oil Dispersion

| | |
|---|---|
| compound 141 | 25% |
| polyoxyethylene sorbitol hexaoleate | 15% |
| organically modified bentonite clay | 2.5% |
| fatty acid methyl ester | 57.5% |

### Example L

Suspoemulsion

| | |
|---|---|
| compound 152 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Compounds of this invention exhibit activity against a wide spectrum of invertebrate pests. These pests include invertebrates inhabiting a variety of environments such as, for example, plant foliage, roots, soil, harvested crops or other foodstuffs, building structures or animal integuments. These pests include, for example, invertebrates feeding on foliage (including leaves, stems, flowers and fruits), seeds, wood, textile fibers or animal blood or tissues, and thereby causing injury or damage to, for example, growing or stored agronomic crops, forests, greenhouse crops, ornamentals, nursery crops, stored foodstuffs or fiber products, or houses or other structures or their contents, or being harmful to animal health or public health. Those skilled in the art will appreciate that not all compounds are equally effective against all growth stages of all pests.

These present compounds and compositions are thus useful agronomically for protecting field crops from phytophagous invertebrate pests, and also nonagronomically for protecting other horticultural crops and plants from phytophagous invertebrate pests. This utility includes protecting crops and other plants (i.e. both agronomic and nonagronomic) that contain genetic material introduced by genetic engineering (i.e. transgenic) or modified by mutagenesis to provide advantageous traits. Examples of such traits include tolerance to herbicides, resistance to phytophagous pests (e.g., insects, mites, aphids, spiders, nematodes, snails, plant-pathogenic fungi, bacteria and viruses), improved plant growth, increased tolerance of adverse growing conditions such as high or low temperatures, low or high soil moisture, and high salinity, increased flowering or fruiting, greater harvest yields, more rapid maturation, higher quality and/or nutritional value of the harvested product, or improved storage or process properties of the harvested products. Transgenic plants can be modified to express multiple traits. Examples of plants containing traits provided by genetic engineering or mutagenesis include varieties of corn, cotton, soybean and potato expressing an insecticidal Bacillus thuringiensis toxin such as YIELD GARD®, KNOCKOUT®, STARLINK®, BOLLGARD®, NuCOTN® and NEWLEAF®, and herbicide-tolerant varieties of corn, cotton, soybean and rapeseed such as ROUNDUP READY®, LIBERTY LINK®, IMI®, STS® and CLEARFIELD®, as well as crops expressing N-acetyltransferase (GAT) to provide resistance to glyphosate herbicide, or crops containing the HRA gene providing resistance to herbicides inhibiting acetolactate synthase (ALS). The present compounds and compositions may interact synergistically with traits introduced by genetic engineering or modified by mutagenesis, thus enhancing phenotypic expression or effectiveness of the traits or increasing the invertebrate pest control effectiveness of the present compounds and compositions. In particular, the present compounds and compositions may interact synergistically with the phenotypic expression of proteins or other natural products toxic to invertebrate pests to provide greater-than-additive control of these pests.

Compositions of this invention can also optionally comprise plant nutrients, e.g., a fertilizer composition comprising at least one plant nutrient selected from nitrogen, phosphorus, potassium, sulfur, calcium, magnesium, iron, copper, boron, manganese, zinc, and molybdenum. Of note are compositions comprising at least one fertilizer composition comprising at least one plant nutrient selected from nitrogen, phosphorus, potassium, sulfur, calcium and magnesium. Compositions of the present invention which further comprise at least one plant nutrient can be in the form of liquids or solids. Of note are solid formulations in the form of granules, small sticks or tablets. Solid formulations comprising a fertilizer composition can be prepared by mixing the compound or composition of the present invention with the fertilizer composition together with formulating ingredients and then preparing the formulation by methods such as granulation or extrusion. Alternatively solid formulations can be prepared by spraying a solution or suspension of a compound or composition of the present invention in a volatile solvent onto a previous prepared fertilizer composition in the form of dimensionally stable mixtures, e.g., granules, small sticks or tablets, and then evaporating the solvent.

Examples of agronomic or nonagronomic invertebrate pests include eggs, larvae and adults of the order Lepidoptera, such as armyworms, cutworms, loopers, and heliothines in the family Noctuidae (e.g., pink stem borer (Sesamia inferens Walker), corn stalk borer (Sesamia nonagrioides Lefebvre), southern armyworm (Spodoptera eridania Cramer), fall armyworm (Spodoptera fugiperda J. E. Smith), beet armyworm (Spodoptera exigua Hübner), cotton leafworm (Spodoptera littoralis Boisduval), yellowstriped armyworm (Spodoptera ornithogalli Guenée), black cutworm (Agrotis ipsilon Hufnagel), velvetbean caterpillar (Anticarsia gemmatalis Hübner), green fruitworm (Lithophane antennata Walker), cabbage armyworm (Barathra brassicae Linnaeus), soybean looper (Pseudoplusia includens Walker), cabbage looper (Trichoplusia ni Hübner), tobacco budworm (Heliothis virescens Fabricius)); borers, casebearers, webworms, coneworms, cabbageworms and skeletonizers from the family Pyralidae (e.g., European corn borer (Ostrinia nubilalis Hübner), navel orangeworm (Amyelois transitella Walker), corn root webworm (Crambus caliginosellus Clemens), sod webworms (Pyralidae: Crambinae) such as sod worm (Herpetogramma licarsisalis Walker), sugarcane stem borer (Chilo infuscatellus Snellen), tomato small borer (Neoleucinodes elegantalis Guenée), green leafroller (Cnaphalocerus medinalis), grape leaffolder (Desmia funeralis Hübner), melon worm (Diaphania nitidalis Stoll), cabbage center grub (Helluala hydralis Guenée), yellow stem borer (Scirpophaga incertulas Walker), early shoot borer (Scirpophaga infuscatellus Snellen), white stem borer (Scirpophaga innotata Walker), top shoot borer (Scirpophaga nivella Fabricius), dark-headed rice borer (Chilo polychrysus Meyrick), cabbage cluster caterpillar (Crocidolomia binotalis English)); leafrollers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck), citrus false codling moth (Cryptophlebia leucotreta Meyrick), citrus borer (Ecdytolopha aurantiana Lima), redbanded leafroller (Argyrotaenia velutinana Walker), obliquebanded leafroller (Choristoneura rosaceana Harris), light brown apple moth (Epiphyas postvittana Walker), European grape berry moth (Eupoecilia ambiguella Hübner), apple bud moth (Pandemis pyrusana Kearfott), omnivorous leafroller (Platynota stultana Walsingham), barred fruit-tree tortrix (Pandemis cerasana Hübner), apple brown tortrix (Pandemis heparana Denis & Schiffermüller)); and many other economically important lepidoptera (e.g., diamondback moth (Plutella xylostella Linnaeus), pink bollworm (Pectinophora gossypiella Saunders), gypsy moth (Lymantria dispar Linnaeus), peach fruit borer (Carposina niponensis Walsingham), peach twig borer (Anarsia lineatella Zeller), potato tuberworm (Phthorimaea operculella Zeller), spotted teniform leafminer (Lithocolletis blancardella Fabricius), Asiatic apple leafminer (Lithocolletis ringoniella Matsumura), rice leaffolder (Lerodea eufala Edwards), apple leafminer (Leucoptera scitella Zeller)); eggs, nymphs and adults of the order Blattodea including cockroaches from the families Blattellidae and Blattidae (e.g., oriental cockroach (Blatta orientalis Linnaeus), Asian cockroach (Blatella asahinai Mizukubo), German cockroach (Blattella germanica Linnaeus), brownbanded cockroach (Supella longipalpa Fabricius), American cockroach (Periplaneta americana Linnaeus), brown cockroach (Periplaneta brunnea Burmeister), Madeira cockroach (Leucophaea maderae Fabricius)), smoky brown cockroach (Periplaneta fuliginosa Service), Australian Cockroach (Periplaneta australasiae Fabr.), lobster cockroach (Nauphoeta cinerea Olivier) and smooth cockroach (Symploce pallens Stephens)); eggs, foliar feeding, fruit feeding, root feeding, seed feeding and vesicular tissue feeding larvae and adults of the order Coleoptera including weevils from the families Anthribidae, Bruchidae, and Curculionidae (e.g., boll weevil (Anthonomus grandis Boheman), rice water weevil (Lissorhoptrus orzophilus Kuschel), granary weevil (Sitophilus granarius Linnaeus), rice weevil (Sitophilus oryzae Linnaeus)), annual bluegrass weevil (Listronotus maculicollis Dietz), bluegrass billbug (Sphenophorus parvulus Gyllenhal), hunting billbug (Sphenophorus venatus vestitus), Denver billbug (Sphenophorus cicatristriatus Fahraeus)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scarabaeidae (e.g., Japanese beetle (Popillia japonica Newman), oriental beetle (Anomala orientalis Waterhouse, Exomala orientalis (Waterhouse) Baraud), northern masked chafer (Cyclocephala borealis Arrow), southern masked chafer (Cyclocephala immaculata Olivier or C. lurida Bland), dung beetle and white grub (Aphodius spp.), black turfgrass ataenius (Ataenius spretulus Haldeman), green June beetle (Cotinis nitida Linnaeus), Asiatic garden beetle (Maladera castanea Arrow), May/June beetles (Phyllophaga spp.) and European chafer (Rhizotrogus majalis Razoumowsky)); carpet beetles from the family Dermestidae; wireworms from the family Elateridae; bark beetles from the family Scolytidae and flour beetles from the family Tenebrionidae.

In addition, agronomic and nonagronomic pests include: eggs, adults and larvae of the order Dermaptera including earwigs from the family Forficulidae (e.g., European earwig (Forficula auricularia Linnaeus), black earwig (Chelisoches morio Fabricius)); eggs, immatures, adults and nymphs of the orders Hemiptera and Homoptera such as, plant bugs from the family Miridae, cicadas from the family Cicadidae, leafhoppers (e.g. Empoasca spp.) from the family Cicadellidae, bed bugs (e.g., Cimex lectularius Linnaeus) from the family Cimicidae, planthoppers from the families Fulgoroidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, chinch bugs (e.g., hairy chinch bug (Blissus leucopterus hirtus Montandon) and southern chinch bug (Blissus insularis Barber)) and other seed bugs from the family Lygaeidae, spittlebugs from the family Cercopidae squash bugs from the family Coreidae, and red bugs and cotton stainers from the family Pyrrhocoridae.

Agronomic and nonagronomic pests also include: eggs, larvae, nymphs and adults of the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)); flat mites in the family Tenuipalpidae (e.g., citrus flat mite (Brevipalpus lewisi McGregor)); rust and bud mites in the family Eriophyidae and other foliar feeding mites and mites important in human and animal health, i.e. dust mites in the family Epidermoptidae, follicle mites in the family Demodicidae, grain mites in the family Glycyphagidae; ticks in the family Ixodidae, commonly known as hard ticks (e.g., deer tick (Ixodes scapularis Say), Australian paralysis tick (Ixodes holocyclus Neumann), American dog tick (Dermacentor variabilis Say), lone star tick (Amblyomma americanum Linnaeus)) and ticks in the family Argasidae, commonly known as soft ticks (e.g., relapsing fever tick (Ornithodoros turicata), common fowl tick (Argas radiatus)); scab and itch mites in the families Psoroptidae, Pyemotidae, and Sarcoptidae; eggs, adults and immatures of the order Orthoptera including grasshoppers, locusts and crickets (e.g., migratory grasshoppers (e.g., Melanoplus sanguinipes Fabricius, M. differentialis Thomas), American grasshoppers (e.g., Schistocerca americana Drury), desert locust (Schistocerca gregaria Forskal), migratory locust (Locusta migratoria Linnaeus), bush locust (Zonocerus spp.), house cricket (Acheta domesticus Linnaeus), mole crickets (e.g., tawny mole cricket (Scapteriscus vicinus Scudder) and southern mole cricket (Scapteriscus borellii Giglio-Tos)); eggs, adults and immatures of the order Diptera including leafminers (e.g., Liriomyza spp. such as serpentine vegetable leafminer (Liriomyza sativae Blanchard)), midges, fruit flies (Tephritidae), flit flies (e.g., Oscinella frit Linnaeus), soil maggots, house flies (e.g., Musca domestica Linnaeus), lesser house flies (e.g., Fannia canicularis Linnaeus, F. femoralis Stein), stable flies (e.g., Stomoxys calcitrans Linnaeus), face flies, horn flies, blow flies (e.g., Chrysomya spp., Phormia spp.), and other muscoid fly pests, horse flies (e.g., Tabanus spp.), bot flies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; eggs, adults and immatures of the order Thysanoptera including onion thrips (Thrips tabaci Lindeman), flower thrips (Frankliniella spp.), and other foliar feeding thrips; insect pests of the order Hymenoptera including ants of the Family Formicidae including the Florida carpenter ant (Camponotus floridanus Buckley), red carpenter ant (Camponotus ferrugineus Fabricius), black carpenter ant (Camponotus pennsylvanicus De Geer), white-footed ant (Technomyrmex albipes fr. Smith), big headed ants (Pheidole sp.), ghost ant (Tapinoma melanocephalum Fabricius); Pharaoh ant (Monomorium pharaonis Linnaeus), little fire ant (Wasmannia auropunctata Roger), fire ant (Solenopsis geminata Fabricius), red imported fire ant (Solenopsis invicta Buren), Argentine ant (Iridomyrmex humilis Mayr), crazy ant (Paratrechina longicornis Latreille), pavement ant (Tetramorium caespitum Linnaeus), cornfield ant (Lasius alienus Förster) and odorous house ant (Tapinoma sessile Say). Other Hymenoptera including bees (including carpenter bees), hornets, yellow jackets, wasps, and sawflies (Neodiprion spp.; Cephus spp.); insect pests of the order Isoptera including termites in the Termitidae (e.g., Macrotermes sp., Odontotermes obesus Rambur), Kalotermitidae (e.g., Cryptotermes sp.), and Rhinotermitidae (e.g., Reticulitermes sp., Coptolermes sp., Heterotermes tenuis Hagen) families, the eastern subterranean termite (Reticulitermes flavipes Kollar), western subterranean termite (Reticulitermes hesperus Banks), Formosan subterranean termite (Coptotermes formosanus Shiraki), West Indian drywood termite (Incisitermes immigrans Snyder), powder post termite (Cryptotermes brevis Walker), drywood termite (Incisitermes snyderi Light), southeastern subterranean termite (Reticulitermes virginicus Banks), western drywood termite (Incisitermes minor Hagen), arboreal termites such as Nasutilermes sp. and other termites of economic importance; insect pests of the order Thysanura such as silverfish (Lepisma saccharina Linnaeus) and firebrat (Thermobia domestica Packard); insect pests of the order Mallophaga and including the head louse (Pediculus humanus capitis De Geer), body louse (Pediculus humanus Linnaeus), chicken body louse (Menacanthus stramineus Nitszch), dog biting louse (Trichodectes canis De Geer), fluff louse (Goniocotes gallinae De Geer), sheep body louse (Bovicola ovis Schrank), short-nosed cattle louse (Haematopinus eurysternus Nitzsch), long-nosed cattle louse (Linognathus vituli Linnaeus) and other sucking and chewing parasitic lice that attack man and animals; insect pests of the order Siphonoptera including the oriental rat flea (Xenopsylla cheopis Rothschild), cat flea (Ctenocephalides felis Bouche), dog flea (Ctenocephalides canis Curtis), hen flea (Ceratophyllus gallinae Schrank), sticktight flea (Echidnophaga gallinacea Westwood), human flea (Pulex irritans Linnaeus) and other fleas afflicting mammals and birds. Additional arthropod pests covered include: spiders in the order Araneae such as the brown recluse spider (Loxosceles reclusa Gertsch & Mulaik) and the black widow spider (Latrodectus mactans Fabricius), and centipedes in the order Scutigeromorpha such as the house centipede (Scutigera coleoptrata Linnaeus).

Examples of invertebrate pests of stored grain include larger grain borer (Prostephanus truncatus), lesser grain borer (Rhyzopertha dominica), rice weevil (Stiophilus oryzae), maize weevil (Stiophilus zeamais), cowpea weevil (Callosobruchus maculatus), red flour beetle (Tribolium castaneum), granary weevil (Stiophilus granarius), Indian meal moth (Plodia interpunctella), Mediterranean flour beetle (Ephestia kuhniella) and flat or rusty grain beetle (Cryptolestis ferrugineus).

Compounds of the invention show particularly high activity against pests in the order Lepidoptera (e.g., Alabama argillacea Hübner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenée (rice leaf roller), Crambus caliginosellus Clemens (corn root webworm), Crambus teterrellus Zincken (bluegrass webworm), Cydia pomonella Linnaeus (codling moth), Earias insulana Boisduval (spiny bollworm), Earias vittella Fabricius (spotted bollworm), Helicoverpa armigera Hübner (American bollworm), Helicoverpa zea Boddie (corn earworm), Heliothis virescens Fabricius (tobacco budworm), Herpetogramma licarsisalis Walker (sod webworm), Lobesia botrana Denis & Schiffermüller (grape berry moth), Pectinophora gossypiella Saunders (pink bollworm), Phyllocnistis citrella Stainton (citrus leafminer), Pieris brassicae Linnaeus (large white butterfly), Pieris rapae Linnaeus (small white butterfly), Plutella xylostella Linnaeus (diamondback moth), Spodoptera exigua Hübner (beet armyworm), Spodoptera litura Fabricius (tobacco cutworm, cluster caterpillar), Spodoptera frugiperda J. E. Smith (fall armyworm), Trichoplusia ni Hübner (cabbage looper) and Tuta absoluta Meyrick (tomato leafminer)).

Compounds of the invention also have activity on members from the order Homoptera including: Acyrthosiphon pisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Paaserini (rosy apple aphid), Eriosoma lanigerum Hausmann (woolly apple aphid), Hyalopterus pruni Geoffroy (mealy plum aphid), Lipaphis erysimi Kaltenbach (turnip aphid), Metopolophium dirrhodum Walker (cereal aphid), Macrosiphum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graminum Rondani (greenbug), Sitobion avenae Fabricius (English grain aphid), Therioaphis maculata Buckton (spotted alfalfa aphid), Toxoptera aurantii Boyer de Fonscolombe (black citrus aphid), and Toxoptera citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifolii Bellows & Perring (silverleaf whitefly), Dialeurodes citri Ashmead (citrus whitefly) and Trialeurodes vaporariorum Westwood (greenhouse whitefly); Empoasca fabae Harris (potato leafhopper), Laodelphax striatellus Fallen (smaller brown planthopper), Macrolestes quadrilineatus Forbes (aster leafhopper), Nephotettix cinticeps Uhler (green leafhopper), Nephotettix nigropictus Stäl (rice leafhopper), Nilaparvata lugens Stäl (brown planthopper), Peregrinus maidis Ashmead (corn planthopper), Sogatella furcifera Horvath (white-backed planthopper), Sogatodes orizicola Muir (rice delphacid), Typhlocyba pomaria McAtee white apple leafhopper, Erylhroneoura spp. (grape leafhoppers); Magicidada septendecim Linnaeus (periodical cicada); Icerya purchasi Maskell (cottony cushion scale), Quadraspidiotus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persimmon psylla).

Compounds of this invention may also have activity on members from the order Hemiptera including: Acrosternum hilare Say (green stink bug), Anasa tristis De Geer (squash bug), Blissus leucopterus leucopterus Say (chinch bug), Cimex lectularius Linnaeus (bed bug) Corythuca gossypii Fabricius (cotton lace bug), Cyrtopeltis modesta Distant (tomato bug), Dysdercus suturellus Herrich-Schäiffer (cotton stainer), Euchistus servus Say (brown stink bug), Euchistus variolarius Palisot de Beauvois (one-spotted stink bug), Graptosthetus spp. (complex of seed bugs), Leptoglossus corculus Say (leaf-footed pine seed bug), Lygus lineolaris Palisot de Beauvois (tarnished plant bug), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper). Other insect orders controlled by compounds of the invention include Thysanoptera (e.g., Frankliniella occidentalis Pergande (western flower thrips), Scirthothrips citri Moulton (citrus thrips), Sericothrips variabilis Beach (soybean thrips), and Thrips tabaci Lindeman (onion thrips); and the order Coleoptera (e.g., Leptinotarsa decemlineata Say (Colorado potato beetle), Epilachna varivestis Mulsant (Mexican bean beetle) and wireworms of the genera Agriotes, Athous or Limonius).

Note that some contemporary classification systems place Homoptera as a suborder within the order Hemiptera.

Of note is use of compounds of this invention for controlling diamondback moth (Plutella xylostella). Of note is use of compounds of this invention for controlling fall armyworm (Spodoptera frugiperda).

Compounds of this invention can also be mixed with one or more other biologically active compounds or agents including insecticides, fungicides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, other biologically active compounds or entomopathogenic bacteria, virus or fungi to form a multi-component pesticide giving an even broader spectrum of agronomic and nonagronomic utility. Thus the present invention also pertains to a composition comprising a biologically effective amount of a compound of Formula 1, an N-oxide, or salt thereof, at least one additional component selected from the group consisting of surfactants, solid diluents and liquid diluents, and at least one additional biologically active compound or agent. For mixtures of the present invention, the other biologically active compounds or agents can be formulated together with the present compounds, including the compounds of Formula 1, to form a premix, or the other biologically active compounds or agents can be formulated separately from the present compounds, including the compounds of Formula 1, and the two formulations combined together before application (e.g., in a spray tank) or, alternatively, applied in succession.

Examples of such biologically active compounds or agents with which compounds of this invention can be formulated are insecticides such as abamectin, acephate, acequinocyl, acetamiprid, acrinathrin, amidoflumet, amitraz, avermectin, azadirachtin, azinphos-methyl, benfuracarb, bensultap, bifenthrin, bifenazate, bistrifluoron, borate, buprofezin, cadusafos, carbaryl, carbofuran, cartap, carzol, chlorantraniliprole, chlorfenapyr, chlorfluazuron, chlorpyrifos, chlorpyri fos-methyl, chromafenozide, clofentezin, clothianidin, cyantraniliprole, cycloprothrin, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenitrothion, fenothiocarb, fenoxycarb, fenpropathrin, fenvalerate, fipronil, flonicamid, flubendiamide, flucythrinate, flufenerim, flufenoxuron, fluvalinate, tau-fluvalinate, fonophos, formetanate, fosthiazate, halofenozide, hexaflumuron, hexythiazox, hydramethylnon, imidacloprid, indoxacarb, insecticidal soaps, isofenphos, lufenuron, malathion, meperfluthrin, metaflumizone, metaldehyde, methamidophos, methidathion, methiodicarb, methomyl, methoprene, methoxychlor, metofluthrin, monocrotophos, methoxyfenozide, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyrethrin, pyridaben, pyridalyl, pyrifluquinazon, pyriprole, pyriproxyfen, rotenone, ryanodine, silafluofen, spinetoram, spinosad, spirodiclofen, spiromesifen, spirotetramat, sulprofos, sulfoxaflor, tebufenozide, tebufenpyrad, teflubenzuron, tefluthrin, terbufos, tetrachlorvinphos, tetramethrin, tetramethylfluthrin, thiacloprid, thiamethoxam, thiodicarb, thiosultap-sodium, tolfenpyrad, tralomethrin, triazamate, trichlorfon, triflumuron, Bacillus thuringiensis delta-endotoxins, entomopathogenic bacteria, entomopathogenic viruses and entomopathogenic fungi.

Of note are insecticides such as abamectin, acetamiprid, acrinathrin, amitraz, avermectin, azadirachtin, bensultap, bifenthrin, buprofezin, cadusafos, carbaryl, cartap, chlorantraniliprole, chlorfenapyr, chlorpyrifos, clothianidin, cyantraniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, flufenoxuron, fluvalinate, formetanate, fosthiazate, hexaflumuron, hydramethylnon, imidacloprid, indoxacarb, lufenuron, metaflumizone, methiodicarb, methomyl, methoprene, methoxyfenozide, nitenpyram, nithiazine, novaluron, oxamyl, pymetrozine, pyrethrin, pyridaben, pyridalyl, pyriproxyfen, ryanodine, spinetoram, spinosad, spirodiclofen, spiromesifen, spirotetramat, tebufenozide, tetramethrin, thiacloprid, thiamethoxam, thiodicarb, thiosultap-sodium, tralomethrin, triazamate, triflumuron, Bacillus thuringiensis delta-endotoxins, all strains of Bacillus thuringiensis and all strains of Nucleo polyhedrosis viruses.

One embodiment of biological agents for mixing with compounds of this invention include entomopathogenic bacteria such as Bacillus thuringiensis, and the encapsulated delta-endotoxins of Bacillus thuringiensis such as MVP® and MVPII® bioinsecticides prepared by the CellCap® process (CellCap®, MVP® and MVPII® are trademarks of Mycogen Corporation, Indianapolis, Ind., USA); entomopathogenic fungi such as green muscardine fungus; and entomopathogenic (both naturally occurring and genetically modified) viruses including baculovirus, nucleopolyhedro virus (NPV) such as Helicoverpa zea nucleopolyhedrovirus (HzNPV), Anagrapha falcifera nucleopolyhedrovirus (AfNPV); and granulosis virus (GV) such as Cydia pomonella granulosis virus (CpGV).

Of particular note is such a combination where the other invertebrate pest control active ingredient belongs to a different chemical class or has a different site of action than the compound of Formula 1. In certain instances, a combination with at least one other invertebrate pest control active ingredient having a similar spectrum of control but a different site of action will be particularly advantageous for resistance management. Thus, a composition of the present invention can further comprise a biologically effective amount of at least one additional invertebrate pest control active ingredient having a similar spectrum of control but belonging to a different chemical class or having a different site of action. These additional biologically active compounds or agents include, but are not limited to, sodium channel modulators such as bifenthrin, cypermethrin, cyhalothrin, lambda-cyhalothrin, cyfluthrin, beta-cyfluthrin, deltamethrin, dimefluthrin, esfenvalerate, fenvalerate, indoxacarb, metofluthrin, profluthrin, pyrethrin and tralomethrin; cholinesterase inhibitors such as chlorpyrifos, methomyl, oxamyl, thiodicarb and triazamate; neonicotinoids such as acetamiprid, clothianidin, dinotefuran, imidacloprid, nitenpyram, nithiazine, thiacloprid and thiamethoxam; insecticidal macrocyclic lactones such as spinetoram, spinosad, abamectin, avermectin and emamectin; GABA (γ-aminobutyric acid)-gated chloride channel antagonists such as avermectin or blockers such as ethiprole and fipronil; chitin synthesis inhibitors such as buprofezin, cyromazine, flufenoxuron, hexaflumuron, lufenuron, novaluron, noviflumuron and triflumuron; juvenile hormone mimics such as diofenolan, fenoxycarb, methoprene and pyriproxyfen; octopamine receptor ligands such as amitraz; molting inhibitors and ecdysone agonists such as azadirachtin, methoxyfenozide and tebufenozide; ryanodine receptor ligands such as ryanodine, anthranilic diamides such as chlorantraniliprole, cyantraniliprole and flubendiamide; nereistoxin analogs such as cartap; mitochondrial electron transport inhibitors such as chlorfenapyr, hydramethylnon and pyridaben; lipid biosynthesis inhibitors such as spirodiclofen and spiromesifen; cyclodiene insecticides such as dieldrin or endosulfan; pyrethroids; carbamates; insecticidal ureas; and biological agents including nucleopolyhedro viruses (NPV), members of Bacillus thuringiensis, encapsulated delta-endotoxins of Bacillus thuringiensis, and other naturally occurring or genetically modified insecticidal viruses.

Further examples of biologically active compounds or agents with which compounds of this invention can be formulated are: fungicides such as 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, acibenzolar, aldimorph, amisulbrom, azaconazole, azoxystrobin, benalaxyl, benomyl, benthiavalicarb, benthiavalicarb-isopropyl, binomial, biphenyl, bitertanol, blasticidin-S, Bordeaux mixture (Tribasic copper sulfate), boscalid/nicobifen, bromuconazole, bupirimate, buthiobate, carboxin, carpropamid, captafol, captan, carbendazim, chloroneb, chlorothalonil, chlozolinate, clotrimazole, copper oxychloride, copper salts such as copper sulfate and copper hydroxide, cyazofamid, cyflunamid, cymoxanil, cyproconazole, cyprodinil, dichlofluanid, diclocymet, diclomezine, dicloran, diethofencarb, difenoconazole, dimethomorph, dimoxystrobin, diniconazole, diniconazole-M, dinocap, discostrobin, dithianon, dodemorph, dodine, econazole, etaconazole, edifenphos, epoxiconazole, ethaboxam, ethirimol, ethridiazole, famoxadone, fenamidone, fenarimol, fenbuconazole, fencaramid, fenfuram, fenhexamide, fenoxanil, fenpiclonil, fenpropidin, fenpropimorph, fentin acetate, fentin hydroxide, ferbam, ferfurazoate, ferimzone, fluazinam, fludioxonil, flumetover, fluopicolide, fluoxastrobin, fluquinconazole, fluquinconazole, flusilazole, flusulfamide, flutolanil, flutriafol, fluxapyroxad, folpet, fosetyl-aluminum, fthalide, fuberidazole, furalaxyl, furametpyr, hexaconazole, hymexazole, guazatine, imazalil, imibenconazole, iminoctadine, iodicarb, ipconazole, iprobenfos, iprodione, iprovalicarb, isoconazole, isoprothiolane, isotianil, kasugamycin, kresoxim-methyl, mancozeb, mandipropamid, maneb, mapanipyrin, mefenoxam, mepronil, metalaxyl, metconazole, methasulfocarb, metiram, metominostrobinifenominostrobin, mepanipyrim, metrafenone, miconazole, myclobutanil, neo-asozin (ferric methanearsonate), nuarimol, octhilinone, ofurace, orysastrobin, oxadixyl, oxolinic acid, oxpoconazole, oxycarboxin, paclobutrazol, penconazole, pencycuron, penflufen, penthiopyrad, perfurazoate, phosphonic acid, phthalide, picobenzamid, picoxystrobin, polyoxin, probenazole, prochloraz, procymidone, propamocarb, propamocarb-hydrochloride, propiconazole, propineb, proquinazid, prothioconazole, pyraclostrobin, pyrametostrobin, pyraoxystrobin, pryazophos, pyrifenox, pyrimethanil, pyrifenox, pyriofenone, pyrolnitrine, pyroquilon, quinconazole, quinoxyfen, quintozene, silthiofam, simeconazole, spiroxamine, streptomycin, sulfur, tebuconazole, tebufloquin, techrazene, tecloftalam, tecnazene, tetraconazole, thiabendazole, thifluzamide, thiophanate, thiophanate-methyl, thiram, tiadinil, tolclofos-methyl, tolyfluanid, triadimefon, triadimenol, triarimol, triazoxide, tridemorph, trimorphamide, tricyclazole, trifloxystrobin, triflumizole, triforine, triticonazole, uniconazole, validamycin, valifenalate, vinclozolin, zineb, ziram, and zoxamide; nematocides such as aldicarb, imicyafos, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad.

Of note are fungicides and compositions comprising fungicides such as 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, azoxystrobin, copper hydroxide, cymoxanil, cyproconazole, difenoconazole, famoxadone, fenoxanil, ferimzone, flusilazole, flutolanil, fthalide, furametpyr, hexaconazole, isoprothiolane, isotianil, kasugamycin, mancozeb, metominostrobin, orysastrobin, pencycuron, penthiopyrad, picoxystrobin, probenazole, propiconazole, proquinazid, pyroquilon, simeconazole, tiadinil, tricyclazole, trifloxystrobin and validamycin.

In certain instances, combinations of a compound of this invention with other biologically active (particularly invertebrate pest control) compounds or agents (i.e. active ingredients) can result in a greater-than-additive (i.e. synergistic) effect. Reducing the quantity of active ingredients released in the environment while ensuring effective pest control is always desirable. When synergism of invertebrate pest control active ingredients occurs at application rates giving agronomically satisfactory levels of invertebrate pest control, such combinations can be advantageous for reducing crop production cost and decreasing environmental load.

Compounds of this invention and compositions thereof can be applied to plants genetically transformed to express proteins toxic to invertebrate pests (such as Bacillus thuringiensis delta-endotoxins). Such an application may provide a broader spectrum of plant protection and be advantageous for resistance management. The effect of the exogenously applied invertebrate pest control compounds of this invention may be synergistic with the expressed toxin proteins.

General references for these agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2^{nd} Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1, an N-oxide, or salt thereof, is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components can expand the spectrum of invertebrate pests controlled beyond the spectrum controlled by the compound of Formula 1 alone.

Table A lists specific combinations of a compound of Formula 1 with other invertebrate pest control agents illustrative of the mixtures, compositions and methods of the present invention. The first column of Table A lists the specific invertebrate pest control agents (e.g., “Abamectin” in the first line). The second column of Table A lists the mode of action (if known) or chemical class of the invertebrate pest control agents. The third column of Table A lists embodiment(s) of ranges of weight ratios for rates at which a compound of Formula 1 can be applied relative to an invertebrate pest control agent (e.g., “50:1 to 1:50” of a compound of Formula 1 relative to abamectin by weight). Thus, for example, the first line of Table A specifically discloses the combination of a compound of Formula 1 with abamectin can be applied in a weight ratio between 50:1 to 1:50. The remaining lines of Table A are to be construed similarly. Of further note Table A lists specific combinations of a compound of Formula 1 with other invertebrate pest control agents illustrative of the mixtures, compositions and methods of the present invention and includes additional embodiments of weight ratio ranges for application rates.

TABLE A

| Invertebrate Pest | Mode of Action or | Typical |
|---|---|---|
| Control Agent | Chemical Class | Weight Ratio |
| Abamectin | macrocyclic lactones | 50:1 to 1:50 |
| Acetamiprid | neonicotinoids | 150:1 to 1:200 |
| Amitraz | octopamine receptor ligands | 200:1 to 1:100 |
| Avermectin | macrocyclic lactones | 50:1 to 1:50 |
| Azadirachtin | ecdysone agonists | 100:1 to 1:120 |
| Beta-cyfluthrin | sodium channel modulators | 150:1 to 1:200 |
| Bifenthrin | sodium channel modulators | 100:1 to 1:10 |
| Buprofezin | chitin synthesis inhibitors | 500:1 to 1:50 |
| Cartap | nereistoxin analogs | 100:1 to 1:200 |
| Chlorantraniliprole | ryanodine receptor ligands | 100:1 to 1:120 |
| Chlorfenapyr | mitochondrial electron transport | 300:1 to 1:200 |
| | inhibitors | |
| Chlorpyrifos | cholinesterase inhibitors | 500:1 to 1:200 |
| Clothianidin | neonicotinoids | 100:1 to 1:400 |
| Cyantraniliprole | ryanodine receptor ligands | 100:1 to 1:120 |
| Cyfluthrin | sodium channel modulators | 150:1 to 1:200 |
| Cyhalothrin | sodium channel modulators | 150:1 to 1:200 |
| Cypermethrin | sodium channel modulators | 150:1 to 1:200 |
| Cyromazine | chitin synthesis inhibitors | 400:1 to 1:50 |
| Deltamethrin | sodium channel modulators | 50:1 to 1:400 |
| Dieldrin | cyclodiene insecticides | 200:1 to 1:100 |
| Dinotefuran | neonicotinoids | 150:1 to 1:200 |
| Diofenolan | molting inhibitor | 150:1 to 1:200 |
| Emamectin | macrocyclic lactones | 50:1 to 1:10 |
| Endosulfan | cyclodiene insecticides | 200:1 to 1:100 |
| Esfenvalerate | sodium channel modulators | 100:1 to 1:400 |
| Ethiprole | GABA-regulated chloride | 200:1 to 1:100 |
| | channel blockers | |
| Fenothiocarb | | 150:1 to 1:200 |
| Fenoxycarb | juvenile hormone mimics | 500:1 to 1:100 |
| Fenvalerate | sodium channel modulators | 150:1 to 1:200 |
| Fipronil | GABA-regulated chloride | 150:1 to 1:100 |
| | channel blockers | |
| Flonicamid | | 200:1 to 1:100 |
| Flubendiamide | ryanodine receptor ligands | 100:1 to 1:120 |
| Flufenoxuron | chitin synthesis inhibitors | 200:1 to 1:100 |
| Hexaflumuron | chitin synthesis inhibitors | 300:1 to 1:50 |
| Hydramethylnon | mitochondrial electron transport | 150:1 to 1:250 |
| | inhibitors | |
| Imidacloprid | neonicotinoids | 1000:1 to 1:1000 |
| Indoxacarb | sodium channel modulators | 200:1 to 1:50 |
| Lambda-cyhalothrin | sodium channel modulators | 50:1 to 1:250 |
| Lufenuron | chitin synthesis inhibitors | 500:1 to 1:250 |
| Metaflumizone | | 200:1 to 1:200 |
| Methomyl | cholinesterase inhibitors | 500:1 to 1:100 |
| Mcthoprene | juvenile hormone mimics | 500:1 to 1:100 |
| Methoxyfenozide | ecdysone agonists | 50:1 to 1:50 |
| Nitenpyram | neonicotinoids | 150:1 to 1:200 |
| Nithiazine | neonicotinoids | 150:1 to 1:200 |
| Novaluron | chitin synthesis inhibitors | 500:1 to 1:150 |
| Oxamyl | cholinesterase inhibitors | 200:1 to 1:200 |
| Pymetrozine | | 200:1 to 1:100 |
| Pyrethrin | sodium channel modulators | 100:1 to 1:10 |
| Pyridaben | mitochondrial electron transport | 200:1 to 1:100 |
| | inhibitors | |
| Pyridalyl | | 200:1 to 1:100 |
| Pyriproxyfen | juvenile hormone mimics | 500:1 to 1:100 |
| Ryanodine | ryanodine receptor ligands | 100:1 to 1:120 |
| Spinetoram | macrocyclic lactones | 150:1 to 1:100 |
| Spinosad | macrocyclic lactones | 500:1 to 1:10 |
| Spirodiclofen | lipid biosynthesis inhibitors | 200:1 to 1:200 |
| Spiromesifen | lipid biosynthesis inhibitors | 200:1 to 1:200 |
| Tebufenozide | ecdysone agonists | 500:1 to 1:250 |
| Thiacloprid | neonicotinoids | 100:1 to 1:200 |
| Thiamethoxam | neonicotinoids | 1250:1 to 1:1000 |
| Thiodicarb | cholinesterase inhibitors | 500:1 to 1:400 |
| Thiosultap-sodium | | 150:1 to 1:100 |
| Tralomethrin | sodium channel modulators | 150:1 to 1:200 |
| Triazamate | cholinesterase inhibitors | 250:1 to 1:100 |
| Triflumuron | chitin synthesis inhibitors | 200:1 to 1:100 |
| Bacillus thuringiensis | biological agents | 50:1 to 1:10 |
| Bacillus thuringiensis | biological agents | 50:1 to 1:10 |
| delta-endotoxin | | |
| NPV (e.g, Gemstar) | biological agents | 50:1 to 1:10 |

Of note is the composition of the present invention wherein the at least one additional biologically active compound or agent is selected from the Invertebrate Pest Control Agents listed in Table A above.

The weight ratios of a compound, including a compound of Formula 1, an N-oxide, or salt thereof, to the additional invertebrate pest control agent typically are between 1000:1 and 1:1000, with one embodiment being between 500:1 and 1:500, another embodiment being between 250:1 and 1:200 and another embodiment being between 100:1 and 1:50.

Listed below in Tables B1 to B22 are embodiments of specific compositions comprising a compound of Formula 1 (compound numbers (Cmpd. No.) refer to compounds in Index Tables A-N) and an additional invertebrate pest control agent.

TABLE B1

| Mixture | Cmpd. | | Invertebrate Pest Control |
|---|---|---|---|
| No. | No. | and | Agent |
| B1-1 | 12 | and | Abamectin |
| B1-2 | 12 | and | Acetamiprid |
| B1-3 | 12 | and | Amitraz |
| B1-4 | 12 | and | Avermectin |
| B1-5 | 12 | and | Azadirachtin |
| B1-5a | 12 | and | Bensultap |
| B1-6 | 12 | and | Beta-cyfluthrin |
| B1-7 | 12 | and | Bifenthrin |
| B1-8 | 12 | and | Buprofezin |
| B1-9 | 12 | and | Cartap |
| B1-10 | 12 | and | Chlorantraniliprole |
| B1-11 | 12 | and | Chlorfenapyr |
| B1-12 | 12 | and | Chlorpyrifos |
| B1-13 | 12 | and | Clothianidin |
| B1-14 | 12 | and | Cyantraniliprole |
| B1-15 | 12 | and | Cyfluthrin |
| B1-16 | 12 | and | Cyhalothrin |
| B1-17 | 12 | and | Cypermethrin |
| B1-18 | 12 | and | Cyromazine |
| B1-19 | 12 | and | Deltamethrin |
| B1-20 | 12 | and | Dieldrin |
| B1-21 | 12 | and | Dinotefuran |
| B1-22 | 12 | and | Diofenolan |
| B1-23 | 12 | and | Emamectin |
| B1-24 | 12 | and | Endosulfan |
| B1-25 | 12 | and | Esfenvalerate |
| B1-26 | 12 | and | Ethiprole |
| B1-27 | 12 | and | Fenothiocarb |
| B1-28 | 12 | and | Fenoxycarb |
| B1-29 | 12 | and | Fenvalerate |
| B1-30 | 12 | and | Fipronil |
| B1-31 | 12 | and | Flonicamid |
| B1-32 | 12 | and | Flubendiamide |
| B1-33 | 12 | and | Flufenoxuron |
| B1-34 | 12 | and | Hexaflumuron |
| B1-35 | 12 | and | Hydramethylnon |
| B1-36 | 12 | and | Imidacloprid |
| B1-37 | 12 | and | Indoxacarb |
| B1-38 | 12 | and | Lambda-cyhalothrin |
| B1-39 | 12 | and | Lufenuron |
| B1-40 | 12 | and | Metaflumizone |
| B1-41 | 12 | and | Methomyl |
| B1-42 | 12 | and | Methoprene |
| B1-43 | 12 | and | Methoxyfenozide |
| B1-44 | 12 | and | Nitenpyram |
| B1-45 | 12 | and | Nithiazine |
| B1-46 | 12 | and | Novaluron |
| B1-47 | 12 | and | Oxamyl |
| B1-48 | 12 | and | Phosmet |
| B1-49 | 12 | and | Pymetrozine |
| B1-50 | 12 | and | Pyrethrin |
| B1-51 | 12 | and | Pyridaben |
| B1-52 | 12 | and | Pyridalyl |
| B1-53 | 12 | and | Pyriproxyfen |
| B1-54 | 12 | anti | Ryanodine |
| B1-55 | 12 | and | Spinetoram |
| B1-56 | 12 | and | Spinosad |
| B1-57 | 12 | and | Spirodiclofen |
| B1-58 | 12 | and | Spiromesifen |
| B1-59 | 12 | and | Spirotetramat |
| B1-60 | 12 | and | Tebufenozide |
| B1-61 | 12 | and | Thiacloprid |
| B1-62 | 12 | and | Thiamethoxam |
| B1-63 | 12 | and | Thiodicarb |
| B1-64 | 12 | and | Thiosultap-sodium |
| B1-65 | 12 | and | Tolfenpyrad |
| B1-66 | 12 | and | Tralomethrin |
| B1-67 | 12 | and | Triazamate |
| B1-68 | 12 | and | Triflumuron |
| B1-69 | 12 | and | Bacillus thuringiensis |
| B1-70 | 12 | and | Bacillus thuringiensis |
| | | | delta-endotoxin |
| B1-71 | 12 | and | NPV (e.g., Gemstar) |

### Table B2

Table B2 is identical to Table B1, except that each reference to compound 12 in the column headed “Cmpd. No.” is replaced by a reference to compound 14. For example, the first mixture in Table B2 is designated B2-1 and is a mixture of compound 14 and the additional invertebrate pest control agent abamectin.

### Table B3

Table B3 is identical to Table B1, except that each reference to compound 12 in the column headed “Cmpd. No.” is replaced by a reference to compound 20. For example, the first mixture in Table B3 is designated B3-1 and is a mixture of compound 20 and the additional invertebrate pest control agent abamectin.

### Table B4

Table B4 is identical to Table B1, except that each reference to compound 12 in the column headed “Cmpd. No.” is replaced by a reference to compound 22. For example, the first mixture in Table B4 is designated B4-1 and is a mixture of compound 22 and the additional invertebrate pest control agent abamectin.

### Table B5

Table B5 is identical to Table B1, except that each reference to compound 12 in the column headed “Cmpd. No.” is replaced by a reference to compound 23. For example, the first mixture in Table B5 is designated B5-1 and is a mixture of compound 23 and the additional invertebrate pest control agent abamectin.

### Table B6

Table B6 is identical to Table B1, except that each reference to compound 12 in the column headed “Cmpd. No.” is replaced by a reference to compound 24. For example, the first mixture in Table B6 is designated B6-1 and is a mixture of compound 24 and the additional invertebrate pest control agent abamectin.

### Table B7

Table B7 is identical to Table B1, except that each reference to compound 12 in the column headed “Cmpd. No.” is replaced by a reference to compound 36. For example, the first mixture in Table B7 is designated B7-1 and is a mixture of compound 36 and the additional invertebrate pest control agent abamectin.

### Table B8

Table B8 is identical to Table B1, except that each reference to compound 12 in the column headed “Cmpd. No.” is replaced by a reference to compound 37. For example, the first mixture in Table B8 is designated B8-1 and is a mixture of compound 37 and the additional invertebrate pest control agent abamectin.

### Table B9

Table B9 is identical to Table B1, except that each reference to compound 12 in the column headed “Cmpd. No.” is replaced by a reference to compound 44. For example, the first mixture in Table B9 is designated B9-1 and is a mixture of compound 44 and the additional invertebrate pest control agent abamectin.

### Table B10

Table B10 is identical to Table B1, except that each reference to compound 12 in the column headed “Cmpd. No.” is replaced by a reference to compound 57. For example, the first mixture in Table B10 is designated B10-1 and is a mixture of compound 57 and the additional invertebrate pest control agent abamectin.

### Table B11

Table B11 is identical to Table B1, except that each reference to compound 12 in the column headed “Cmpd. No.” is replaced by a reference to compound 65. For example, the first mixture in Table B11 is designated B11-1 and is a mixture of compound 65 and the additional invertebrate pest control agent abamectin.

### Table B12

Table B12 is identical to Table B1, except that each reference to compound 12 in the column headed “Cmpd. No.” is replaced by a reference to compound 86. For example, the first mixture in Table B12 is designated B12-1 and is a mixture of compound 86 and the additional invertebrate pest control agent abamectin.

### Table B13

Table B13 is identical to Table B1, except that each reference to compound 12 in the column headed “Cmpd. No.” is replaced by a reference to compound 90. For example, the first mixture in Table B13 is designated B13-1 and is a mixture of compound 90 and the additional invertebrate pest control agent abamectin.

### Table B14

Table B14 is identical to Table B1, except that each reference to compound 12 in the column headed “Cmpd. No.” is replaced by a reference to compound 100. For example, the first mixture in Table B14 is designated B14-1 and is a mixture of compound 100 and the additional invertebrate pest control agent abamectin.

### Table B15

Table B15 is identical to Table B1, except that each reference to compound 12 in the column headed “Cmpd. No.” is replaced by a reference to compound 112. For example, the first mixture in Table B15 is designated B15-1 and is a mixture of compound 112 and the additional invertebrate pest control agent abamectin.

### Table B16

Table B16 is identical to Table B1, except that each reference to compound 12 in the column headed “Cmpd. No.” is replaced by a reference to compound 113. For example, the first mixture in Table B16 is designated B16-1 and is a mixture of compound 113 and the additional invertebrate pest control agent abamectin.

### Table B17

Table B17 is identical to Table B1, except that each reference to compound 12 in the column headed “Cmpd. No.” is replaced by a reference to compound 119. For example, the first mixture in Table B17 is designated B17-1 and is a mixture of compound 119 and the additional invertebrate pest control agent abamectin.

### Table B18

Table B18 is identical to Table B1, except that each reference to compound 12 in the column headed “Cmpd. No.” is replaced by a reference to compound 136. For example, the first mixture in Table B18 is designated B18-1 and is a mixture of compound 136 and the additional invertebrate pest control agent abamectin.

### Table B19

Table B19 is identical to Table B1, except that each reference to compound 12 in the column headed “Cmpd. No.” is replaced by a reference to compound 137. For example, the first mixture in Table B19 is designated B19-1 and is a mixture of compound 137 and the additional invertebrate pest control agent abamectin.

### Table B20

Table B20 is identical to Table B1, except that each reference to compound 12 in the column headed “Cmpd. No.” is replaced by a reference to compound 138. For example, the first mixture in Table B20 is designated B20-1 and is a mixture of compound 138 and the additional invertebrate pest control agent abamectin.

### Table B21

Table B21 is identical to Table B1, except that each reference to compound 12 in the column headed “Cmpd. No.” is replaced by a reference to compound 141. For example, the first mixture in Table B21 is designated B21-1 and is a mixture of compound 141 and the additional invertebrate pest control agent abamectin.

### Table B22

Table B22 is identical to Table B1, except that each reference to compound 12 in the column headed “Cmpd. No.” is replaced by a reference to compound 152. For example, the first mixture in Table B22 is designated B22-1 and is a mixture of compound 152 and the additional invertebrate pest control agent abamectin.

The specific mixtures listed in Tables B1 to B22 typically combine a compound of Formula 1 with the other invertebrate pest agent in the ratios specified in Table A.

Listed below in Tables C1 to C22 are embodiments of specific compositions comprising a compound of Formula 1 (compound numbers (Cmpd. No.) refer to compounds in Index Tables A-N) and an additional fungicide.

TABLE C1

| Mixture | Cmpd. | | |
|---|---|---|---|
| No. | No. | and | Fungicide |
| C1-1 | 12 | and | Probenazole |
| C1-2 | 12 | and | Tiadinil |
| C1-3 | 12 | and | Isotianil |
| C1-4 | 12 | and | Pyroquilon |
| C1-5 | 12 | and | Metominostrobin |
| C1-6 | 12 | and | Flutolanil |
| C1-7 | 12 | and | Validamycin |
| C1-8 | 12 | and | Furametpyr |
| C1-9 | 12 | and | Pencycuron |
| C1-10 | 12 | and | Simeconazole |
| C1-11 | 12 | and | Orysastrobin |
| C1-12 | 12 | and | Trifloxystrobin |
| C1-13 | 12 | and | Isoprothiolane |
| C1-14 | 12 | and | Azoxystrobin |
| C1-15 | 12 | and | Tricyclazole |
| C1-16 | 12 | and | Hexaconazole |
| C1-17 | 12 | and | Difenoconazole |
| C1-18 | 12 | and | Cyproconazole |
| C1-19 | 12 | and | Propiconazole |
| C1-20 | 12 | and | Fenoxanil |
| C1-21 | 12 | and | Ferimzone |
| C1-22 | 12 | and | Fthalide |
| C1-23 | 12 | and | Kasugamycin |
| C1-24 | 12 | and | Picoxystrobin |
| C1-25 | 12 | and | Penthiopyrad |
| C1-26 | 12 | and | Famoxadone |
| C1-27 | 12 | and | Cymoxanil |
| C1-28 | 12 | and | Proquinazid |
| C1-29 | 12 | and | Flusilazole |
| C1-30 | 12 | and | Mancozeb |
| C1-31 | 12 | and | Copper hydroxide |
| C1-32 | 12 | and | (a) |

(a) 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone

### Table C2

Table C2 is identical to Table C1, except that each reference to compound 12 in the column headed “Cmpd. No.” is replaced by a reference to compound 14. For example, the first mixture in Table C2 is designated C2-1 and is a mixture of compound 14 and the additional fungicide probenazole.

### Table C3

Table C3 is identical to Table C1, except that each reference to compound 12 in the column headed “Cmpd. No.” is replaced by a reference to compound 20. For example, the first mixture in Table C3 is designated C3-1 and is a mixture of compound 20 and the additional fungicide probenazole.

### Table C4

Table C4 is identical to Table C1, except that each reference to compound 12 in the column headed “Cmpd. No.” is replaced by a reference to compound 22. For example, the first mixture in Table C4 is designated C4-1 and is a mixture of compound 22 and the additional fungicide probenazole.

### Table C5

Table C5 is identical to Table C1, except that each reference to compound 12 in the column headed “Cmpd. No.” is replaced by a reference to compound 23. For example, the first mixture in Table C5 is designated C5-1 and is a mixture of compound 23 and the additional fungicide probenazole.

### Table C6

Table C6 is identical to Table C1, except that each reference to compound 12 in the column headed “Cmpd. No.” is replaced by a reference to compound 24 For example, the first mixture in Table C6 is designated C6-1 and is a mixture of compound 24 and the additional fungicide probenazole.

### Table C7

Table C7 is identical to Table C1, except that each reference to compound 12 in the column headed “Cmpd. No.” is replaced by a reference to compound 36. For example, the first mixture in Table C7 is designated C7-1 and is a mixture of compound 36 and the additional fungicide probenazole.

### Table C8

Table C8 is identical to Table C1, except that each reference to compound 12 in the column headed “Cmpd. No.” is replaced by a reference to compound 37. For example, the first mixture in Table C8 is designated C8-1 and is a mixture of compound 37 and the additional fungicide probenazole.

### Table C9

Table C9 is identical to Table C1, except that each reference to compound 12 in the column headed “Cmpd. No.” is replaced by a reference to compound 44. For example, the first mixture in Table C9 is designated C9-1 and is a mixture of compound 44 and the additional fungicide probenazole.

### Table C10

Table C10 is identical to Table C1, except that each reference to compound 12 in the column headed “Cmpd. No.” is replaced by a reference to compound 57. For example, the first mixture in Table C10 is designated C10-1 and is a mixture of compound 57 and the additional fungicide probenazole.

### Table C11

Table C11 is identical to Table C1, except that each reference to compound 12 in the column headed “Cmpd. No.” is replaced by a reference to compound 65. For example, the first mixture in Table C11 is designated C11-1 and is a mixture of compound 65 and the additional fungicide probenazole.

### Table C12

Table C12 is identical to Table C1, except that each reference to compound 12 in the column headed “Cmpd. No.” is replaced by a reference to compound 86. For example, the first mixture in Table C12 is designated C12-1 and is a mixture of compound 86 and the additional fungicide probenazole.

### Table C13

Table C13 is identical to Table C1, except that each reference to compound 12 in the column headed “Cmpd. No.” is replaced by a reference to compound 90. For example, the first mixture in Table C13 is designated C13-1 and is a mixture of compound 90 and the additional fungicide probenazole.

### Table C14

Table C14 is identical to Table C1, except that each reference to compound 12 in the column headed “Cmpd. No.” is replaced by a reference to compound 100. For example, the first mixture in Table C14 is designated C14-1 and is a mixture of compound 100 and the additional fungicide probenazole.

### Table C15

Table C15 is identical to Table C1, except that each reference to compound 12 in the column headed “Cmpd. No.” is replaced by a reference to compound 112. For example, the first mixture in Table C15 is designated C15-1 and is a mixture of compound 112 and the additional fungicide probenazole.

### Table C16

Table C16 is identical to Table C1, except that each reference to compound 12 in the column headed “Cmpd. No.” is replaced by a reference to compound 113. For example, the first mixture in Table C16 is designated C16-1 and is a mixture of compound 113 and the additional fungicide probenazole.

### Table C17

Table C17 is identical to Table C1, except that each reference to compound 12 in the column headed “Cmpd. No.” is replaced by a reference to compound 119. For example, the first mixture in Table C17 is designated C17-1 and is a mixture of compound 119 and the additional fungicide probenazole.

### Table C18

Table C18 is identical to Table C1, except that each reference to compound 12 in the column headed “Cmpd. No.” is replaced by a reference to compound 136. For example, the first mixture in Table C18 is designated C18-1 and is a mixture of compound 136 and the additional fungicide probenazole.

### Table C19

Table C19 is identical to Table C1, except that each reference to compound 12 in the column headed “Cmpd. No.” is replaced by a reference to compound 137. For example, the first mixture in Table C19 is designated C19-1 and is a mixture of compound 137 and the additional fungicide probenazole.

### Table C20

Table C20 is identical to Table C1, except that each reference to compound 12 in the column headed “Cmpd. No.” is replaced by a reference to compound 138. For example, the first mixture in Table C20 is designated C20-1 and is a mixture of compound 138 and the additional fungicide probenazole.

### Table C21

Table C21 is identical to Table C1, except that each reference to compound 12 in the column headed “Cmpd. No.” is replaced by a reference to compound 141. For example, the first mixture in Table C21 is designated C21-1 and is a mixture of compound 141 and the additional fungicide probenazole.

### Table C22

Table C22 is identical to Table C1, except that each reference to compound 12 in the column headed “Cmpd. No.” is replaced by a reference to compound 152. For example, the first mixture in Table C22 is designated C22-1 and is a mixture of compound 152 and the additional fungicide probenazole.

Invertebrate pests are controlled in agronomic and nonagronomic applications by applying one or more compounds of this invention, typically in the form of a composition, in a biologically effective amount, to the environment of the pests, including the agronomic and/or nonagronomic locus of infestation, to the area to be protected, or directly on the pests to be controlled.

Thus the present invention comprises a method for controlling an invertebrate pest in agronomic and/or nonagronomic applications, comprising contacting the invertebrate pest or its environment with a biologically effective amount of one or more of the compounds of the invention, or with a composition comprising at least one such compound or a composition comprising at least one such compound and a biologically effective amount of at least one additional biologically active compound or agent. Examples of suitable compositions comprising a compound of the invention and a biologically effective amount of at least one additional biologically active compound or agent include granular compositions wherein the additional active compound is present on the same granule as the compound of the invention or on granules separate from those of the compound of the invention.

Embodiments of the method of this invention include contacting the environment. Of note is the method wherein the environment is a plant. Also of note is the method wherein the environment is an animal. Also of note is the method wherein the environment is a seed.

To achieve contact with a compound or composition of the invention to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a compound of the invention can be applied to the plant foliage or the soil. Compounds of this invention can also be effectively delivered through plant uptake by contacting the plant with a composition comprising a compound of this invention applied as a soil drench of a liquid formulation, a granular formulation to the soil, a nursery box treatment or a dip of transplants. Of note is a composition of the present invention in the form of a soil drench liquid formulation. Also of note is a method for controlling an invertebrate pest comprising contacting the invertebrate pest or its environment with a biologically effective amount of a compound of the present invention or with a composition comprising a biologically effective amount of a compound of the present invention. Of further note is this method wherein the environment is soil and the composition is applied to the soil as a soil drench formulation. Of further note is that compounds of this invention are also effective by localized application to the locus of infestation. Other methods of contact include application of a compound or a composition of the invention by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. The compounds of this invention can also be impregnated into materials for fabricating invertebrate control devices (e.g., insect netting).

Compounds of this invention are also useful in seed treatments for protecting seeds from invertebrate pests. In the context of the present disclosure and claims, treating a seed means contacting the seed with a biologically effective amount of a compound of this invention, which is typically formulated as a composition of the invention. This seed treatment protects the seed from invertebrate soil pests and generally can also protect roots and other plant parts in contact with the soil of the seedling developing from the germinating seed. The seed treatment may also provide protection of foliage by translocation of the compound of this invention or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this invention can also increase vigor of plants growing from the seed.

One method of seed treatment is by spraying or dusting the seed with a compound of the invention (i.e. as a formulated composition) before sowing the seeds. Compositions formulated for seed treatment generally comprise a film former or adhesive agent. Therefore typically a seed coating composition of the present invention comprises a biologically effective amount of a compound of Formula 1 and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

Compounds of Formula 1 and their compositions, both alone and in combination with other insecticides, nematicides, and fungicides, are particularly useful in seed treatment for crops including, but not limited to, maize or corn, soybeans, cotton, cereal (e.g., wheat, oats, barley, rye and rice), potatoes, vegetables and oilseed rape.

Other insecticides or nematicides with which compounds of Formula 1 can be formulated to provide mixtures useful in seed treatment include but are not limited to abamectin, acetamiprid, acrinathrin, amitraz, avermectin, azadirachtin, bensultap, bifenthrin, buprofezin, cadusafos, carbaryl, carbofuran, cartap, chlorantraniliprole, chlorfenapyr, chlorpyrifos, clothianidin, cyantraniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, flufenoxuron, fluvalinate, formetanate, fosthiazate, hexaflumuron, hydramethylnon, imidacloprid, indoxacarb, lufenuron, metaflumizone, methiocarb, methomyl, methoprene, methoxyfenozide, nitenpyram, nithiazine, novaluron, oxamyl, pymetrozine, pyrethrin, pyridaben, pyridalyl, pyriproxyfen, ryanodine, spinetoram, spinosad, spirodiclofen, spiromesifen, spirotetramat, sulfoxaflor, tebufenozide, tetramethrin, thiacloprid, thiamethoxam, thiodicarb, thiosultap-sodium, tralomethrin, triazamate, triflumuron, Bacillus thuringiensis delta-endotoxins, all strains of Bacillus thuringiensis and all strains of Nucleo polyhedrosis viruses.

Fungicides with which compounds of Formula 1 can be formulated to provide mixtures useful in seed treatment include but are not limited to amisulbrom, azoxystrobin, boscalid, carbendazim, carboxin, cymoxanil, cyproconazole, difenoconazole, dimethomorph, fluazinam, fludioxonil, fluquinconazole, fluopicolide, fluoxastrobin, flutriafol, fluxapyroxad, ipconazole, iprodione, metalaxyl, mefenoxam, metconazole, myclobutanil, paclobutrazole, penflufen, picoxystrobin, prothioconazole, pyraclostrobin, sedaxane, silthiofam, tebuconazole, thiabendazole, thiophanate-methyl, thiram, trifloxystrobin and triticonazole.

Compositions comprising compounds of Formula 1 useful for seed treatment can further comprise bacteria and fungi that have the ability to provide protection from the harmful effects of plant pathogenic fungi or bacteria and/or soil born animals such as nematodes. Bacteria exhibiting nematicidal properties may include but are not limited to Bacillus firmus, Bacillus cereus, Bacillius subtiliis and Pasteuria penetrans. A suitable Bacillus firmus strain is strain CNCM I-1582 (GB-126) which is commercially available as BioNem™. A suitable Bacillus cereus strain is strain NCMM I-1592. Both Bacillus strains are disclosed in U.S. Pat. No. 6,406,690. Other suitable bacteria exhibiting nematicidal activity are B. amyloliquefaciens IN937a and B. subtilis strain GB03. Bacteria exhibiting fungicidal properties may include but are not limited to B. pumilus strain GB34. Fungal species exhibiting nematicidal properties may include but are not limited to Myrothecium verrucaria, Paecilomyces lilacinus and Purpureocillium lilacinum.

Seed treatments can also include one or more nematicidal agents of natural origin such as the elicitor protein called harpin which is isolated from certain bacterial plant pathogens such as Erwinia amylovora. An example is the Harpin-N-Tek seed treatment technology available as N-Hibit™ Gold CST.

Seed treatments can also include one or more species of legume-root nodulating bacteria such as the microsymbiotic nitrogen-fixing bacteria Bradyrhizobium japonicum. These inocculants can optionally include one or more lipo-chitooligosaccharides (LCOs), which are nodulation (Nod) factors produced by rhizobia bacteria during the initiation of nodule formation on the roots of legumes. For example, the Optimize® brand seed treatment technology incorporates LCO Promoter Technology™ in combination with an inocculant.

Seed treatments can also include one or more isoflavones which can increase the level of root colonization by mycorrhizal fungi. Mycorrhizal fungi improve plant growth by enhancing the root uptake of nutrients such as water, sulfates, nitrates, phosphates and metals. Examples of isoflavones include, but are not limited to, genistein, biochanin A, formononetin, daidzein, glycitein, hesperetin, naringenin and pratensein. Formononetin is available as an active ingredient in mycorrhizal inocculant products such as PHC Colonize® AG.

Seed treatments can also include one or more plant activators that induce systemic acquired resistance in plants following contact by a pathogen. An example of a plant activator which induces such protective mechanisms is acibenzolar-S-methyl.

The treated seed typically comprises a compound of the present invention in an amount from about 0.1 g to 1 kg per 100 kg of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this invention can be incorporated into a bait composition that is consumed by an invertebrate pest or used within a device such as a trap, bait station, and the like. Such a bait composition can be in the form of granules which comprise (a) active ingredients, namely a biologically effective amount of a compound of Formula 1 an NV-oxide, or salt thereof; (b) one or more food materials; optionally (c) an attractant, and optionally (d) one or more humectants. Of note are granules or bait compositions which comprise between about 0.001-5% active ingredients, about 40-99% food material and/or attractant; and optionally about 0.05-10% humectants, which are effective in controlling soil invertebrate pests at very low application rates, particularly at doses of active ingredient that are lethal by ingestion rather than by direct contact. Some food materials can function both as a food source and an attractant. Food materials include carbohydrates, proteins and lipids. Examples of food materials are vegetable flour, sugar, starches, animal fat, vegetable oil, yeast extracts and milk solids. Examples of attractants are odorants and flavorants, such as fruit or plant extracts, perfume, or other animal or plant component, pheromones or other agents known to attract a target invertebrate pest. Examples of humectants, i.e. moisture retaining agents, are glycols and other polyols, glycerine and sorbitol. Of note is a bait composition (and a method utilizing such a bait composition) used to control at least one invertebrate pest selected from the group consisting of ants, termites and cockroaches. A device for controlling an invertebrate pest can comprise the present bait composition and a housing adapted to receive the bait composition, wherein the housing has at least one opening sized to permit the invertebrate pest to pass through the opening so the invertebrate pest can gain access to the bait composition from a location outside the housing, and wherein the housing is further adapted to be placed in or near a locus of potential or known activity for the invertebrate pest.

The compounds of this invention can be applied without other adjuvants, but most often application will be of a formulation comprising one or more active ingredients with suitable carriers, diluents, and surfactants and possibly in combination with a food depending on the contemplated end use. One method of application involves spraying a water dispersion or refined oil solution of a compound of the present invention. Combinations with spray oils, spray oil concentrations, spreader stickers, adjuvants, other solvents, and synergists such as piperonyl butoxide often enhance compound efficacy. For nonagronomic uses such sprays can be applied from spray containers such as a can, a bottle or other container, either by means of a pump or by releasing it from a pressurized container, e.g., a pressurized aerosol spray can. Such spray compositions can take various forms, for example, sprays, mists, foams, fumes or fog. Such spray compositions thus can further comprise propellants, foaming agents, etc. as needed for application. Of note is a spray composition comprising a biologically effective amount of a compound or a composition of the present invention and a carrier. One embodiment of such a spray composition comprises a biologically effective amount of a compound or a composition of the present invention and a propellant. Representative propellants include, but are not limited to, methane, ethane, propane, butane, isobutane, butene, pentane, isopentane, neopentane, pentene, hydrofluorocarbons, chlorofluorocarbons, dimethyl ether, and mixtures of the foregoing. Of note is a spray composition (and a method utilizing such a spray composition dispensed from a spray container) used to control at least one invertebrate pest selected from the group consisting of mosquitoes, black flies, stable flies, deer flies, horse flies, wasps, yellow jackets, hornets, ticks, spiders, ants, gnats, and the like, including individually or in combinations.

Nonagronomic uses refer to invertebrate pest control in the areas other than fields of crop plants. Nonagronomic uses of the present compounds and compositions include control of invertebrate pests in stored grains, beans and other foodstuffs, and in textiles such as clothing and carpets. Nonagronomic uses of the present compounds and compositions also include invertebrate pest control in ornamental plants, forests, in yards, along roadsides and railroad rights of way, and on turf such as lawns, golf courses and pastures. Nonagronomic uses of the present compounds and compositions also include invertebrate pest control in houses and other buildings which may be occupied by humans and/or companion, farm, ranch, zoo or other animals. Nonagronomic uses of the present compounds and compositions also include the control of pests such as termites that can damage wood or other structural materials used in buildings.

For agronomic applications, the rate of application required for effective control (i.e. “biologically effective amount”) will depend on such factors as the species of invertebrate to be controlled, the pest's life cycle, life stage, its size, location, time of year, host crop or animal, feeding behavior, mating behavior, ambient moisture, temperature, and the like. Under normal circumstances, application rates of about 0.01 to 2 kg of active ingredients per hectare are sufficient to control pests in agronomic ecosystems, but as little as 0.0001 kg/hectare may be sufficient or as much as 8 kg/hectare may be required. For nonagronomic applications, effective use rates will range from about 1.0 to 50 mg/square meter but as little as 0.1 mg/square meter may be sufficient or as much as 150 mg/square meter may be required. One skilled in the art can easily determine the biologically effective amount necessary for the desired level of invertebrate pest control.

Representative compounds of this invention prepared by the methods described herein are shown in Index Tables A-N. For mass spectral data (AP⁺ (M+1)), the numerical value reported is the molecular weight of the parent molecular ion (M) formed by addition of H⁺ (molecular weight of 1) to the molecule to give a M+1 peak observed by mass spectrometry using atmospheric pressure chemical ionization (AP⁺). The alternate molecular ion peaks (e.g., M+2 or M+4) that occur with compounds containing multiple halogens are not reported.

The following abbreviations are used in the Index Tables which follow: Cmpd means Compound, Me is methyl, Et is ethyl, Ph is phenyl, CHO is formyl and CN is cyano.

INDEX TABLE A

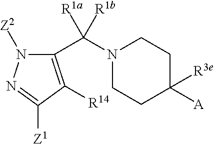

R^{1a} and R^{1b} are H, R¹⁴ is H

| | | | | | AP+ |
|---|---|---|---|---|---|
| Cmpd | Z¹ | Z² | A | R^{3e} | (M + 1) |
| 1 | 3-fluorophenyl | 3-chlorophenyl | CO₂Me | H | 462.4 |
| 2 | 3-(trifluoro- | 4-fluorophenyl | cyano | H | 429.5 |
| | methyl)phenyl | | | | |
| 3 | 4-chlorophenyl | 4-fluorophenyl | cyano | H | 396.4 |
| 4 | 4-methylphenyl | 4-fluorophenyl | CO₂Me | H | 408.5 |
| 5 | 4-methylphenyl | 4-fluorophenyl | cyano | H | 375.5 |
| 6 | 3-(trifluoro- | 4-fluorophenyl | CO₂Me | H | 462.4 |
| | methyl)phenyl | | | | |
| 7 | 4-chlorophenyl | 4-fluorophenyl | CO₂Me | H | 428.4 |
| 8 | 2,4-difluorophenyl | 4-fluorophenyl | CO₂Me | H | 430.5 |
| 9 | 2,4-difluorophenyl | 4-fluorophenyl | cyano | H | 397.4 |
| 10 | 4-chlorophenyl | 4-fluorophenyl | CO₂Et | H | 442.4 |
| 11 | 3-(trifluoro- | 4-fluorophenyl | CO₂Et | H | 476.4 |
| | methyl)phenyl | | | | |
| 12 | 3-(trifluoro- | 4-fluorophenyl | NHC(O)Me | H | 461.5 |
| | methyl)phenyl | | | | |
| 13 | 4-chlorophenyl | 4-fluorophenyl | NHC(O)Me | H | 427.5 |
| 14 | 3-(trifluoro- | 2-fluorophenyl | NHC(O)Me | H | 461.5 |
| | methyl)phenyl | | | | |
| 15 | 3-(trifluoro- | 2-fluorophenyl | cyano | H | 429.4 |
| | methyl)phenyl | | | | |
| 16 | 3-(trifluoro- | 2-fluorophenyl | CO₂Me | H | 462.5 |
| | methyl)phenyl | | | | |
| 17 | 3-(trifluoro- | 2-fluorophenyl | CO₂Et | H | 476.5 |
| | methyl)phenyl | | | | |
| 18 | 3-(trifluoro- | 4-chlorophenyl | NHC(O)Me | H | 477.5 |
| | methyl)phenyl | | | | |
| 19 | 3-(trifluoro- | 4-chlorophenyl | cyano | H | 445.4 |
| | methyl)phenyl | | | | |
| 20 | 3-(trifluoro- | 4-chlorophenyl | CO₂Me | H | 478.4 |
| | methyl)phenyl | | | | |
| 21 | 5-pyrimidinyl | 4-fluorophenyl | CO₂Me | H | 396.5 |
| 22 | 3-chloro-4- | 4-fluorophenyl | NHC(O)Me | H | 445.5 |
| | fluorophenyl | | | | |
| 23 | 3-chloro-4- | 4-fluorophenyl | cyano | H | 413.5 |
| | fluorophenyl | | | | |
| 24 | 3-chloro-4- | 4-fluorophenyl | CO₂Me | H | 446.4 |
| | fluorophenyl | | | | |
| 25 | 3-chloro-4- | 4-fluorophenyl | CO₂Et | H | 460.5 |
| | fluorophenyl | | | | |

INDEX TABLE B

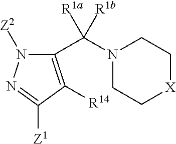

R^{1a} and R^{1b} are H, R¹⁴ is H

| | | | | AP+ |
|---|---|---|---|---|
| Cmpd | Z¹ | Z² | X | (M + 1) |
| 26 | 3-(trifluoromethyl)phenyl | 4-fluorophenyl | NCO₂Et | 477.4 |
| 27 | 3-(trifluoromethyl)phenyl | 4-fluorophenyl | NC(O)Me | 447.5 |
| 28 | 3-chloro-4-fluorophenyl | 4-fluorophenyl | NCO₂Et | 461.5 |
| 29 | 3-chloro-4-fluorophenyl | 4-fluorophenyl | O | 390.5 |
| 30 | 3-chloro-4-fluorophenyl | 4-fluorophenyl | NC(O)Me | 431.5 |

INDEX TABLE C

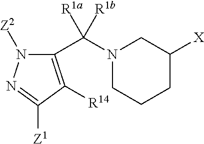

R^{1a} and R^{1b} are H, R¹⁴ is H

| | | | | AP+ |
|---|---|---|---|---|
| Cmpd | Z¹ | Z² | X | (M + 1) |
| 31 | 3-(trifluoromethyl)phenyl | 4-fluorophenyl | CO₂Me | 462.5 |
| 32 | 3-chloro-4-fluorophenyl | 4-fluorophenyl | CO₂Me | 446.6 |
| 35 | 4-chlorophenyl | 4-fluorophenyl | CO₂Et | 442.4 |

INDEX TABLE D

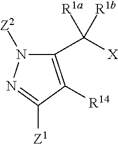

R^{1a} and R^{1b} are H, R¹⁴ is H

| | | | | AP+ |
|---|---|---|---|---|
| Cmpd | Z¹ | Z² | X | (M + 1) |
| 33 | 3-(trifluoromethyl)phenyl | 4-fluorophenyl | 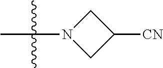 [SMILES: *N1CC(C#N)C1] | 401 |
| 34 | 3-(trifluoromethyl)phenyl | 4-fluorophenyl | 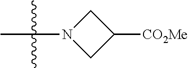 [SMILES: *N1CC(C(=O)OC)C1] | 434 |

INDEX TABLE E

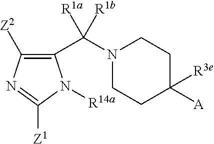

R^{1a} and R^{1b} are H

| | | | | | | m.p. | AP+ |
|---|---|---|---|---|---|---|---|
| Cmpd | Z¹ | Z² | R^{14a} | A | R^{3e} | (° C.) | (M + 1) |
| 36 | 3-(trifluoromethyl)phenyl | 2,4-difluorophenyl | H | cyano | H | | 447.1 |
| 37 | 3-(trifluoromethyl)phenyl | 2,4-difluorophenyl | H | CO₂Me | H | | 480.1 |
| 38 | 3-(trifluoromethyl)phenyl | 2,4-difluorophenyl | Me | cyano | H | | 461.1 |
| 39 | 3-(trifluoromethyl)phenyl | 2-fluorophenyl | H | cyano | H | | 429.0 |
| 40 | 3-(trifluoromethyl)phenyl | 2-fluorophenyl | Me | cyano | H | | 443.1 |
| 41 | 2-fluorophenyl | 4-chlorophenyl | H | cyano | H | | 395.4 |
| 73 | 4-chlorophenyl | 2,4-difluorophenyl | H | cyano | H | | 413.3 |
| 74 | 4-chlorophenyl | 2,4-difluorophenyl | H | NHC(O)Me | H | | 445.3 |
| 75 | 4-chlorophenyl | 2,4,6-trifluorophenyl | H | cyano | H | | 431.3 |
| 76 | 4-chlorophenyl | 2,4,6-trifluorophenyl | H | NHC(O)Me | H | | 463.3 |
| 77 | 2-fluorophenyl | 2,4-difluorophenyl | H | cyano | H | 90-92 | |
| 78 | 3-(trifluoromethyl)phenyl | 2,4,6-trifluorophenyl | H | cyano | H | | 465.3 |
| 79 | 4-chlorophenyl | 2-pyridinyl | H | cyano | H | | 378.3 |
| 80 | 2,3-difluorophenyl | 2,4-difluorophenyl | H | cyano | H | 114-116 | |
| 81 | 4-chlorophenyl | 2-pyridinyl | H | CO₂Me | H | | 411.3 |
| 82 | 3,4-difluorophenyl | 2,4-difluorophenyl | H | cyano | H | 107-109 | |
| 83 | 3,5-difluorophenyl | 2,4-difluorophenyl | H | cyano | H | 100-102 | |
| 84 | 3,4-dichlorophenyl | 2,4-difluorophenyl | H | cyano | H | 97-99 | |
| 85 | 3-chloro-4-fluorophenyl | 2,4-difluorophenyl | H | cyano | H | 194-196 | |
| 86 | 4-chloro-3-fluorophenyl | 2,4-difluorophenyl | H | cyano | H | 162-164 | |
| 87 | 3-nitrophenyl | 2,4-difluorophenyl | H | cyano | H | 177-179 | |
| 88 | 3-methoxyphenyl | 2,4-difluorophenyl | H | cyano | H | 165-167 | |
| 89 | 5-chlorothiophen-2-yl | 2,4-difluorophenyl | H | cyano | H | 146-148 | |
| 90 | 5-chloropyridin-2-yl | 2,4-difluorophenyl | H | cyano | H | 112-114 | |
| 91 | 3-(trifluoromethyl)phenyl | 2,4-difluorophenyl | H | cyano | H | 171-173 | |
| 92 | 2-fluorophenyl | 2,4-difluorophenyl | H | NHC(O)Me | H | 168-170 | |
| 93 | 3,4-dichlorophenyl | 2,4-difluorophenyl | H | NHC(O)Me | H | 219-221 | |
| 94 | 2,3-difluorophenyl | 2,4-difluorophenyl | H | 1,3,4-oxadiazol-2-yl | H | 178-180 | |
| 95 | 3,4-difluorophenyl | 2,4-difluorophenyl | H | cyano | H | 206-208 | |
| 96 | 4-chlorophenyl | 2,4-difluorophenyl | | NHC(O)Me | H | | 458 |

INDEX TABLE F

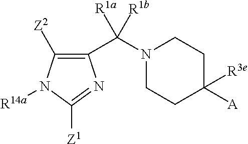

R^{1a} and R^{1b} are H

| | | | | | | AP+ |
|---|---|---|---|---|---|---|
| Cmpd | Z¹ | Z² | R^{14a} | A | R^{3e} | (M + 1) |
| 42 | 3-(trifluoro- | 2,4-difluorophenyl | Me | cyano | H | 461.1 |
| | methyl)phenyl | | | | | |
| 43 | 3-(trifluoro- | 2-fluorophenyl | Me | cyano | H | 443.1 |
| | methyl)phenyl | | | | | |

INDEX TABLE G

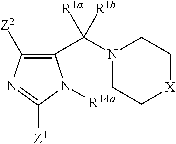

R^{1a} and R^{1b} are H

| | | | | | AP+ |
|---|---|---|---|---|---|
| Cmpd | Z¹ | Z² | R^{14a} | X | (M + 1) |
| 44 | 3-(trifluoromethyl)phenyl | 2,4-difluorophenyl | H | O | 424.1 |
| 45 | 3-(trifluoromethyl)phenyl | 2,4-difluorophenyl | Me | O | 438.1 |
| 46 | 3-chloro-4-fluorophenyl | 2-fluorophenyl | H | O | 406.1 |
| 47 | 3-chloro-4-fluorophenyl | 2-fluorophenyl | Me | O | 420.1 |
| 48 | 2-fluorophenyl | 4-chlorophenyl | H | O | 372.4 |
| 97 | 4-chlorophenyl | 2-pyridinyl | H | O | 355.3 |
| 98 | 4-chlorophenyl | 2,4,6-trifluorophenyl | H | O | 408.3 |
| 99 | 4-chlorophenyl | 2,4-difluorophenyl | H | O | 390.3 |

INDEX TABLE H

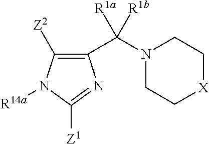

R^{1a} and R^{1b} are H

| | | | | | AP+ |
|---|---|---|---|---|---|
| Cmpd | Z¹ | Z² | R^{14a} | X | (M + 1) |
| 49 | 3-(trifluoromethyl)phenyl | 2,4-difluorophenyl | Me | O | 438.1 |
| 50 | 3-(trifluoromethyl)phenyl | 2-fluorophenyl | Me | O | 420.1 |

INDEX TABLE I

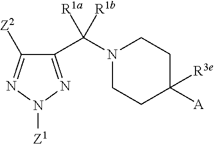

R^{1a} and R^{1b} are H

| | | | | | m.p. | AP+ |
|---|---|---|---|---|---|---|
| Cmpd | Z¹ | Z² | A | R^{3e} | (° C.) | (M + 1) |
| 51 | 3,4-dichlorophenyl | 2-fluorophenyl | cyano | H | 113-116 | |
| 52 | 3-(trifluoromethyl)phenyl | 2-fluorophenyl | cyano | H | | 430 |
| 53 | 4-chlorophenyl | 2-fluorophenyl | cyano | H | | 396 |
| 54 | 3-(trifluoromethyl)phenyl | 3-fluorophenyl | cyano | H | 106-108 | |
| 55 | 3,4-dichlorophenyl | 2-fluorophenyl | CO₂Me | H | | 463 |
| 56 | 3,4-dichlorophenyl | 2-fluorophenyl | NHC(O)Me | H | 169-171 | |
| 57 | 3-(trifluoromethyl)phenyl | 2-fluorophenyl | NHC(O)Me | H | 78-81 | |
| 58 | 3-(trifluoromethyl)phenyl | 2-fluorophenyl | CO₂Me | H | 83-86 | |
| 100 | 3-chloro-4-fluorophenyl | 2-fluorophenyl | NHC(O)Me | H | 152-154 | |
| 101 | 3-chloro-4-fluorophenyl | 2-fluorophenyl | CO₂Me | H | | 447 |
| 102 | 3-chloro-4-fluorophenyl | 2-fluorophenyl | cyano | H | | 414 |
| 103 | 4-(trifluoromethyl)phenyl | 2,4-difluorophenyl | cyano | H | | 448 |
| 104 | 4-(trifluoromethyl)phenyl | 2,4-difluorophenyl | NHC(O)Me | H | 191-193 | |
| 105 | 4-(trifluoromethyl)phenyl | 2,4-difluorophenyl | CO₂Me | H | | 481 |
| 106 | 4-chlorophenyl | 2,4-difluorophenyl | NHC(O)Me | H | 192-194 | |
| 107 | 4-chlorophenyl | 2,4-difluorophenyl | cyano | H | | 414 |
| 108 | 3-(trifluoromethyl)phenyl | 2,4-difluorophenyl | CO₂Me | H | 67-69 | |
| 109 | 3-(trifluoromethyl)phenyl | 2,4-difluorophenyl | NHC(O)Me | H | 68-73 | |
| 110 | 4-chlorophenyl | 2,4-difluorophenyl | CO₂Me | H | 192-194 | |
| 111 | 3-(trifluoromethyl)phenyl | 2,4-difluorophenyl | cyano | H | 94-96 | |
| 112 | 3-chloro-4-fluorophenyl | 2,4-difluorophenyl | NHC(O)Me | H | 155-157 | |
| 113 | 3-(trifluoromethyl)phenyl | 2,4,6-trifluorophenyl | NHC(O)Me | H | 147-149 | |
| 114 | 3-(trifluoromethyl)phenyl | 2,4,6-trifluorophenyl | CO₂Me | H | | 499 |
| 115 | 3-chloro-4-fluorophenyl | 2,4-difluorophenyl | CO₂Me | H | 88-90 | |
| 116 | 3-chloro-4-fluorophenyl | 2,4-difluorophenyl | cyano | H | | 432 |
| 117 | 3-(trifluoromethoxy)phenyl | 2,4-difluorophenyl | cyano | H | 101-103 | |
| 118 | 3-(trifluoromethoxy)phenyl | 2,4-difluorophenyl | NHC(O)Me | H | 142-144 | |
| 119 | 3-chloro-4-fluorophenyl | 2,4,6-trifluorophenyl | NHC(O)Me | H | 157-159 | |
| 120 | 3-chloro-4-fluorophenyl | 2,4,6-trifluorophenyl | cyano | H | | 450 |
| 121 | 6-(trifluoromethyl)pyridin-2-yl | 2,4-difluorophenyl | NHC(O)Me | H | | 481 |
| 122 | 6-(trifluoromethyl)pyridin-2-yl | 2,4-difluorophenyl | cyano | H | 126-128 | |
| 123 | 4-(trifluoromethyl)pyridin-2-yl | 2,4-difluorophenyl | cyano | H | 119-121 | |
| 124 | 4-(trifluoromethyl)pyridin-2-yl | 2,4-difluorophenyl | NHC(O)Me | H | 193-195 | |
| 125 | 5-(trifluoromethyl)pyridin-2-yl | 2,4-difluorophenyl | NHC(O)Me | H | 101-103 | |
| 126 | 5-(trifluoromethyl)pyridine2-yl | 2,4-difluorophenyl | cyano | H | 107-109 | |
| 127 | 3,4-dichlorophenyl | 4-fluorophenyl | cyano | H | 162-164 | |
| 128 | 3,4-dichlorophenyl | 4-fluorophenyl | NHC(O)Me | H | 186-188 | |
| 129 | 4-(trifluoromethyl)pyrimidin-2-yl | 2,4-difluorophenyl | NHC(O)Me | H | 97-99 | |
| 130 | 5-chloropyridin-2-yl | 2,4-difluorophenyl | NHC(O)Me | H | 129-133 | |
| 131 | 5-bromopyridin-2-yl | 2,4-difluorophenyl | cyano | H | 127-129 | |
| 132 | 5-bromopyridin-2-yl | 2,4-difluorophenyl | NHC(O)Me | H | | 491 |
| 133 | 2-(trifluoromethyl)pyrimidin-4-yl | 2,4-difluorophenyl | cyano | H | 158-160 | |
| 134 | 2-(trifluoromethyl)pyrimidin-4-yl | 2,4-difluorophenyl | NHC(O)Me | H | 174-175 | |

INDEX TABLE J

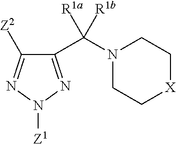

R^{1a} and R^{1b} are H

| | | | | m.p. | AP+ |
|---|---|---|---|---|---|
| Cmpd | Z¹ | Z² | X | (° C.) | (M + 1) |
| 59 | 3,4-dichlorophenyl | 2-fluorophenyl | O | | 407 |
| 60 | 3-(trifluoromethyl)phenyl | 2-fluorophenyl | O | 89-90 | |

INDEX TABLE K

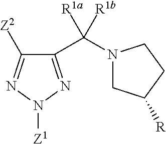

R^{1a} and R^{1b} are H

| | | | | m.p. | AP+ |
|---|---|---|---|---|---|
| Cmpd | Z¹ | Z² | Z² | (° C.) | (M + 1) |
| 135 | 3-(trifluoromethoxy)phenyl | 2,4-difluorophenyl | NHC(O)Me | 110-112 | |
| 136 | 3-chloro-4-fluorophenyl | 2,4-difluorophenyl | NHC(O)Me | 58-61 | |
| 137 | 3-(trifluoromethyl)phenyl | 2,4,6-trifluorophenyl | NHC(O)Me | 119-121 | |
| 138 | 3-chloro-4-fluorophenyl | 2,4,6-trifluorophenyl | NHC(O)Me | 134-137 | |
| 139 | 3-chloro-4-fluorophenyl | 4-fluorophenyl | NHC(O)Me | 174-177 | |
| 140 | 6-(trifluoromethyl)pyridin-2-yl | 2,4-difluorophenyl | NHC(O)Me | | 467 |
| 141 | 4-(trifluoromethyl)pyridin-2-yl | 2,4-difluorophenyl | NHC(O)Me | 144-146 | |
| 142 | 5-(trifluoromethyl)pyridin-2-yl | 2,4-difluorophenyl | NHC(O)Me | | 467 |
| 143 | 3,4-dichlorophenyl | 4-fluorophenyl | NHC(O)Me | 160-162 | |
| 144 | 3,4-dichlorophenyl | 4-fluorophenyl | NHC(O)CF₃ | 124-126 | |
| 145 | 3,4-dichlorophenyl | 4-fluorophenyl | NHC(O)cyclopropyl | 135-137 | |
| 146 | 3,4-dichlorophenyl | 4-fluorophenyl | NHC(O)OC(Me)₃ | 141-143 | |
| 147 | 3,4-dichlorophenyl | 4-fluorophenyl | NH₂ | 75-78 | |
| 148 | 5-chloropyridin-2-yl | 2,4-difluorophenyl | NHC(O)Me | 61-65 | |
| 149 | 5-bromopyridin-2-yl | 2,4-difluorophenyl | NHC(O)Me | | 477 |
| 150 | 2-(trifluoromethyl)pyridin-4-yl | 2,4-difluorophenyl | NHC(O)Me | 154-156 | |

INDEX TABLE L

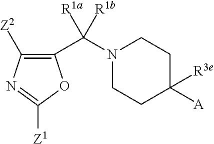

R^{1a} and R^{1b} are H

| | | | | | m.p. |
|---|---|---|---|---|---|
| Cmpd | Z¹ | Z² | A | R^{3e} | (° C.) |
| 61 | 4-chlorophenyl | 4-fluorophenyl | cyano | H | 119-121 |
| 62 | 4-chlorophenyl | 4-fluorophenyl | CO₂Me | H | 104-106 |
| 63 | 3,4-dichlorophenyl | 4-fluorophenyl | cyano | H | 166-168 |
| 64 | 3-(trifluoro- | 4-fluorophenyl | cyano | H | 143-145 |
| | methyl)phenyl | | | | |
| 65 | 3-(trifluoro- | 4-fluorophenyl | NHC(O)Me | H | 172-174 |
| | methyl)phenyl | | | | |
| 66 | 3,4-dichlorophenyl | 4-fluorophenyl | CO₂Me | H | 126-128 |
| 67 | 3,4-dichlorophenyl | 4-fluorophenyl | NHC(O)Me | H | 175-178 |
| 68 | 4-chlorophenyl | 4-fluorophenyl | NHC(O)Me | H | 181-183 |
| 69 | 3-(trifluoro- | 4-fluorophenyl | CO₂Me | H | 108-110 |
| | methyl)phenyl | | | | |

INDEX TABLE M

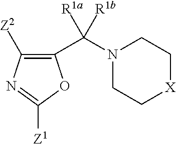

R^{1a} and R^{1b} are H

| | | | | m.p. |
|---|---|---|---|---|
| Cmpd | Z¹ | Z² | X | (° C.) |
| 70 | 3-(trifluoromethyl)phenyl | 4-fluorophenyl | O | 115-117 |
| 71 | 3,4-dichlorophenyl | 4-fluorophenyl | O | 121-123 |
| 72 | 4-chlorophenyl | 4-fluorophenyl | O | 111-113 |

INDEX TABLE N

| | | m.p. | AP+ |
|---|---|---|---|
| Cmpd | Structure | (° C.) | (M + 1) |
| 151 | 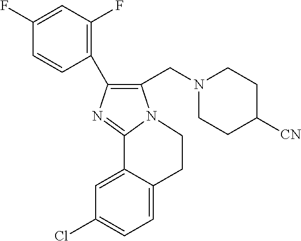 [SMILES: N#CC1CCN(Cc2c(-c3ccc(F)cc3F)nc3n2CCc2ccc(Cl)cc2-3)CC1] | | 439.4 |
| 152 | 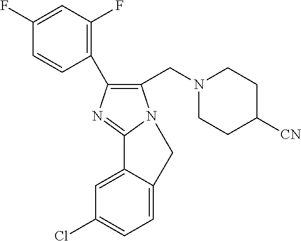 [SMILES: N#CC1CCN(Cc2c(-c3ccc(F)cc3F)nc3n2Cc2ccc(Cl)cc2-3)CC1] | | 425.3 |
| 153 | 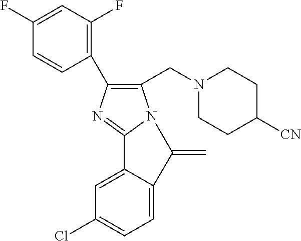 [SMILES: C=c1c2ccc(Cl)cc2c2nc(-c3ccc(F)cc3F)c(CN3CCC(C#N)CC3)n12] | | 437.4 |
| 154 | 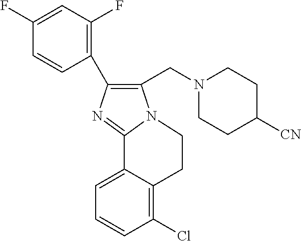 [SMILES: N#CC1CCN(Cc2c(-c3ccc(F)cc3F)nc3n2CCc2c(Cl)cccc2-3)CC1] | 193-195 | |
| 155 | 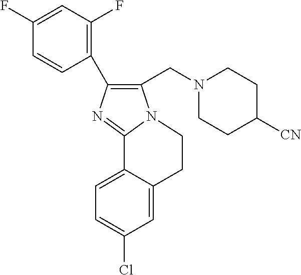 [SMILES: N#CC1CCN(Cc2c(-c3ccc(F)cc3F)nc3n2CCc2cc(Cl)ccc2-3)CC1] | 118-120 | |
| 156 | 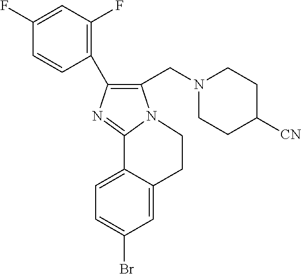 [SMILES: N#CC1CCN(Cc2c(-c3ccc(F)cc3F)nc3n2CCc2cc(Br)ccc2-3)CC1] | 130-134 | |
| 157 | 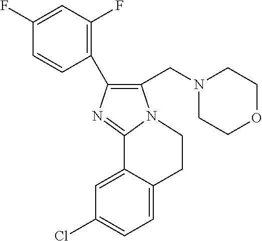 [SMILES: Fc1ccc(-c2nc3n(c2CN2CCOCC2)CCc2ccc(Cl)cc2-3)c(F)c1] | | 416.3 |
| 158 | 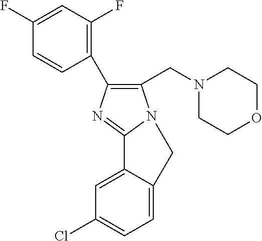 [SMILES: Fc1ccc(-c2nc3n(c2CN2CCOCC2)Cc2ccc(Cl)cc2-3)c(F)c1] | | 402.3 |
| 159 | 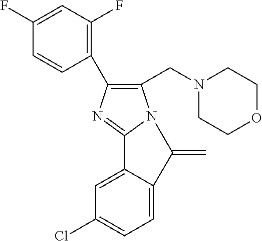 [SMILES: C=c1c2ccc(Cl)cc2c2nc(-c3ccc(F)cc3F)c(CN3CCOCC3)n12] | | 414.3 |
| 160 | 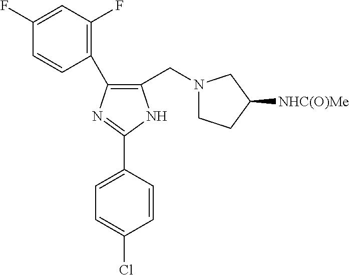 | | 431 |
| 161 | 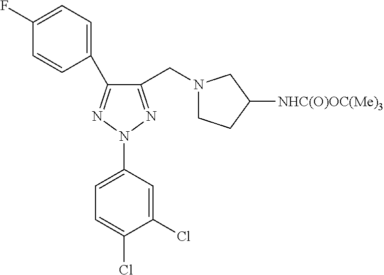 | 152-154 | |

The following Tests demonstrate the control efficacy of compounds of this invention on specific pests. “Control efficacy” represents inhibition of invertebrate pest development (including mortality) that causes significantly reduced feeding. The pest control protection afforded by the compounds is not limited, however, to these species. Compound numbers refer to compounds in Index Tables A-N.

### BIOLOGICAL EXAMPLES OF THE INVENTION

### Test A

For evaluating control of diamondback moth (Plutella xylostella) the test unit consisted of a small open container with a 12-14-day-old radish plant inside. This was pre-infested with ˜50 neonate larvae that were dispensed into the test unit via corn cob grits using a bazooka inoculator. The larvae moved onto the test plant after being dispensed into the test unit.

Test compounds were formulated using a solution containing 10% acetone, 90% water and 300 ppm X-77® Spreader Lo-Foam Formula non-ionic surfactant containing alkylarylpolyoxyethylene, free fatty acids, glycols and isopropanol (Loveland Industries, Inc. Greeley, Colo., USA). The formulated compounds were applied in 1 mL of liquid through a SUJ2 atomizer nozzle with ⅛ JJ custom body (Spraying Systems Co., Wheaton, Ill., USA) positioned 1.27 cm (0.5 inches) above the top of each test unit. Test compounds were sprayed at 250 ppm and/or 50 ppm, and replicated three times. After spraying of the formulated test compound, each test unit was allowed to dry for 1 h and then a black, screened cap was placed on top. The test units were held for 6 days in a growth chamber at 25° C. and 70% relative humidity. Plant feeding damage was then visually assessed based on foliage consumed.

Of the compounds of Formula 1 tested at 250 ppm, the following provided very good to excellent levels of control efficacy (40% or less feeding damage and/or 100% mortality): 2, 4, 6, 7, 8, 9, 35, 61 and 62.

Of the compounds of Formula 1 tested at 50 ppm, the following provided very good to excellent levels of control efficacy (40% or less feeding damage and/or 100% mortality): 2, 4, 6, 7, 8, 10, 11, 12, 13, 14, 15, 16, 17, 18, 20, 22, 23, 24, 25, 31, 36, 37, 38, 42, 44, 49, 51, 62, 75, 78, 80, 82, 83, 84, 85, 86, 87, 89, 90, 91, 98, 100, 101, 102, 103, 104, 105, 106, 107, 108, 109, 110, 111, 112, 113, 114, 115, 116, 118, 119, 120, 121, 135, 136, 137, 138, 139, 151, 152 and 155.

### Test B

For evaluating control of fall armyworm (Spodoptera frugiperda) the test unit consisted of a small open container with a 4-5-day-old maize (corn) plant inside. This was pre-infested (using a core sampler) with 10-15 1-day-old larvae on a piece of insect diet.

Test compounds were formulated and sprayed at 250 ppm and/or 50 ppm as described for Test A. The applications were replicated three times. After spraying, the test units were maintained in a growth chamber at 25° C. and 70% relative humidity and then visually rated as described for Test A.

Of the compounds of Formula 1 tested at 250 ppm, the following provided very good to excellent levels of control efficacy (40% or less feeding damage and/or 100% mortality): 32, 39, 55, 56, 57, 59, 61, 62, 63, 64, 65, 66, 67, 69, 71, 73, 77 and 99.

Of the compounds of Formula 1 tested at 50 ppm, the following provided very good to excellent levels of control efficacy (40% or less feeding damage and/or 100% mortality): 22, 23, 36, 39, 51, 53, 57, 61, 73, 77, 84, 85, 86, 90, 91, 100, 104, 106, 109, 110, 112, 113, 119, 135, 136, 137, 138, 151, 152 and 155.

### Test C

For evaluating control of green peach aphid (Myzus persicae) through contact and/or systemic means, the test unit consisted of a small open container with a 12-15-day-old radish plant inside. This was pre-infested by placing on a leaf of the test plant 30-40 aphids on a piece of leaf excised from a culture plant (cut-leaf method). The aphids moved onto the test plant as the leaf piece desiccated. After pre-infestation, the soil of the test unit was covered with a layer of sand.

Test compounds were formulated and sprayed at 250 ppm as described for Test A. The applications were replicated three times. After spraying of the formulated test compound, each test unit was allowed to dry for 1 h and then a black, screened cap was placed on top. The test units were held for 6 days in a growth chamber at 19-21° C. and 50-70% relative humidity. Each test unit was then visually assessed for insect mortality.

Of the compounds of Formula 1 tested at 250 ppm, the following resulted in at least 80% mortality: 40 and 114.

### Test D

For evaluating control of potato leafhopper (Empoasca fabae) through contact and/or systemic means, the test unit consisted of a small open container with a 5-6-day-old Soleil bean plant (primary leaves emerged) inside. White sand was added to the top of the soil and one of the primary leaves was excised prior to application.

Test compounds were formulated and sprayed at 250 ppm and/or 50 ppm, and the tests were replicated three times as described for Test A. After spraying, the test units were allowed to dry for 1 h before they were post-infested with 5 potato leafhoppers (18-21-day-old adults). A black, screened cap was placed on the top of each test unit, and the test units were held for 6 days in a growth chamber at 24° C. and 70% relative humidity. Each test unit was then visually assessed for insect mortality.

Of the compounds of Formula 1 tested at 250 ppm the following provided very good to excellent levels of control efficacy (80% or more mortality): 6, 12, 14, 16, 17, 20, 22, 24, 36, 37, 38, 44, 49, 55, 56, 57, 65, 66, 67, 68, 73, 74, 75, 76, 77, 78, 82, 83, 85, 89, 90, 91, 98, 100, 101, 103, 104, 105, 106, 108, 109, 112, 113, 114, 115, 118, 119, 121, 135, 136, 137, 138, 139, 151 and 155.

Of the compounds of Formula 1 tested at 50 ppm the following provided very good to excellent levels of control efficacy (80% or more mortality): 6, 12, 14, 22, 24, 36, 37, 44, 56, 57, 65, 67, 68, 73, 75, 82, 89, 98, 100, 104, 106, 108, 109, 112, 113, 114, 118, 119, 121, 124, 125, 132, 134, 136, 137, 138 and 141.

### Test E

For evaluating control of corn planthopper (Peregrinus maidis) through contact and/or systemic means, the test unit consisted of a small open container with a 3-4-day-old maize plant (spike) inside. White sand was added to the top of the soil prior to application. Test compounds were formulated and sprayed at 250 ppm, and replicated three times as described for Test A. After spraying, the test units were allowed to dry for 1 h before they were post-infested with ˜15-20 nymphs (18 to 21 day old) by sprinkling them onto the sand with a salt shaker. A black, screened cap was placed on the top of each test unit, and the test units were held for 6 days in a growth chamber at 22-24° C. and 50-70% relative humidity. Each test unit was then visually assessed for insect mortality.

Of the compounds of Formula 1 tested at 250 ppm the following provided very good to excellent levels of control efficacy (80% or more mortality): 92, 105 and 112.

What is claimed is:

1. A compound of Formula 1, an N-oxide, or salt thereof, 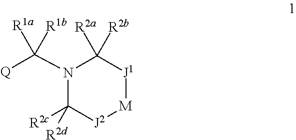
wherein
Q is 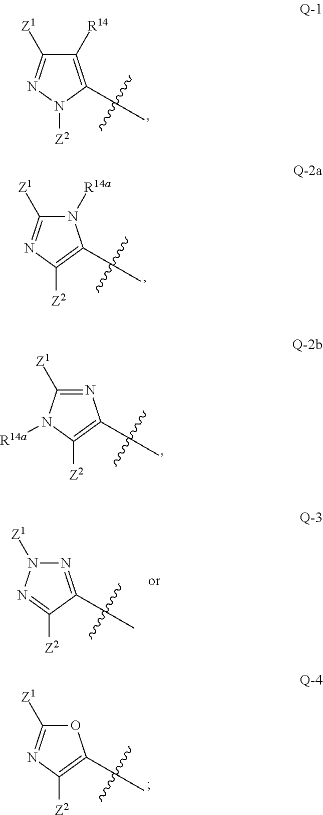
R^{1a} is H, cyano, C₁-C₆ alkyl, C₃-C₆ cycloalkyl, C₁-C₆ haloalkyl, C(O)OH, C(O)R^{5a}, C(O)OR^{6a} or C(O)NR^{7a}R^{8a};
R^{1b} is H or C₁-C₆ alkyl;
R^{2a} and R^{2c} are each independently H, halogen, cyano, C(X)R⁵, C(O)OR⁶, C(X)NR⁷R⁸, OR¹² or S(O)ₙR¹¹; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a};
R^{2b} and R^{2d} are each independently H, halogen, cyano, nitro, C(X)R⁵, C(O)OR⁶, C(X)NR⁷R⁸, NR⁹R¹⁰, OR¹², S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R⁴;
J¹ is a direct bond, —C(R^{3a}R^{3b})— or —C(R^{3a}R^{3b})C(R^{3a}R^{3b})—;
J² is a direct bond or —C(R^{3c}R^{3d})—;
M is —C(R^{3e})(A)-, —N(A¹)-, —O— or —S(O)ₙ—;
A is halogen, cyano, nitro, C(X)R⁵, C(O)OR⁶, C(X)NR⁷R⁸, C(X)NR^{7b}R⁸, NR⁹R¹⁰, S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, C₁-C₄ alkyl, C₁-C₄ haloalkyl, C(O)R^{5a}, C(O)OR^{6a}, C(O)NR^{7a}R^{8a}, NR^{9a}R^{10a}, OR^{12a} and S(O)ₙR^{11a}; or phenyl, a 5- or 6-membered heteroaromatic ring or a 7- to 11-membered heteroaromatic ring system, each ring or ring system unsubstituted or substituted with 1 to 3 R⁴;
A¹ is cyano, nitro, C(X)R⁵, C(O)OR⁶, C(X)NR⁷R⁸, C(X)NR^{7b}R⁸, NR⁹R¹⁰, S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, C₁-C₄ alkyl, C₁-C₄ haloalkyl, C(O)R^{5a}, C(O)OR^{6a}, C(O)NR^{7a}R^{8a}, NR^{9a}R^{10a}, OR^{12a} and S(O)ₙR^{11a}; or phenyl, a 5- or 6-membered heteroaromatic ring or a 7- to 11-membered heteroaromatic ring system, each ring or ring system unsubstituted or substituted with 1 to 3 R⁴; or benzyl unsubstituted or substituted with 1 to 3 R⁴;
each R^{3a} and R^{3c} are each independently H, halogen, cyano, C(X)R⁵, C(O)OR⁶, C(X)NR⁷R⁸, OR¹² or S(O)ₙR¹¹; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a};
each R^{3b} and R^{3d} are each independently H, halogen, cyano, nitro, C(X)R⁵, C(O)OR⁶, C(X)NR⁷R⁸, NR⁹R¹⁰, OR¹², S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R⁴;
R^{3e} is H, halogen, cyano, nitro, C(X)R⁵, C(O)OR⁶, C(X)NR⁷R⁸, NR⁹R¹⁰, OR¹², NR⁹R¹⁰, S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R⁴; or
R^{3e} and A can be taken together with the carbon atom to which they are attached to form a 3- to 7-membered ring containing ring members selected from carbon atoms and up to 2 heteroatoms independently selected from one oxygen atom, one sulfur atom, and up to 2 nitrogen atoms, wherein up to 2 carbon atom ring members are independently selected from C(═O) and C(═S) and the sulfur atom ring member is selected from S, S(O) or S(O)₂, said ring being unsubstituted or substituted with up to 4 substituents independently selected from the group consisting of halogen, cyano and C₁-C₄ alkyl; or
when any two substituents independently selected from the group consisting of R^{2a}, R^{2b}, R^{2c}, R^{2d}, R^{3a}, R^{3b}, R^{3c}, R^{3d} and R^{3e} are C₁-C₄ alkyl, then said two substituents can be taken together to form a ring;
Z¹ is phenyl substituted with 1 to 4 R^{4a}; or Z¹ is a 5- or 6-membered heteroaromatic ring or an 8- to 10-membered heteroaromatic bicyclic ring system, each ring unsubstituted or substituted with 1 to 3 R^{4a};
Z² is phenyl, unsubstituted or substituted with 1 to 4 R^{4b}; or Z² is a 5- or 6-membered heteroaromatic ring, each ring unsubstituted or substituted with 1 to 3 R^{4b};
each R⁴, R^{4a} and R^{4b} is independently halogen, cyano, nitro, C(X)R⁵, C(O)OR⁶, C(X)NR⁷R⁸, NR⁹R¹⁰, OR¹², S(O)ₙR¹¹ or SO₂NR⁹R¹⁰; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R⁵ is independently H; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{5a} is independently C₁-C₄ alkyl or C₁-C₄ haloalkyl;
each R⁶ is independently C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{6a} is independently C₁-C₄ alkyl or C₁-C₄ haloalkyl;
each R⁷ and R⁸ is independently H; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{7a} and R^{8a} is independently H, C₁-C₄ alkyl or C₁-C₄ haloalkyl;
each R^{7b} is independently N(R^{7a})₂, OH or OR^{12a};
each R⁹ and R¹⁰ is independently H, C(X)R⁵, C(O)OR⁶ or C(X)NR⁷R⁸; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{9a} and R^{10a} is independently H, C(X)R^{5a}, C(O)OR^{6a}, C(X)NR^{7a}R^{8a}, C₁-C₄ alkyl or C₁-C₄ haloalkyl;
each R¹¹ is independently C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{11a} is independently C₁-C₄ alkyl or C₁-C₄ haloalkyl;
each R¹² is independently H; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; or phenyl or a 5- or 6-membered heteroaromatic ring, each ring unsubstituted or substituted with 1 to 3 R¹³;
each R^{12a} is independently H, C₁-C₄ alkyl or C₁-C₄ haloalkyl;
each R¹³ is independently halogen, cyano, nitro, C(X)R^{5a}, C(O)OR^{6a}, C(X)NR^{7a}R^{8a}, NR^{9a}R¹⁰a, OR^{12a}, S(O)ₙR^{11a} or SO₂NR^{9a}R^{10a}; or C₁-C₆ alkyl, C₂-C₆ alkenyl, C₂-C₆ alkynyl, C₃-C₇ cycloalkyl or C₄-C₈ cycloalkylalkyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a};
R¹⁴ is H, halogen, cyano, nitro, C(X)R⁵, C(O)OR⁶, C(X)NR⁷R⁸, NR⁹R¹⁰, OR¹², S(O)ₙR¹¹ or SO₂NR⁹R¹⁰; or C₁-C₆ alkyl, C₂-C₆ alkenyl, C₂-C₆ alkynyl, C₃-C₇ cycloalkyl, C₄-C₈ cycloalkylalkyl or C₃-C₆ cycloalkenyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a};
R^{14a} is H; or C₁-C₆ alkyl, C₂-C₆ alkenyl, C₂-C₆ alkynyl or benzyl, each unsubstituted or substituted with at least one substituent independently selected from the group consisting of halogen, cyano, nitro, OR^{12a}, C₁-C₄ alkyl, C₁-C₄ haloalkyl and S(O)ₙR^{11a}; and when R^{14a} is C₁-C₆ alkyl or C₂-C₆ alkenyl, then R^{14a} can be bonded to Z¹ to form a ring;
each X is independently O or S; and
each n is independently 0, 1 or 2.

2. A compound of claim 1 wherein
R^{1a}, R^{1b}, R^{2a}, R^{2b}, R^{2c} and R^{2d} are H.

3. A compound of claim 2 wherein
J¹ is —C(R^{3a}R^{3b})—;
J² is a direct bond or —C(R^{3c}R^{3d})—; and
R^{3a}, R^{3b}, R^{3c} and R^{3d} are H.

4. A compound of claim 3 wherein
M is —C(R^{3e})(A)- or —O—; and
A is cyano, C(X)R⁵, C(O)OR⁶, C(X)NR⁷R⁸or NR⁹R¹⁰, S(O)ₙR¹¹ or SO₂NR⁹R¹⁰; or phenyl, or a 5- or 6-membered heteroaromatic ring, each ring unsubstituted or substituted with 1 to 3 R⁴.

5. A compound of claim 4 wherein
Q is Q-2a, Q-2b or Q-3.

6. A compound of claim 5 wherein
Z¹ is phenyl substituted with 1 to 4 R^{4a}; or pyridinyl, unsubstituted or substituted with 1 to 3 R^{4a}; and
Z² is phenyl, unsubstituted or substituted with 1 to 4 R^{4b}; or Z² is pyridinyl, unsubstituted or substituted with 1 to 3 R^{4b}.

7. A compound of claim 6 wherein
A is cyano, C(O)OR^{6a}, NR^{9a}C(O)R^{5a}, NHC(O)OR^{6a} or 1,3,4-oxadiazolyl; and
R^{9a} is H or C₁-C₄ alkyl.

8. A compound of claim 7 wherein
M is —C(R^{3e})(A)-;
R^{3e} is H; and
A is cyano or NHC(O)Me.

9. A composition comprising a compound of claim 1 and at least one additional component selected from the group consisting of surfactants, solid diluents and liquid diluents.

10. The composition of claim 9 further comprising at least one additional biologically active compound or agent.

11. The composition of claim 10 wherein the at least one additional biologically active compound or agent is selected from the group consisting of abamectin, acephate, acequinocyl, acetamiprid, acrinathrin, amidoflumet, amitraz, avermectin, azadirachtin, azinphos-methyl, benfuracarb, bensultap, bifenthrin, bifenazate, bistrifluoron, borate, buprofezin, cadusafos, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyantraniliprole, cycloprothrin, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenitrothion, fenothiocarb, fenoxycarb, fenpropathrin, fenvalerate, fipronil, flonicamid, flubendiamide, flucythrinate, flufenerim, flufenoxuron, fluvalinate, tau-fluvalinate, fonophos, formetanate, fosthiazate, halofenozide, hexaflumuron, hexythiazox, hydramethylnon, imidacloprid, indoxacarb, insecticidal soaps, isofenphos, lufenuron, malathion, meperfluthrin, metaflumizone, metaldehyde, methamidophos, methidathion, methiodicarb, methomyl, methoprene, methoxychlor, metofluthrin, monocrotophos, methoxyfenozide, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyrethrin, pyridaben, pyridalyl, pyrifluquinazon, pyriprole, pyriproxyfen, rotenone, ryanodine, silafluofen, spinetoram, spinosad, spirodiclofen, spiromesifen, spirotetramat, sulprofos, sulfoxaflor, tebufenozide, tebufenpyrad, teflubenzuron, tefluthrin, terbufos, tetrachlorvinphos, tetramethrin, tetramethylfluthrin, thiacloprid, thiamethoxam, thiodicarb, thiosultap-sodium, tolfenpyrad, tralomethrin, triazamate, trichlorfon, triflumuron, Bacillus thuringiensis delta-endotoxins, entomopathogenic bacteria, entomopathogenic viruses and entomopathogenic fungi

12. A method for controlling an invertebrate pest comprising contacting the invertebrate pest or its environment with a biologically effective amount of a compound of claim 1.

13. A treated seed comprising a compound of claim 1 in an amount of from about 0.0001 to 1% by weight of the seed before treatment.
